SEQUENCE LISTING (63 sequences)

SEQ ID NO: 1 - DNA, 630 bp, Reticulitermes flavipes
atgctggttgcaggactgcagttatttggaactgcattttgcgctctgcgactcttgtccacacgtaccgccccagtcatggcggcaactccagaagattggaaaaatgcatcatccatctatgatttcactgtaaaagatataaaggggcaagatgtgtctctggagaaatacagaggcgacgtggccattattgtgaatgtagcttcaaagtgtggcctgactcccacaaattacaaggaattggcagagcttcatgacaagtatgctgaatcgaaaggtctccgcatattagctttcccttgcaaccagtttaatagccaggaacctggaaacgcggaggaaatagtgtgctttgccaagtctaagaatgcaaattttgacatgtttgagaagattgatgtcaatggaaaccatgcacatccactgtggaagtacttgaagcacaagcaaggaggaactctaggagatttcatcaagtggaattttaccaagttcatcattgacaagaatggacagcctgtagagaggcatggtcccaacgttgatcccagtaaactggtgtccagcttggagaagtactggggtacccttgtgcccagaggcagccatcatcaccaccatcactaa

SEQ ID NO: 2 - protein, 209 aa, Reticulitermes flavipes
MLVAGLQLFGTAFCALRLLSTRTAPVMAATPEDWKNASSIYDFTVKDIKGQDVSLEKYRGDVAIIVNVASKCGLTPTNYKELAELHDKYAESKGLRILAFPCNQFNSQEPGNAEEIVCFAKSKNANFDMFEKIDVNGNHAHPLWKYLKHKQGGTLGDFIKWNFTKFIIDKNGQPVERHGPNVDPSKLVSSLEKYWGTLVPRGSHHHHHH

SEQ ID NO: 3 - DNA, 636 bp, Reticulitermes flavipes
atgaaggaaacacgggttctgatgatatttctgctcgtggctatgactgctgcccagtatccagtacgttattatgtacaacagccaggtgaccctgaacaccacgtcagctgccctaaatcagctgtgtgtaacttgattccttctaaggactctacagtctctggacaagtacagctgtaccaggcatctcaggctgaaccagttgaaataatagtttcggtacaaggtctgaaaccaccagggctacacggttttcatcttcatcgtgatggcaatactgatgatgactgcaaagcagctgggccacatttcaatccattcaatcacacacatggtgggccagaagatgaattccgacatgcaggagactttggaaacatattggcagatgaatatggtaatgcttcttaccgtataaagggaacacagatctcactatgtcctggcagtgtgggttactctgtgggccgtgcttttgtggtgcatgaaggcatagatgatcttggtaaaggtggtaatgaagagtctctcagaaccgggaatgctggaggtcgccttgcttgctgtgttgttgaagctgtcaattacggtacccttgtgcccagaggcagccatcatcaccaccatcactaa

SEQ ID NO: 4 - protein, 211 aa, Reticulitermes flavipes
MKETRVLMIFLLVAMTAAQYPVRYYVQQPGDPEHHVSCPKSAVCNLIPSKDSTVSGQVQLYQASQAEPVEIIVSVQGLKPPGLHGFHLHRDGNTDDDCKAAGPHFNPFNHTHGGPEDEFRHAGDFGNILADEYGNASYRIKGTQISLCPGSVGYSVGRAFVVHEGIDDLGKGGNEESLRTGNAGGRLACCVVEAVNYGTLVPRGSHHHHHH

SEQ ID NO: 5 - DNA, 28 bp, Unknown
  Laccase A (LacA) forward primer
tctagaatgttgccttgcgtcctgcttg

SEQ ID NO: 6 - DNA, 54 bp, Unknown
  Laccase A (LacA) reverse primer
cggccgttagtgatgatggtgatgatgacctccgttggtgttcacgggaggtgt

SEQ ID NO: 7 - DNA, 31 bp, Unknown
  AKR-forward primer
tctagaatgagtgcaaggttaacgaatagtg

SEQ ID NO: 8 - DNA, 27 bp, Unknown
  AKR truncated-forward primer
tctagaatggcgtttaagctagaaaaa

SEQ ID NO: 9 - DNA, 61 bp, Unknown
  AKR reverse primer
cggccgttagtgatgatggtgatgatgacctccgaattcaatgttaaatggatagtccttg

SEQ ID NO: 10 - DNA, 31 bp, Unknown
  GHF7-3 forward primer
gatcagatcttaatcaggatttcacctacac

SEQ ID NO: 11 - DNA, 30 bp, Unknown
  GHF7-3 reverse primer
gatcggtaccataagtgctatcaatcggac

SEQ ID NO: 12 - DNA, 27 bp, Unknown
  Primer sequence
gtcgacatgaggttacagacggtttgc

SEQ ID NO: 13 - DNA, 47 bp, Unknown
  Primer sequence
ctgcagttagtgatgatggtgatgatggtctaggaagcgttctggaa

SEQ ID NO: 14 - DNA, 89 bp, Unknown
  GHF9 cell-1 primer
ctagtctagactagatgaagatactccttgctattgcattaatgttgtcaacagtaatgtgggtgtcaacagctgcttacgactataag

SEQ ID NO: 15 - DNA, 56 bp, Unknown
  GHF9 cell-1 primer
tttccttttgcggccgcttagtgatgatggtgatgatgcacgccagccttgaggag

SEQ ID NO: 16 - DNA, 28 bp, Unknown
  primer sequence
tctagaatgttgccttgcgtcctgcttg

SEQ ID NO: 17 - DNA, 54 bp, Unknown; identical to SEQ ID NO: 6
  primer sequence

SEQ ID NO: 18 - protein, 8 aa, Reticulitermes flavipes
DAIDVGYR

SEQ ID NO: 19 - protein, 11 aa, Reticulitermes flavipes
DHKDYPFNIEF

SEQ ID NO: 20 - protein, 8 aa, Reticulitermes flavipes
DYPFNIEF

SEQ ID NO: 21 - protein, 8 aa, Reticulitermes flavipes
EDLFITSK

SEQ ID NO: 22 - protein, 12 aa, Reticulitermes flavipes
EGDDLFPEKDGK

SEQ ID NO: 23 - protein, 19 aa, Reticulitermes flavipes
HIDCAHVYGNEPEVGAAIK

SEQ ID NO: 24 - protein, 7 aa, Reticulitermes flavipes
KLIEFSK

SEQ ID NO: 25 - protein, 6 aa, Reticulitermes flavipes
LIEFSK

SEQ ID NO: 26 - protein, 8 aa, Reticulitermes flavipes
LVDQGLTK

SEQ ID NO: 27 - protein, 9 aa, Reticulitermes flavipes
REDLFITSK

SEQ ID NO: 28 - protein, 14 aa, Reticulitermes flavipes
SIGVSNFSSQQLER

SEQ ID NO: 29 - protein, 11 aa, Reticulitermes flavipes
SKPGEVTQAVK

SEQ ID NO: 30 - protein, 19 aa, Reticulitermes flavipes
SKPGEVTQAVKDAIDVGYR

SEQ ID NO: 31 - protein, 12 aa, Reticulitermes flavipes
TLKSLTSSCLQR

SEQ ID NO: 32 - protein, 17 aa, Reticulitermes flavipes
TLYSDVDYVDTWKELEK

SEQ ID NO: 33 - protein, 8 aa, Reticulitermes flavipes
TPAQILLR

SEQ ID NO: 34 - protein, 6 aa, Reticulitermes flavipes
VLANAR

SEQ ID NO: 35 - protein, 11 aa, Reticulitermes flavipes
YEKTPAQILLR

SEQ ID NO: 36 - protein, 12 aa, Reticulitermes flavipes
YQVQQGNITIPK

SEQ ID NO: 37 - DNA, 1738 bp, Reticulitermes flavipes
agccagtgaggtgttggaggtgaatgagtgcaaggttaacgaatagtgttagacgttttactgccacagttatggcgtttaagctagaaaaaactccgacsgtcaagttcaacaacggaattgaatttcccatctttggtctgggaacatggaagtccaaacctggtgaagtcactcaagctgtgaaggatgctattgacgttgggtaccgacacatcgattgcgctcacgtgtatggaaatgaacctgaagttggggccgcaattaaggccaagatcggcgagaaagtcgtgaagcgtgaggatctgtttatcacaagcaagctgtggaacacattccatcgaccagacttggttgcccctgctataaagcagactttgactgacttgggtttggattacttggacctgtatttgattcactggccaatggcatacaaggaaggtgatgacctctttccggagaaggatggtaaaactctgtacagtgatgtggactatgttgacacatggaaggagttggagaagttggtggatcagggcctcaccaagtcaattggggtgtcaaactttagttcacagcagctagaacgagttctggccaatgctagaatcaagccagttacgaaccaggttgagtgtcacccatatttgaaccaaaagaagttgatagagtttagtaaagcaaaaggtgtaacaatcactgcatacagcccgctgggctctccagatcgcccatgggccacgcctgatgatcctcaactgttggaagatccaaaagtgaaagctgtggctgcaaaatatgaaaagactcctgctcagatccttctgaggtaccaggtgcagcaaggtaatattacaatccccaaatctgtgacaaagtcacgtattgtagagaacgctcaaatctttgacttcgagctgtctgcagaggatgttgccacaattgattcttttgactgcaatggacgtgtctgtcacctggactggattaaagaccacaaggactatccatttaacattgaattctaagaagttgaagccacaaatgaagaatttgcaagaaaaatatgaagtcactgccagtccatggagcgagttacgtaacggggatggaggtgccttcacgatgactgcagtcagtacagtaatcaggaatacgcacttgtatgccagtaacgttgcagttttgatgctagtcgttcagcatccaagttggtatcatcatcttgatacatttttttcgtaattaggttaaattttaattacactggcttgtgtctgggcctgcttaattccaggcagccccagagttttggcattatatgcaacttacaaaaacaaatcatatgatgattagggtcattacttgcgtaaaaaatattacagttgcatattttccaattgctactactgcaataggacaggtttatgttgggacagaatttaaggttatgtaaaatacttcatgaattacagtgatgtatattcattttgtacatattttgccagctagtgttctttcagacactctgcccttcatttgttacaatatattcataagtatttctcccgtcattacaattgtttttctttgtaataatggtcgcatcagtgatctgatgagacatgttctagctaagctgtgtggcttcaaactagggcttcactgtacagaaaatactgaaataaagtgacttcatgaaaagtaaaaaaaaaaaaaaaaaaaaaaaaaaaaa

SEQ ID NO: 38 - DNA, 1052 bp, Reticulitermes flavipes
ggatcctatgagtgcaaggttaacgaattctgttagacgttttactgccacagttatggccttcaagctggaaaagacgcctaccgtcaagttcaacaacggtatcgagtttcctatctttggtctcggtacgtggaagtctaagcctggtgaggtcacccaagctgtcaaggacgctatcgacgtcggttaccgtcacattgactgtgctcatgtgtacggtaatgaacctgaagtcggcgcagctatcaaggctaagatcggtgaaaaggtggtgaagcgtgaggacctcttcatcacgtctaagctgtggaataccttccaccgtcctgatctggtcgctcctgctattaagcagacgctcaccgacctcggtctggattacctggacctgtacctgatccactggcctatggcttacaaggaaggtgacgacctgttccctgaaaaggatggtaagaccctgtattctgatgtcgactacgtcgacacttggaaggaactggagaagctggtggaccagggtctcaccaagtctatcggtgtgtctaacttctcttctcagcagctcgaacgtgtgctggctaacgcccgtatcaagcctgtcacgaaccaggtcgaatgccacccatatctgaaccaaaagaagctgatcgaattttccaaggctaagggtgtaacgatcaccgcttactctcctctgggctcccctgaccgtccatgggctacccctgatgaccctcaactgctcgaagaccctaaggtcaaggccgtggcagctaagtacgaaaagactcctgctcaaatcctgctgcgttaccaagtccagcaaggtaacatcactatccctaagtctgtgactaagtctcgtatcgtcgaaaacgctcagattttcgatttcgaactgtctgctgaagacgtggctaccatcgactctttcgactgcaatggtcgtgtctgccatctggactggatcaaggaccacaaggactatcctttcaacattgagttcggccgtggttctcatcaccaccatcatcatcattaattaa

SEQ ID NO: 39 - protein, 335 aa, Reticulitermes flavipes
MSARLTNSVRRFTATVMAFKLEKTPTVKFNNGIEFPIFGLGTWKSKPGEVTQAVKDAIDVGYRHIDCAHVYGNEPEVGAAIKAKIGEKVVKREDLFITSKLWNTFHRPDLVAPAIKQTLTDLGLDYLDLYLIHWPMAYKEGDDLFPEKDGKTLYSDVDYVDTWKELEKLVDQGLTKSIGVSNFSSQQLERVLANARIKPVTNQVECHPYLNQKKLIEFSKAKGVTITAYSPLGSPDRPWATPDDPQLLEDPKVKAVAAKYEKTPAQILLRYQVQQGNITIPKSVTKSRIVENAQIFDFELSAEDVATIDSFDCNGRVCHLDWIKDHKDYPFNIEF

SEQ ID NO: 40 - protein, 319 aa, Reticulitermes flavipes
MAFKLEKTPTVKFNNGIEFPIFGLGTWKSKPGEVTQAVKDAIDVGYRHIDCAHVYGNEPEVGAAIKAKIGEKVVKREDLFITSKLWNTFHRPDLVAPAIKQTLTDLGLDYLDLYLIHWPMAYKEGDDLFPEKDGKTLYSDVDYVDTWKELEKLVDQGLTKSIGVSNFSSQQLERVLANARIKPVTNQVECHPYLNQKKLIEFSKAKGVTITAYSPLGSPDRPWATPDDPQLLEDPKVKAVAAKYEKTPAQILLRYQVQQGNITIPKSVTKSRIVENAQIFDFELSAEDVATIDSFDCNGRVCHLDWIKDHKDYPFNIEF

SEQ ID NO: 41 - DNA, 693 bp, Reticulitermes flavipes
ctgcagatgaaattaatctctgttttgtttgcgcttgctgtagcgaaatcgttcgaagatcttgttaatactactgcgacgtcgaatgcatgtaccgtgacatcaaatcaacagggtacgtgtgatggcgttgcgtatgaattgtggatgtcaggatctggcggaagctgcacaatcaaaggtggtggtagtgctgcatttagcgcgaaatggagcaacagtggcgatttcttgtgtcgtgctggtcttggatcaggcagtgcaagtggcatcaaagctagctttgcatacacaaaatcaggcagtggtggtggctattcgtttattgggatctatggctggaccaccaaccctctggttgaatactacattgtggatgattggttctcaggaggtggtaactctggcggatctcagaaaggttctttcacccaagatggtgcgacatataacatctggcaacacactcagaacaaccagccatcgatccagggaacagcgacatttgagcaattcttcagcatccgctcaagccagcgcacatccggtgatatcaatatctcagctcactttgacaaatggagcagccttgggatgagaatgggcagcctgtatgaggcgaaattgctggttgaggctggtggtggtagtggaaacattgactattcgtctggcagcgtgaccaggggtacc

SEQ ID NO: 42 - protein, 229 aa, Reticulitermes flavipes
MKLISVLFALAVAKSFEDLVNTTATSNACTVTSNQQGTCDGVAYELWMSGSGGSCTIKGGGSAAFSAKWSNSGDFLCRAGLGSGSASGIKASFAYTKSGSGGGYSFIGIYGWTTNPLVEYYIVDDWFSGGGNSGGSQKGSFTQDGATYNIWQHTQNNQPSIQGTATFEQFFSIRSSQRTSGDINISAHFDKWSSLGMRMGSLYEAKLLVEAGGGSGNIDYSSGSVTRGT

SEQ ID NO: 43 - protein, 214 aa, Reticulitermes flavipes
FEDLVNTTATSNACTVTSNQQGTCDGVAYELWMSGSGGSCTIKGGGSAAFSAKWSNSGDFLCRAGLGSGSASGIKASFAYTKSGSGGGYSFIGIYGWTTNPLVEYYIVDDWFSGGGNSGGSQKGSFTQDGATYNIWQHTQNNQPSIQGTATFEQFFSIRSSQRTSGDINISAHFDKWSSLGMRMGSLYEAKLLVEAGGGSGNIDYSSGSVTRGT

SEQ ID NO: 44 - DNA, 1985 bp, Reticulitermes flavipes
atacggccctatcagtttacagatcggacgcctacattatgttgccttgcgtcctgcttgcttgcgcaattggtgtggcttctgcaacatcagtgctcctgaattcataccttcagcccaacgatgacattgatcgaaacacgtacctcctaaatgcaaaaagcaacaactgtgcccgtatatgcaatgggacagaggcgcccaaaatctgctactaccaatggacaattgagaactacgtgactctgtcagaagcgtgtgacaattgtcccttgaatgtgacggcctgttacaacgcacagtgcatcacagctgatggatatgagcgcagtatcctttcggtaaacaggaaactaccggggccttccatcgaggtgtgcctcagagacagagtaattgtggatataaccaacaacatggcagggaggactactagcatccactggcatggggtatttcagaaagggtcccagtacatggacggagttcccatggtaacccagtgcactatacatgagggtgacacattccggtacgactttatcgctaacaacgagggaactcatttctggcattcccatgacggtttgcagaagctcgatggcgtgacaggtaacttggtggttagggtgcctaaaaatttcgacccgaacggacaactgtacgatttcgatctaccagaacacaaaattttcatcagcgactggctacatctttccgcagatgaccactttcccggactccgagcgacaaatccaggacaagatgctaactcctttctcattaacggcagaggacgtaccttgattggaactcagtccaccaacacaccgtatgcgcagataaatgtgcagtggggcaggaggtaccggcttcgcattgtgggctccctgtgcactgtgtgccccacacagctcaccattgacgggcacaaaattacagtcatagccactgacggcaattctgtggctcctgccagagtcgactccctcatcatttactctggtgaaagatacgacgtcgtgttagaagccactaatacggaaggatcttactggatccatctaaaaggcctcgccacttgtgttggaagtagagtttaccagctgggggtgttgcaatatgaaaatacaacaaccaataaactgcatgctctgacacctgatccaggttacgacggattcccgcaaccagcaagctaccgggtcctgaacccagagaacgcaagctgtagcatcggctcgacaggcctatgcgtcacgcaactcgcgaactcggaccccgtgccacgggacatcctaacccagctcccggacatcaactatcttctccaatttggatttgaaactttcgactccagaagtttcttcaaagcttacgacagatattttgtcagcccctttctcgagttactcagcagtaccgtcaacaacatttctttcgtttcgcccccatctccgctcctctcacaaaggggggatgtaccagacgacatcctatgcccgacgggggctgatggcctgccccagtgtcccggaggaaactcctactgcacatgtgtccatgtcatcaaaatcaaactgggtgctttggtgcagatcatcctgtcggaccagtcacccaaatccgacctgaaccatccgttccatatacacggacatgcgttttacgtcctgggcatggggcaatacgctgcaggacagacggcgcaggacctccttaactccttgaagagtaacgtgagtagtgtgtcccctgcgccggttcttaaagataccgtcgcagttccatctggcggctacgcgatcatcaagttcagaccaaaaaaccctggttactggttccttcactgccacttcctgtaccatgtagcgaccgggatgagtgttgtgctccaggtgggagaaacaagtgactatccccctacaccagacggcttccccaagtgtggaagcttcacacctcccgtgaacaccaactgaagt

SEQ ID NO: 45 - DNA, 1489 bp, Reticulitermes flavipes
ccactaccagccgccatgaaggtcttcgtttgtcttctgtctgcactggcgctttgccaagctgcttacgactataagacagtactaagcaattcgctacttttctacgaggctcagcgatcgggaaaattgccgtctgatcagaaggtcacgtggaggaaggattccgcccttaacgacaagggccagaagggcgaggacctgacaggaggatactatgacgctggtgattttgtgaagttcggcttccctatggcgtacacagtcaccgtcctcgcttggggtgttatagactacgaatcagcgtattctgcagcaggagctctggatagtggtcgcaaggctcttaaatatggcacggactacttcctcaaggcgcacacggccgcgaacgaattctacggacaagtgggccagggagatgtcgaccacgcctactggggacgtccagaagacatgacgatgtccagacctgcctacaagatcgacacgtcgaaaccagggtctgacctggcagccgagacagccgccgccctcgctgcaactgccatcgcctacaagagtgctgacgcaacttattccaacaacttgatcacccacgccaagcagcttttcgacttcgccaacaattatcgcggcaaatacagtgattcaatcaccgacgcgaagaatttctacgcgtccggagactacaaggacgagttagtatgggcagccgcatggctctacagggcgaccaacgacaacacctatctgactaaagctgaatcgctatacaacgaattcggcctcggaaactggaacggtgccttcaactgggataacaagatctccggtgtacaggttctactggccaagctcacaagcaagcaggcatacaaggacaaggtacaaggctacgtcgattacttgatttcgtctcagaagaagacacccaagggtctcgtatacatcgaccagtggggtaccctgcgacatgctgccaattctgctctcattgctctgcaggcagccgacctgggtatcaatgctgctacttatcgcgcgtatgccaagaagcagatcgattacgcattgggtgatggaggtcgcagctacgtcgtaggatttggtactaacccacccgtacgccctcaccacagatccagctcgtgccctgacgcaccagccgtatgtgactggaacacgtacaacagcgccggccccaatgcccacgtactcaccggagccttggtgggtggtccagatagcaacgatagctacacggacgctcgcagcgattacatctccaacgaagtggccacagattacaacgctggcttccaatcagctgtcgctggtctcctcaaggctggcgtgtaaccgcacacagcactcaatgtctccctgtccactggacatgtgtacaatttgacaacgaaaatgtaatattcttcagaaaagtgcaataaaagttcacaattcaacacaaaaaaaaaaaaaaaaaaaa

SEQ ID NO: 46 - protein, 458 aa, Reticulitermes flavipes
MKILLAIALMLSTVMWVSTAAYDYKTVLSNSLLFYEAQRSGKLPSDQKVTWRKDSALNDKGQKGEDLTGGYYDAGDFVKFGFPMAYTVTVLAWGVIDYESAYSAAGALDSGRKALKYGTDYFLKAHTAANEFYGQVGQGDVDHAYWGRPEDMTMSRPAYKIDTSKPGSDLAAETAAALAATAIAYKSADATYSNNLITHAKQLFDFANNYRGKYSDSITDAKNFYASGDYKDELVWAAAWLYRATNDNTYLTKAESLYNEFGLGNWNGAFNWDNKISGVQVLLAKLTSKQAYKDKVQGYVDYLISSQKKTPKGLVYIDQWGTLRHAANSALIALQAADLGINAATYRAYAKKQIDYALGDGGRSYVVGFGTNPPVRPHHRSSSCPDAPAVCDWNTYNSAGPNAHVLTGALVGGPDSNDSYTDARSDYISNEVATDYNAGFQSAVAGLLKAGVHHHHHH

SEQ ID NO: 47 - protein, 438 aa, Reticulitermes flavipes
AYDYKTVLSNSLLFYEAQRSGKLPSDQKVTWRKDSALNDKGQKGEDLTGGYYDAGDFVKFGFPMAYTVTVLAWGVIDYESAYSAAGALDSGRKALKYGTDYFLKAHTAANEFYGQVGQGDVDHAYWGRPEDMTMSRPAYKIDTSKPGSDLAAETAAALAATAIAYKSADATYSNNLITHAKQLFDFANNYRGKYSDSITDAKNFYASGDYKDELVWAAAWLYRATNDNTYLTKAESLYNEFGLGNWNGAFNWDNKISGVQVLLAKLTSKQAYKDKVQGYVDYLISSQKKTPKGLVYIDQWGTLRHAANSALIALQAADLGINAATYRAYAKKQIDYALGDGGRSYVVGFGTNPPVRPHHRSSSCPDAPAVCDWNTYNSAGPNAHVLTGALVGGPDSNDSYTDARSDYISNEVATDYNAGFQSAVAGLLKAGVHHHHHH

SEQ ID NO: 48 - DNA, 1683 bp, Reticulitermes flavipes
gattctgacatcctcacgtctagggcgcttgacagagcaacgagatgaggttacagacggtttgcttcgtcatctttgtgacggcagtattcggggctgacgtcgataacgaaaccctcttcacgtttcctgaagactttaagttaggcgccgctacggcttcataccagattgaaggaggatggaatgcggatggaaagggtgtcaatatttgggacacactgacacatgagcgctcacaattagtggttgataaatcaagcggtgacgtggctgacgactcgtatcatctttataaggaggacgtgaagcttctgaagaacatgggggcacaactttatcgcttctctatatcttgggctcgcatcctgcctgaaggacatgataataaggtgaaccaggcgggcattgagtactacaacaagctcatagacgaacttctagacaatggaatagagccgatggttactatgtatcactgggatctaccccagacactccaagacctgggaggatggccaaatagagaattggcaaaatactccgagaattacgcccgcgttttatttcaaaactttggagaccgggttaaattgtggctcacattcaatgagcctctgactttcatggatgcatatgcatctgagacaggaatggctccatgaattgacacacccggtatcggcgattaccttgcggcacacactgtgatccttgcccatgccaatatctaccgtatgtatgagagggaattcaaagaggaacagaaaggaaaggttggtatcgcactcaacatacactggtgtgagccggtgactaattcgacaaaggacgttgaggcttgtgaaaggtatcaacagttcaacctgggaatatacgctcatcccatcttctctgtagagggcgattaccccagtgttttgaaagcgagggtagacgcaaacagcgtaacggaaggttacaccacatctcgtctacctaaattcactacagaggaagtagatttcatcagaggaacacatgatttcttgggtctgaatttctacactgctgtaacgggagcggatggagttgaaggggaacccccgtcgcggtacagagacatgggcgcgatcacatcacaggatccggactggcccgagtctgcttcttcatggctcagagttgtaccatggggattccgcaaggaacttaactggatcgcgaacgaatacggtaaccctcctatatacatcactgaaaatggcttctccgactacggtggcctcaatgatacagacagagtgctgtactacactgaacatttaaaggagatgctgaaggcaattcacatagatgaagttaacgtagtcggatacacagcctggagcctagtagacaatttcgaatggctgcgaggatatactgagaggttcggtatacatgaagtgaatttcaacgacccaagtcgcccacgagttcccaaggagtcagcaaaggtgctcacagagatcttcaacacaaggaggattccagaacgcttcctagactaacttcatattcaagacgcaaagacttatatcaaaaattaatttaaaagagggcttactgctgactgtaagttccctcaaaacagcaataaggtttatgatcatggaaaacacttcgaattaaataaacttatatacaaaaaaaaaaaaaaaa

SEQ ID NO: 49 - protein, 501 aa, Reticulitermes flavipes
MRLQTVCFVIFVTAVFGADVDNETLFTFPEDFKLGAATASYQIEGGWNADGKGVNIWDTLTHERSQLVVDKSSGDVADDSYHLYKEDVKLLKNMGAQLYRFSISWARILPEGHDNKVNQAGIEYYNKLIDELLDNGIEPMVTMYHWDLPQTLQDLGGWPNRELAKYSENYARVLFQNFGDRVKLWLTFNEPLTFMDAYASETGMAPSIDTPGIGDYLAAHTVILAHANIYRMYEREFKEEQKGKVGIALNIHWCEPVTNSTKDVEACERYQQFNLGIYAHPIFSVEGDYPSVLKARVDANSVTEGYTTSRLPKFTTEEVDFIRGTHDFLGLNFYTAVTGADGVEGEPPSRYRDMGAITSQDPDWPESASSWLRVVPWGFRKELNWIANEYGNPPIYITENGFSDYGGLNDTDRVLYYTEHLKEMLKAIHIDEVNVVGYTAWSLVDNFEWLRGYTERFGIHEVNFNDPSRPRVPKESAKVLTEIFNTRRIPERFLDHHHHHH

SEQ ID NO: 50 - protein, 484 aa, Reticulitermes flavipes
ADVDNETLFTFPEDFKLGAATASYQIEGGWNADGKGVNIWDTLTHERSQLVVDKSSGDVADDSYHLYKEDVKLLKNMGAQLYRFSISWARILPEGHDNKVNQAGIEYYNKLIDELLDNGIEPMVTMYHWDLPQTLQDLGGWPNRELAKYSENYARVLFQNFGDRVKLWLTFNEPLTFMDAYASETGMAPSIDTPGIGDYLAAHTVILAHANIYRMYEREFKEEQKGKVGIALNIHWCEPVTNSTKDVEACERYQQFNLGIYAHPIFSVEGDYPSVLKARVDANSVTEGYTTSRLPKFTTEEVDFIRGTHDFLGLNFYTAVTGADGVEGEPPSRYRDMGAITSQDPDWPESASSWLRVVPWGFRKELNWIANEYGNPPIYITENGFSDYGGLNDTDRVLYYTEHLKEMLKAIHIDEVNVVGYTAWSLVDNFEWLRGYTERFGIHEVNFNDPSRPRVPKESAKVLTEIFNTRRIPERFLDHHHHHH

SEQ ID NO: 51 - DNA, 1575 bp, Reticulitermes flavipes
gaattcatgggtcaccaccatcatcaccatcatcacggttcttctccggatccgatggcgtccgatcagctcgtgaactacaagaaaaagcagaccgataagactaaaattgtaaccggccatggtgccccggttgacaaccgtggcgcttctctcaccgtcggtcctcgtggtcctatgctgctccaagacattaccttcctcgatgaactggcgcactttgatcgcgagcgcattccagaacgtgttgttcatgcaaaaggtgcgggtgcgttcggttatttcgaggttactcatgatattacgaagtactgcaaagcatccgttttctctaagattggtaagaagactccaatcgcggttcgtttttctaccgtaggtggcgagtctggtagcgcggacaccgtccgtgacccgcgtggtttcgctgttaaattctacaccgaagacggtatctgggacctggtcggtaataacactccgatcttcttcatccgtgatccgctgctgttcccggttttcatccatacccagaaacgtaacccggcgacccacctgaaagactgcgacatgttctgggatttcctgtctctgcgcccagaatctacccaccaggttatgtttctcttcagcgatcgtggtatcccggacggcttccgtcacatgaatggttacggctctcatacgtttaaggcgattaatgataagaatgaggccgtgtatgtgaagttccactataaaaccaaccagggtatcaaaaacctcctcgcacagaaagcgtctgaagttgcggttgcagatccggactactctatccgcgacctctacaacgctattgcacgtggccagtacccatcttggaccctgtacatccaagttatgacgtttgaacaggcggaaaaattccgttggaacccgttcgacctgacgaaagtttggccgcatgccgaatatcctctgattccggtaggtaaactggttctcgatcgcaacccagcgaattactttgctgaggtagagcagattgcgttctctccggcgcacatggttccgggtatcgaaccgtctcctgataaaatgctgcaaggccgtctcttttcttactctgacacccaccgtcaccgcctcggtgcgaactatctccagatcccggtgaattgcccgtaccgtacccgtatcaccaactaccaacgtgacggccctcagaccttcacgaacaaccaagaaggcgctccgaactactacccgaactctttctctggtcctgaagatgttccgcactgcgctgcaattaagttcgcgtctacgggtgacgttgcgcgttacaactctggcgacgaagacaacttctcccagccatctctgttttggaaaaagaccctgaaaccggaagagcgtgaacgcctggtacaaaacatcgttgaccatgttaaagatgccgcggacttcgtccaggagcgtacggttaaaaacttttctcaggttgacgcggagtttggtcgtaagctgaccgaaggcctgcgtaaacactctaaaaacagctctatcgcatctgcgaacctcgagtaactgcag

SEQ ID NO: 52 - protein, 520 aa, Reticulitermes flavipes
MGHHHHHHHHGSSPDPMASDQLVNYKKKQTDKTKIVTGHGAPVDNRGASLTVGPRGPMLLQDITFLDELAHFDRERIPERVVHAKGAGAFGYFEVTHDITKYCKASVFSKIGKKTPIAVRFSTVGGESGSADTVRDPRGFAVKFYTEDGIWDLVGNNTPIFFIRDPLLFPVFIHTQKRNPATHLKDCDMFWDFLSLRPESTHQVMFLFSDRGIPDGFRHMNGYGSHTFKAINDKNEAVYVKFHYKTNQGIKNLLAQKASEVAVADPDYSIRDLYNAIARGQYPSWTLYIQVMTFEQAEKFRWNPFDLTKVWPHAEYPLIPVGKLVLDRNPANYFAEVEQIAFSPAHMVPGIEPSPDKMLQGRLFSYSDTHRHRLGANYLQIPVNCPYRTRITNYQRDGPQTFTNNQEGAPNYYPNSFSGPEDVPHCAAIKFASTGDVARYNSGDEDNFSQPSLFWKKTLKPEERERLVQNIVDHVKDAADFVQERTVKNFSQVDAEFGRKLTEGLRKHSKNSSIASANLE

SEQ ID NO: 53 - DNA, 1735 bp, Reticulitermes flavipes
ccagtgaggtgttggaggtgaatgagtgcaaggttaacgaatagtgttagacgttttactgccacagttatggcgtttaagctagaaaaaactccgacsgtcaagttcaacaacggaattgaatttcccatctttggtctgggaacatggaagtccaaacctggtgaagtcactcaagctgtgaaggatgctattgacgttgggtaccgacacatcgattgcgctcacgtgtatggaaatgaacctgaagttggggccgcaattaaggccaagatcggcgagaaagtcgtgaagcgtgaggatctgtttatcacaagcaagctgtggaacacattccatcgaccagacttggttgcccctgctataaagcagactttgactgacttgggtttggattacttggacctgtatttgattcactggccaatggcatacaaggaaggtgatgacctctttccggagaaggatggtaaaactctgtacagtgatgtggactatgttgacacatggaaggagttggagaagttggtggatcagggcctcaccaagtcaattggggtgtcaaactttagttcacagcagctagaacgagttctggccaatgctagaatcaagccagttacgaaccaggttgagtgtcacccatatttgaaccaaaagaagttgatagagtttagtaaagcaaaaggtgtaacaatcactgcatacagcccgctgggctctccagatcgcccatgggccacgcctgatgatcctcaactgttggaagatccaaaagtgaaagctgtggctgcaaaatatgaaaagactcctgctcagatccttctgaggtaccaggtgcagcaaggtaatattacaatccccaaatctgtgacaaagtcacgtattgtagagaacgctcaaatctttgacttcgagctgtctgcagaggatgttgccacaattgattcttttgactgcaatggacgtgtctgtcacctggactggattaaagaccacaaggactatccatttaacattgaattctaagaagttgaagccacaaatgaagaatttgcaagaaaaatatgaagtcactgccagtccatggagcgagttacgtaacggggatggaggtgccttcacgatgactgcagtcagtacagtaatcaggaatacgcacttgtatgccagtaacgttgcagttttgatgctagtcgttcagcatccaagttggtatcatcatcttgatacatttttttcgtaattaggttaaattttaattacactggcttgtgtctgggcctgcttaattccaggcagccccagagttttggcattatatgcaacttacaaaaacaaatcatatgatgattagggtcattamgcgtaaaaaatattacagttgcatattttccaattgctactactgcaataggacaggtttatgttgggacagaatttaaggttatgtaaaatacttcatgaattacagtgatgtatattcattttgtacatattttgccagctagtgttctttcagacactctgcccttcatttgttacaatatattcataagtatttctcccgtcattacaattgtttttctttgtaataatggtcgcatcagtgatctgatgagacatgttctagctaagctgtgtggcttcaaactagggcttcactgtacagaaaatactgaaataaagtgacttcatgaaaagtaaaaaaaaaaaaaaaaaaaaaaaaaaaaaa

SEQ ID NO: 54 - DNA, 1735 bp, Reticulitermes flavipes; identical to SEQ ID NO: 53

SEQ ID NO: 55 - protein, 335 aa, Reticulitermes flavipes; identical to SEQ ID NO: 39

SEQ ID NO: 56 - DNA, 1088 bp, Reticulitermes flavipes
atgtttgctttgattgtttttgccattcagctgctgcatgcacagggaaatcaggatttcacctacacgattaatggtactaaagttactgggcaaatagtgattgatcaagagtggagaggcaacaataccccaactgcaactgtgaatctttctagttttggtgtaactgtgaatggagataacgtgtcacagagattcaagacaggaactgctgtggggtcccgtatctatattcttgctccagggggaaaagcgtatgagaagttcaagttggtgaactctgagctgacgtttgatgttgatattagccagattccatgcggaatgaatgctgccatttacactgccgaattgcctgcagacggtgtaacacctggtcacgaagctggagcagcgtatggtggcggatactgtgatgcaaactatgttggaggagttggatgtgcagaatttgatattggtgaaagcaatgcacgtgcaacagtttatacaagtcatggatgcagcccgacgactggctttgcaaaacagggcagcattagctgtgacacaggtggaactggagccaacccgtaccgtgtggacaagaacttctatggcaatggttcatcattcactgtcaatactgcacagaaattcactgtggtgacgcaattcaaaggaaacccactgacttcgattgatcgtatctacatccaaggtaataaacaaacaaaacagccgaacaacattaataacaacttggatcgtatcagcccatcgcttgcggcaggacatgttctgatattctcgatctgggcttcggatggagatatgtcttggatggactgcaatgacaacggaccttgcaatgcaggccaggaaagttcacgttatttgggaacaaaactatccgatgctactgttacctacagcaatgttaggtggggtccgattgatagcacttattagataaagaagttgaagaccgagagggtcttttggtggaaaaaaaaattttttttgtttattgaagtgaagcaatcttattttttttgtagtaatttttttttgtggataaaataaaaattgagataaagatgcaa

SEQ ID NO: 57 - protein, 317 aa, Reticulitermes flavipes
MFALIVFAIQLLHAQGNQDFTYTINGTKVTGQIVIDQEWRGNNTPTATVNLSSFGVTVNGDNVSQRFKTGTAVGSRIYILAPGGKAYEKFKLVNSELTFDVDISQIPCGMNAAIYTAELPADGVTPGHEAGAAYGGGYCDANYVGGVGCAEFDIGESNARATVYTSHGCSPTTGFAKQGSISCDTGGTGANPYRVDKNFYGNGSSFTVNTAQKFTVVTQFKGNPLTSIDRIYIQGNKQTKQPNNINNNLDRISPSLAAGHVLIFSIWASDGDMSWMDCNDNGPCNAGQESSRYLGTKLSDATVTYSNVRWGPIDSTY

SEQ ID NO: 58 - protein, 337 aa, Reticulitermes flavipes
MVSAIVLYVLLAAAAHSAFADLNQDFTYTINGTKVTGQIVIDQEWRGNNTPTATVNLSSFGVTVNGDNVSQRFKTGTAVGSRIYILAPGGKAYEKFKLVNSELTFDVDISQIPCGMNAAIYTAELPADGVTPGHEAGAAYGGGYCDANYVGGVGCAEFDIGESNARATVYTSHGCSPTTGFAKQGSISCDTGGTGANPYRVDKNFYGNGSSFTVNTAQKFTVVTQFKGNPLTSIDRIYIQGNKQTKQPNNINNNLDRISPSLAAGHVLIFSIWASDGDMSWMDCNDNGPCNAGQESSRYLGTKLSDATVTYSNVRWGPIDSTYGTLVPRGSHHHHHH

SEQ ID NO: 59 - protein, 317 aa, Reticulitermes flavipes
DLNQDFTYTINGTKVTGQIVIDQEWRGNNTPTATVNLSSFGVTVNGDNVSQRFKTGTAVGSRIYILAPGGKAYEKFKLVNSELTFDVDISQIPCGMNAAIYTAELPADGVTPGHEAGAAYGGGYCDANYVGGVGCAEFDIGESNARATVYTSHGCSPTTGFAKQGSISCDTGGTGANPYRVDKNFYGNGSSFTVNTAQKFTVVTQFKGNPLTSIDRIYIQGNKQTKQPNNINNNLDRISPSLAAGHVLIFSIWASDGDMSWMDCNDNGPCNAGQESSRYLGTKLSDATVTYSNVRWGPIDSTYGTLVPRGSHHHHHH

SEQ ID NO: 60 - protein, 655 aa, Reticulitermes flavipes
MLPCVLLACAIGVASATSVLLNSYLQPNDDIDRNTYLLNAKSNNCARICNGTEAPKICYYQWTIENYVTLSEACDNCPLNVTACYNAQCITADGYERSILSVNRKLPGPSIEVCLRDRVIVDITNNMAGRTTSIHWHGVFQKGSQYMDGVPMVTQCTIHEGDTFRYDFIANNEGTHFWHSHDGLQKLDGVTGNLVVRVPKNFDPNGQLYDFDLPEHKIFISDWLHLSADDHFPGLRATNPGQDANSFLINGRGRTLIGTQSTNTPYAQINVQWGRRYRLRIVGSLCTVCPTQLTIDGHKITVIATDGNSVAPARVDSLIIYSGERYDVVLEATNTEGSYWIHLKGLATCVGSRVYQLGVLQYENTTTNKLHAITPDPGYDGFPQPASYRVLNPENASCSIGSTGLCVTQLANSDPVPRDILTQLPDINYLLQFGFETFDSRSFFKAYDRYFVSPFLELLSSTVNNISFVSPPSPLLSQRGDVPDDILCPTGADGLPQCPGGNSYCTCVHVIKIKLGALVQIILSDQSPKSDLNHPFHIHGHAFYVLGMGQYAAGQTAQDLLNSLKSNVSSVSPAPVLKDTVAVPSGGYAIIKFRPKNPGYWFLHCHFLYHVATGMSVVLQVGETSDYPPTPDGFPKCGSFTPPVNTNGGHHHHHH

SEQ ID NO: 61 - protein, 639 aa, Reticulitermes flavipes
TSVLLNSYLQPNDDIDRNTYLLNAKSNNCARICNGTEAPKICYYQWTIENYVTLSEACDNCPLNVTACYNAQCITADGYERSILSVNRKLPGPSIEVCLRDRVIVDITNNMAGRTTSIHWHGVFQKGSQYMDGVPMVTQCTIHEGDTFRYDFIANNEGTHFWHSHDGLQKLDGVTGNLVVRVPKNFDPNGQLYDFDLPEHKIFISDWLHLSADDHFPGLRATNPGQDANSFLINGRGRTLIGTQSTNTPYAQINVQWGRRYRLRIVGSLCTVCPTQLTIDGHKITVIATDGNSVAPARVDSLIIYSGERYDVVLEATNTEGSYWIHLKGLATCVGSRVYQLGVLQYENTTTNKLHAITPDPGYDGFPQPASYRVLNPENASCSIGSTGLCVTQLANSDPVPRDILTQLPDINYLLQFGFETFDSRSFFKAYDRYFVSPFLELLSSTVNNISFVSPPSPLLSQRGDVPDDILCPTGADGLPQCPGGNSYCTCVHVIKIKLGALVQIILSDQSPKSDLNHPFHIHGHAFYVLGMGQYAAGQTAQDLLNSLKSNVSSVSPAPVLKDTVAVPSGGYAIIKFRPKNPGYWFLHCHFLYHVATGMSVVLQVGETSDYPPTPDGFPKCGSFTPPVNTNGGHHHHHH

SEQ ID NO: 62 - protein, 639 aa, Reticulitermes flavipes
TSVLLNSYLQPNDDIDRNTYLLNAKSNNCARICNGTEAPKICYYQWTIENYVTLSEACDNCPLNVTACYNAQCITADGYERSILSVNRKLPGPSIEVCLRDRVIVDITNNMAGRTTSIHWHGVFQKGSQYMDGVPMVTQCTIHEGDTLRYDFIANNEGTHFWHSHDGLQKLDGVTGNLVVRVPKNFDPNGQLYDFDLPEHKIFISDWLHLSADDHFPGLRATNPGQDANSFLINGRGRTLIGTQSTNTPYAQINVQWGRRYRLRIVGSLCTVCPTQLTIDGHKITVIATDGNSVAPARVDSLIIYSGERYDVVLEATNTEGSYWIHLKGLVTCVGSRVYQLGVLQYENTTTNKLHALTPDPGYDGFPQPASYRVLNPENASCSIGSTGLCVTQLANSDPVPRDILTQLPDINYLLQFGFKIFDSRSFFKAYDRYFVSPFLDLVSSTVNNISSVSPPSPLLSQRGDVPDDVLCPTGADGLPQCPGGNSYCTCVHVIKIKLGALVQIILSDQTPKSGLNHPFHLHGHAFYVLGMGQYAAGQTAQDLLNSLKSNVSSVSPAPVLKDTIAVPSGGYAIIKFRPKNPGYWFLHCHFLYHVATGMSVVLQVGETSDYPPTPDGFPKCGSFTPPVNTNGGHHHHHH

SEQ ID NO: 63 - protein, 655 aa, Reticulitermes flavipes
MLPCVLLACAIGVASATSVLLNSYLQPNDDIDRNTYLLNAKSNNCARICNGTEAPKICYYQWTIENYVTLSEACDNCPLNVTACYNAQCITADGYERSILSVNRKLPGPSIEVCLRDRVIVDITNNMAGRTTSIHWHGVFQKGSQYMDGVPMVTQCTIHEGDTLRYDFIANNEGTHFWHSHDGLQKLDGVTGNLVVRVPKNFDPNGQLYDFDLPEHKIFISDWLHLSADDHFPGLRATNPGQDANSFLINGRGRTLIGTQSTNTPYAQINVQWGRRYRLRIVGSLCTVCPTQLTIDGHKITVIATDGNSVAPARVDSLIIYSGERYDVVLEATNTEGSYWIHLKGLVTCVGSRVYQLGVLQYENTTTNKLHALTPDPGYDGFPQPASYRVLNPENASCSIGSTGLCVTQLANSDPVPRDILTQLPDINYLLQFGFKIFDSRSFFKAYDRYFVSPFLDLVSSTVNNISSVSPPSPLLSQRGDVPDDVLCPTGADGLPQCPGGNSYCTCVHVIKIKLGALVQIILSDQTPKSGLNHPFHLHGHAFYVLGMGQYAAGQTAQDLLNSLKSNVSSVSPAPVLKDTIAVPSGGYAIIKFRPKNPGYWFLHCHFLYHVATGMSVVLQVGETSDYPPTPDGFPKCGSFTPPVNTNGGHHHHHH

(19) United States
(12) Patent Application Publication
(10) Pub. No.: US 2016/0369285 A1
(43) Pub. Date: Dec. 22, 2016

(54) TERMITE SUPEROXIDE DISMUTASES AND GLUTATHIONE PEROXIDASES FOR BIOMASS CONVERSION

(71) Applicants: Michael E. Scharg, Battle Ground, IN (US); Amit Sethi, Johnston, IA (US)

(72) Inventors: Michael E. Scharf, Battle Ground, IN (US); Amit Sethi, Johnston, IA (US)

(73) Assignee: Purdue Research Foundation, West Lafayette, IN (US)

(21) Appl. No.: 15/036,055

(22) PCT Filed: Nov. 12, 2014
(86) PCT No.: PCT/US14/00209

§ 371 Date: May 11, 2016

Related U.S. Application Data
(60) Provisional application No. 61/902,472, filed on Nov. 11, 2013.

(51) Int. Cl. C12N 15/52; C12N 15/86; C12N 9/42; C12N 7/00; C12N 9/02; C12N 9/08

(52) U.S. Cl. CPC C12N 15/52; C12N 9/0089; C12N 9/0065; C12N 9/0061; C12N 9/2437; C12Y 110/03002; C12Y 111/01009; C12Y 115/01001; C12Y 302/01004; C12N 7/00; C12N 15/86; C12N 2710/14043

(57) ABSTRACT

The present disclosure is generally related to enzymes, and to recombinant nucleic acid molecules encoding and/or expressing said enzymes, of the gut of the termite Reticulitermes flavipes. The disclosure further relates to a system combining said enzymes for substantially converting a plant lignocellulose to a fermentable sugar-based product.

This application claims priority to U.S. Provisional Patent Application 61/902,472 filed on Nov. 11, 2013, which is incorporated herein in its entirety.

This invention was made with government support under Grant No. DE-85538 S08-II awarded by the Department of Energy of the United States government. The government has certain rights in the invention.

### TECHNICAL FIELD

The present disclosure is generally related to enzymes, and to recombinant nucleic acid molecules encoding and/or expressing said enzymes, of the gut of the termite Reticulitermes flavipes. The disclosure further relates to a system combining said enzymes for substantially converting a plant lignocellulose to a fermentable sugar-based product.

### BACKGROUND

Lignocellulose is a sustainable global resource with a great deal of relevance to renewable energy production. In plants, lignocellulose provides key structural support for cell walls. Because it is plant-derived, lignocellulose is the most abundant and widespread bioenergy feedstock available on Earth. However, a major limitation in plant biomass utilization as a renewable energy source is the inefficiency of industrial lignocellulose depolymerization. This inefficiency increases energy inputs, reduces product yields, drives production costs higher, encourages political skepticism, and ultimately limits acceptance of cellulose-based renewable bioenergy. With respect to the problem of lignocellulose recalcitrance, it is germane that a number of invertebrate animals, and to some extent, their symbiotic gut fauna, have evolved specialized enzymes that cooperate in lignocellulose processing. In particular, endogenous lignocellulases encoded in marine and terrestrial invertebrate genomes can often confer high degrees of digestion capabilities to these organisms. When endogenous insect lignocellulases work synergistically with symbiont-derived enzymes, this can confer extremely high efficiency in lignocellulose processing. Termites (order Isoptera) are one of the most well recognized examples of an organism that subsists on lignocellulose; and thus, lignocellulase enzymes from termites and their gut symbionts have many potential bioenergy applications that warrant consideration.

Termites are social insects that subsist on sugars and other micronutrients obtained from nutritionally poor lignocellulose diets (Ohkuma M., (2006) Appl. Microbiol. Biotechnol. 61: 1-9; Scharf & Tartar (2008) Biofuels Bioprod Birefin. 2: 540-552). Lignocellulose is a natural complex of the three biopolymers cellulose, hemicellulose and lignin. Cellulose is composed of long β-1.4-linked polymers of glucose that are held together in bundles by hemicellulose (Ljungdahl & Erickson (1985) Adv. Micro. Ecol. 8: 237-299; Lange J. P. (2007) Biofuels Bioprod. Bioref 1: 39-48). Hemicellulose is composed of shorter β-1,4-linked polymers of mixed sugars such as mannose, xylose, galactose, rhamnose, arabinose, glucuronic acid, mannuronic acid, and galacturonic acid (Saha B. C., (2003) J. Indust. Microbiol. Biotechnol. 30: 279-291). Lignin is a 3-dimensional polymer of phenolic compounds that are linked to each other and to hemicellulose by ester bonds. Lignin is composed of three “mono-lignol” monomers (p-coumaryl alcohol, coniferyl alcohol, and sinapyl alcohol), which are combined in different ratios depending on the plant species. Another important characteristic of hemicellulose is its esterification with monomers and dimers of phenolic acid esters, which are identical to the mono-lignols that compose lignin (Saha B. C., (2003) J. Indust. Microbiol. Biotechnol. 30: 279-291; Crepin et al., (2004) Appl. Microbiol. Biotechnol. 63, 647-652; Benoit et al., (2008) Biotechnol. Letters 30, 387-396).

Termites digest lignocellulose with the assistance of endogenous and symbiont-produced digestive enzymes and co-factors (Breznak and Brune (1995) Appl. Env. Microbiol. 61: 2681-2687; Watanabe et al., (1998) Nature 394: 330-331; Ohkuma et al., (2006) Appl. Microbiol. Biotechnol. 61: 1-9; Scharf & Tartar (2008) Biofuels Bioprod. Birefin. 2: 540-552). Termite gut endosymbionts include a diversity of microorganisms that include protozoa, bacteria, spirochetes, fungi, and yeast, among others (Breznak and Brune (1995) Appl. Env. Microbiol. 61: 2681-2687; Warnecke et al., (2007) Nature 450, 560-565). The order Isoptera is divided into the higher and lower termites based mostly on symbiont composition. Lower termites, including Reticulitermes flavipes, possess cellulolytic protozoa in addition to a host of hydrogenic, methanogenic, and nitrogen fixing bacteria and spirochetes. Higher termites lack protozoa altogether, but instead possess cellulolytic bacteria. The roles of endosymbiotic fungi in higher and lower termites are not well defined; however, some higher termites cultivate fungus gardens in their nests that assist in lignocellulose digestion by producing cellulases, hemicellulases and lignases (Taprab et al., (2005) Appl. Env. Microbiol. 71: 7696-7704; Okhuma M., (2006) Appl Microbiol Biotechnol 61, 1-9).

Esterases are hydrolytic enzymes that cleave ester bonds in a diversity of biomolecules (Oakeshott et al. (2005) in Gilbert, L. I., Iatrou, K., Gill, S. S. (eds.) Comprehensive molecular insect science, Vol. 5. Elsevier-Pergamon. New York, pp. 309-382). Some insect esterases have very well defined biological functions, such as those involved in xenobiotic, lipid, acetylcholine, and juvenile hormone metabolism. However, many other insect esterases have largely undefined functions, yet are extremely efficient at metabolizing model substrates such as naphthyl and p-nitrophenyl esters. This latter category of esterases is referred to as the “general esterases”. Because of the highly esterified structure of lignin, it is possible that some general esterases may also contribute to lignin depolymerization in wood feeding insects such as termites.

### SUMMARY

Lignin is an obstacle to the economical production of biofuels from non-food lignocellulose feedstocks. Termites have specialized digestive systems that overcome the lignin barrier in wood to release fermentable simple sugars. The termite gut is thus considered a bioreactor model for enzyme-based production of biofuels from lignocellulose feedstocks. For this reason, using the termite Reticulitermes flavipes and its gut symbionts, high throughput titanium pyrosequencing and proteomics approaches experimentally compared the effects of lignin-containing diets on host-symbiont digestome composition. Over 9,000 distinct host and symbiont transcripts that are differentially expressed in response to diets with varying degrees of lignin complexity, including over 300 responsive cellulase, hemicellulase and candidate lignase transcripts, were identified. Proteomic investigations and functional digestive studies with recombinant lignocellulases conducted in parallel provided strong evidence of congruence at the transcription and translational levels and provide enzymatic strategies for overcoming recalcitrant lignin barriers in biofuel feedstocks.

Briefly described, therefore, one aspect of the disclosure encompasses embodiments of a system for producing a fermentable product from a lignified plant material, the system comprising either: (i) the catalytically active domains, or polypeptides comprising said catalytically active domains, of cellulases Cell-1 and β-glu (beta-glucosidase), and further comprising a catalytically active domain of at least one enzyme, where the enzyme is a cellulase, an aldo-keto-reductase, a catalase, or a laccase; or (ii) the catalytically active domains, or polypeptides comprising said catalytically active domains, of an endo-xylanase and, optionally, a laccase, where the catalytically active domains, or polypeptides comprising said catalytically active domains, can cooperate to provide a fermentable product from a lignified plant material.

In embodiments according to this aspect of the disclosure, at least one of Cell-1, β-glu, a cellulase, an aldo-keto-reductase, a catalase, a laccase, and an endo-xylanase is derived from a termite, or a symbiont thereof.

In embodiments according to this aspect of the disclosure, at least one of the Cell-1, β-glu, a cellulase, an aldo-keto-reductase, a catalase, a laccase, and an endo-xylanase is expressed from a recombinant nucleotide sequence.

In embodiments according to this aspect of the disclosure, the system can comprise a series of isolated recombinant polypeptides, where each polypeptide comprises a catalytically active domain and is expressed from an expression vector of a recombinant expression system. The recombinant expression system can be selected from a eukaryotic cell-based system and a prokaryotic cell-based system.

In embodiments of the system according to this aspect of the disclosure, the expression vector can be a baculovirus expression vector and the recombinant expression system is a eukaryotic cell-based system.

In embodiments of the system according to this aspect of the disclosure, the catalytically active domains, or polypeptides comprising said catalytically active domains, can cooperate to provide a sugar from a lignified plant material.

In embodiments of the system according to this aspect of the disclosure, the catalytically active domains can cooperate to provide glucose from a lignified plant material.

In embodiments of the system according to this aspect of the disclosure, the catalytically active domains, or polypeptides comprising said catalytically active domains, can cooperate to provide a pentose from a lignified plant material.

In embodiments of the system according to this aspect of the disclosure, catalytically active domains, or polypeptides comprising said catalytically active domains, can be Cell-1 and β-glu, and, additionally, at least one cellulase GHF7-3, aldo-keto-reductase, catalase, or laccase.

In embodiments of the system according to this aspect of the disclosure, the system can comprise the catalytically active domains, or polypeptides comprising said catalytically active domains, of Cell-1, β-glu, and cellulase GHF7-3, and either an aldo-keto-reductase or a catalase.

In embodiments of the system according to this aspect of the disclosure, the system can comprise the catalytically active domains, or polypeptides comprising said catalytically active domains, of an endo-xylanase and a laccase. The laccase can be LacA.

In embodiments of the system according to this aspect of the disclosure, the system can comprise the catalytically active domains, or polypeptides comprising said catalytically active domains, of: (i) Cell-1, β-glu, cellulase GHF7-3, LacA, aldo-keto-reductase, and a catalase; (ii) Cell-1, β-glu, cellulase GHF7-3, and a catalase; (iii) Cell-1, β-glu, cellulase GHF7-3, and aldo-keto-reductase; (iv) Cell-1, β-glu, cellulase GHF7-3, and LacA; (v) Cell-1, β-glu, and cellulase GHF7-3; (vi) Cell-1, β-glu, and a catalase; (vii) Cell-1, β-glu, and aldo-keto-reductase; or (viii) LacA AND GHF11-1.

Another aspect of the disclosure encompasses embodiments of an expression vector encoding a polypeptide, or a catalytically active variant thereof, of Cell-1, β-glu, cellulase GHF7-3, LacA, aldo-keto-reductase, a catalase, or GHF11-1. In embodiments of this aspect of the disclosure the expression vector can be in a host cell or tissue. In some embodiments of this aspect of the disclosure, the expression vector can be derived from a baculovirus.

Another aspect of the disclosure encompasses embodiments of a method of converting a lignified plant material to a fermentable product, the method comprising the steps of: (a) obtaining a system of catalytically active domains, or polypeptides comprising said catalytically active domains, according to any of the above paragraphs; and (b) incubating the system with a source of lignified plant material under conditions allowing the polypeptides to cooperatively produce a fermentable product from the lignified plant material.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1A schematically illustrates a termite gut. SG, salivary gland; FG, Foregut; MG, midgut; MT, Malpighian tubules; HG, hindgut. Cellulolytic symbionts reside mainly in the HG region account for about ⅔ of lignocellulose digestion; whereas, host tissues (SG. FG and MG) account for about ⅓.

FIG. 1B shows a table illustrating a summary of pyrosequencing data.

FIG. 1C illustrates a Venn diagram showing sequence distributions among the cellulose-subtracted wood and lignin libraries.

FIG. 2 is a bar graph illustrating glucose release from pine wood lignocellulose by recombinant enzyme cocktails. Bars represent micromoles of glucose released per minute (±std. error) for various combinations of recombinant enzymes encoded by differentially expressed genes identified in the present study. Six recombinant enzymes were tested: Cell-1, β-glu, GHF7-3, LacA, AKR and CAT. The first three enzymes are cellulases from Glycosyl Hydrolase Families (GHF) 9, 1 and 7; the latter three are lignase/phenoloxidase candidates from the laccase, aldo-keto reductase, and catalase families, respectively. All incubations lasted 18 hr.

FIG. 3 is a bar graph illustrating xylose release from pine lignocellulose by recombinant GHF11-1 xylanase and LacA laccase enzyme cocktails. Bars represent micromoles of xylose released per minute (±std. error) for three treatments: (1) GHF11-1 alone, (2) co-incubation of GHF11-1+LacA, and (3) incubation with LacA for 4 hr before addition of GHF11-1. All GHF11-1 incubations lasted 18 hr.

FIG. 4 schematically illustrates an experimental design flow chart over-viewing bioassay, molecular biology and bioinformatic procedures.

FIGS. 5A-5C are graphs illustrating sequence similarity distributions for the wood (FIG. 5A), lignin (FIG. 5B), and combined (wood+lignin) (FIG. 5C) datasets.

FIGS. 6A-6C are a series of graphs illustrating E-value distributions for the wood (FIG. 6A), lignin (FIG. 6B) and combined (wood+lignin) (FIG. 6C) datasets.

FIGS. 7A-7D are a series of graphs illustrating BLASTx results distributions for the wood (FIG. 7A), lignin (FIG. 7B) and combined (wood+lignin) (FIG. 7C) datasets.

FIG. 8A is a digital image of a two-dimensional PAGE analysis of soluble termite gut proteins from workers termites fed a diet of paper+lignin alkali. Highlighted spots were those chosen for analysis by tandem MS (n=26). Approximately 35 kDa protein spots were identified as being the most highly up-regulated in association with lignin alkali feeding.

FIG. 8B illustrates the amino acid sequences of aldo-keto reductase (AKR) sequenced peptides (spots 1820, 1829, 1834).

FIG. 9 illustrates the nucleotide sequence of the assembled Aldo-keto reductase nucleotide contig, which represents an apparent full-length cDNA sequence (1737 nucleotides). Two candidate start codons (ATG) are underlined, as well as the stop codon TAA, polyadenylation signal (AATAA) and poly-A tail (AAAAAₙ).

FIG. 10 illustrates the translated AKR cDNA sequence and peptides sequenced by tandem MS. Nucleotides are shown in lower case letters and amino acid translations are shown above nucleotides in capital letters. Gray highlighting indicates peptide sequences obtained in the current proteomics work. Black highlighting indicates potential amino acid glycosylation sites. Dotted underlining indicates a putative signal peptide sequence (possible signal cleavage site indicated by “▾”). Two putative ATG start codons and the TAA stop codon are show by underlined and bold font, as well as polyadenylation sites (AAATAAA) and poly A tails (AAAAAAAA).

FIG. 11 is a digital image illustrating filter paper feeding by groups of 50 worker termites over 7-day assays (left: paper alone; right: paper+lignin alkali).

FIG. 12 is a digital image illustrating a one-dimensional SDS-PAGE analysis (10% acrylamide, 1% SDS) of different gut protein fractions with GEL-CODE BLUE® staining. MW: molecular weight markers; P1: 1,000×g nuclear pellet; P2: 10,000×g mitochondrial pellet; P3: microsomal pellet precipitated at 10,000×g in the presence of 8 mM calcium chloride); Soluble: soluble fraction remaining after precipitation of P1, P2 and P3 pellets; kDa: kilodaltons; ←: differentially expressed approximately 35 kDa protein band in the soluble lignin alkali fraction.

FIG. 13 is a digital image illustrating a two dimensional separation of the soluble gut protein fraction with two-color imaging to show differentially expressed proteins. The strongest lignin alkali-up-regulated proteins are enclosed in the box

FIG. 14A illustrates the synthetic nucleotide sequence, codon-optimized for the host Trichoplusia ni, of catalase.

FIG. 14B illustrates the recombinant protein sequence of catalase. The mature protein sequence of the catalase lacks the first Met.

FIG. 15A illustrates the native nucleotide sequence of Aldo-keto reductase (AKR). Positions of ATG start codons and TAA termination codon are in bold.

FIG. 15B illustrates the synthetic nucleotide sequence, codon-optimized for the host Trichoplusia ni, of aldo-keto reductase (AKR).

FIG. 15C illustrates the recombinant protein sequence and the mature protein sequence of aldo-keto reductase (AKR).

FIG. 16 illustrates the nucleotide sequence of catalase (CAT).

FIG. 17 illustrates the nucleotide sequence of GHF7-3 cellulase.

FIG. 18A illustrates the synthetic nucleotide sequence, codon-optimized for the host Trichoplusia ni, of GHF11-1 hemicellulase.

FIG. 18B illustrates the recombinant protein sequence and the mature protein sequence of GHF11-1 hemicellulase.

FIG. 19 illustrates the nucleotide sequence of LacA laccase. Positions of ATG start codons and TAA termination codon are in bold and the positions of PCR primers used for insertion into a baculovirus expression vector are underlined.

FIG. 20A illustrates the cloning strategy and the nucleotide sequence of GHF9 Cell-1 cellulase.

FIG. 20B illustrates the recombinant protein sequence and the mature protein sequence of GHF9 Cell-1 cellulase.

FIG. 21A illustrates the cloning strategy and full-length nucleotide sequence of β-glu cellulase.

FIG. 21B illustrates the recombinant protein sequence and the mature protein sequence of β-glu cellulase.

FIG. 22 illustrates the nucleotide sequence encoding GHF7-3 encompassing the region PCR amplified for cloning into the baculovirus expression vector. The natural ATG start codon, TAG stop codon, and the forward and reverse PCR primer positions are indicated in bold and underlining.

FIG. 23 illustrates the amino acid sequence of the recombinant GHF7-3 protein sequence with a leader sequence and the thrombin-cleavable (His)6 terminus sequence GTLVPRGSHHHHHH.

FIG. 24 illustrates the amino acid sequence of the recombinant GHF7-3 mature protein sequence.

FIG. 25 illustrates the recombinant protein sequence and the mature protein sequence of Laccase 6.

FIG. 26 illustrates the recombinant protein sequence and the mature protein sequence of Laccase 12.

FIG. 27 illustrates glucose release from pine wood lignocellulose by termite recombinant enzyme cocktails. Bars represent micromoles of glucose released per minute (±std. error) for various combinations of recombinant enzymes. Seven recombinant enzymes were tested: GHF9 endoglucanase (Cell-1), GHF1 beta-glucosidase (β-glu), GHF7 exoglucanase (GHF7-3), superoxide dismutase (SOD), glutathione peroxidase (GPx), catalase (CAT), and laccase (LacA). All incubations lasted 18 hr.

FIG. 28 illustrates the release of reducing sugars from pine wood lignocellulose by termite recombinant enzyme cocktails is depicted. Bars represent micromoles of reducing sugars released per minute (±std. error) for various combinations of recombinant enzymes. Seven recombinant enzymes were tested: GHF9 endoglucanase (Cell-1), GHF1 beta-glucosidase (β-glu), GHF7 exoglucanase (GHF7-3), superoxide dismutase (SOD), glutathione peroxidase (GPx), aldo-keto reductase (AKR), and catalase (CAT). Glutathione (GSH), a cofactor required for GPx activity, was added in the cocktails with GPx enzyme. All incubations lasted 18 hr.

FIGS. 29A-29C illustrate glucose release from pine wood lignocellulose by various combinations of Trichoderma reesei cellulase (TrC; Celluclast®, Novozyme Corp.) and termite cellulase enzymes. Bars represent micromoles of glucose released per minute (±std. error). Twelve serial dilutions (from 4000-2 μg) of TrC were tested with 8 g fixed concentration of termite recombinant enzymes: GHF9 endoglucanase (Cell-1). GHF1 beta-glucosidase (β-glu), and GHF7 exoglucanase (GHF7-3). All incubations lasted 18 hr.

FIGS. 30A-30D illustrate glucose release from pine wood lignocellulose by various combinations of Trichoderma reesei cellulase (TrC; Celluclast®, Novozyme Corp.) and termite lignase/detox enzymes is depicted. Bars represent micromoles of glucose released per minute (±std. error). Twelve serial dilutions (from 4000-2 μg) of TrC were tested with 81 μg fixed concentration of termite recombinant enzymes: laccase (LacA), aldo-keto reductase (AKR), catalase (CAT), and superoxide dismutase (SOD). All incubations lasted 18 hr.

FIG. 31 illustrates reducing sugars release from pine wood lignocellulose by various combinations of Trichoderma reesei cellulase (TrC; Celluclast®, Novozyme Corp.) and termite glutathione peroxidase (GPx) enzyme is depicted. Bars represent micromoles of reducing sugars released per minute (±std. error). Twelve serial dilutions (from 4000-2 μg) of TrC were tested with 8 μg fixed concentration of GPx enzyme. Glutathione (6 mM), a cofactor required for GPx activity, was added in the cocktails. All incubations lasted 18 hr.

FIGS. 32A-32C illustrate the assay conditions standardized for two recombinant enzymes: glutathione peroxidase (GPx) and superoxide dismutase (SOD). Crude homogenate and clarified supernatant of both the enzymes were tested for their enzymatic activities.

The details of some exemplary embodiments of the methods and systems of the present disclosure are set forth in the description below. Other features, objects, and advantages of the disclosure will be apparent to one of skill in the art upon examination of the following description, drawings, examples and embodiments. It is intended that all such additional systems, methods, features, and advantages be included within this description, be within the scope of the present disclosure.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended embodiments, the singular forms “a,” “an,” and “the” include plural referents unless the context clearly dictates otherwise. Thus, for example, reference to “a support” includes a plurality of supports. In this specification and in the embodiments that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. Patent law and can mean “includes.” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

In describing the disclosed subject matter, the following terminology will be used in accordance with the definitions set forth below.

The term “termite gut” as used herein refers to the gut of R. flavipes workers. The gut of R. flavipes workers is composed of three main regions: foregut, midgut, and hindgut. The foregut region includes the esophagus, crop, and attached salivary gland. The salivary glands secrete endogenous (termite-derived) digestive factors and enzymes into the digestive tract. The midgut is a slender, tubular region that secretes a peritrophic matrix around food materials and, presumably, is a location where some lignocellulose degradation occurs. The Malpighian tubules connect at the junction of the midgut and hindgut and participate in waste excretion. The hindgut includes a fermentation chamber that is generally anaerobic in its core, but it does possess a micro-oxic zone around its periphery. The hindgut houses gut symbionts, and it is the location where most lignocellulose degradation, as well as fermentation and nutrient assimilation, are thought to occur.

The fermentation chamber of the hindgut is a source of microbial diversity. Microorganisms from various taxa present in the termite gut include bacteria/spirochetes and protozoans. In lower termites such as R. flavipes, protozoan symbionts are considered to be primarily involved in cellulose/hemicellulose degradation, while bacteria are considered important to nitrogen economy and simple sugar fermentation. Spirochetes, which are difficult to culture, are found in the hindguts of all termites. Spirochetes play roles in acetogenesis and nitrogen fixation, and they and other endomicrobionts also occur as cytoplasmic symbionts of hindgut protozoa.

The term “lignocellulose” as used herein refers to a natural complex of the three biopolymers: cellulose, hemicellulose and lignin. Cellulose is composed of rigid, high-molecular-weight, β-1,4-linked polymers of glucose that are held together in bundles by hemicellulose. Hemicellulose is composed of shorter β-1,4-linked polymers of mixed sugars. Mannose is usually the dominant sugar present in hemicelluloses of softwoods fed upon by termites, with lesser amounts of xylose, galactose, rhamnose, arabinose, glucuronic acid, mannuronic acid and galacturonic acid.

The term “lignin” as used herein refers to a 3-dimensional polymer of phenolic compounds that are linked to each other and to hemicellulose by ester bonds. Lignin is composed of the three mono-lignol monomers p-coumaryl alcohol, coniferyl alcohol, and sinapyl alcohol combined in different ratios depending on the plant species. Another noteworthy aspect of hemicellulose is its high degree of esterification with monomers and dimers of phenolic acid esters, which are analogous to the mono-lignols noted above. Phenolic acid esters are derived mostly from the mono-lignols p-coumaryl and coniferyl alcohol (i.e., coumaric acid and ferulic acid). The three individual lignocellulose components, cellulose, hemicellulose and lignin, compose approximately 40%, 25%, and 20%, respectively, of lignocellulose (Lange 2007).

The term “pentose” as used herein refers to a monosaccharide with five carbon atoms. Pentoses are organized into two groups: aldopentoses having an aldehyde functional group at position 1; and ketopentoses have a ketone functional group in position 2 or 3. The aldopentoses have three chiral centers and therefore eight different stereoisomers are possible and include: arabinose, xylose, and ribose. Ketopentoses have two chiral centers, and therefore four different stereoisomers are possible, and include ribulose and xylulose. The term “pentose” as used herein refers to rhamnose, a naturally occurring deoxy sugar classified as a methyl-pentose or a 6-deoxy-hexose. Rhamnose occurs in nature in its L-form as L-rhamnose.

The term “catalase” as used herein refers to an enzyme that catalyzes the decomposition of hydrogen peroxide to water and oxygen. Catalase has one of the highest turnover numbers of all enzymes; one catalase molecule can convert millions of molecules of proteins, which contains a hydrogen peroxide to water and oxygen each second. Catalase is a tetramer of four polypeptide chains, each over 500 amino acids long. It contains four porphyrin heme (iron) groups that allow the enzyme to react with the hydrogen peroxide.

The term “aldo-keto reductase” as used herein refers to a family of enzymes that includes a number of related monomeric NADPH-dependent oxidoreductases, such as aldehyde reductase, aldose reductase, prostaglandin F synthase, xylose reductase, rho crystallin, and the like. All possess a similar structure, with a beta-alpha-beta fold characteristic of nucleotide binding novel NADP-binding motif. The hydrophobic nature of the pocket favors aromatic and apolar substrates over highly polar ones. Binding of the NADPH coenzyme causes a massive conformational change, reorienting a loop, effectively locking the coenzyme in place. This binding is more similar to FAD- than to NAD(P)-binding oxidoreductases.

The term “xylanase” as used herein refers to a class of enzymes which degrade the linear polysaccharide beta-1,4-xylan into xylose, thus breaking down hemicellulose, one of the major components of plant cell walls. As such, it plays a major role in micro-organisms thriving on plant sources. Xylanases are present in fungi for the degradation of plant matter into usable nutrients.

The term “catalytically active domain” as used herein refers to an isolated region of an enzyme that retains the catalytic activity of the enzyme polypeptide found in the native cell. The size of the domain can vary according to the enzyme and the need to retain amino acid sequences that allow or maintain the three-dimensional structure of the enzymatically-active domain.

The term “nucleic acid” as used herein refers to any natural and synthetic linear and sequential arrays of nucleotides and nucleosides, for example cDNA, genomic DNA, mRNA, tRNA, oligonucleotides, oligonucleosides and derivatives thereof. For ease of discussion, such nucleic acids may be collectively referred to herein as “constructs,” “plasmids,” or “vectors.” Representative examples of the nucleic acids of the present disclosure include bacterial plasmid vectors including expression, cloning, cosmid and transformation vectors such as, but not limited to, pBR322, animal viral vectors such as, but not limited to, modified adenovirus, influenza virus, polio virus, pox virus, retrovirus, insect viruses (baculovirus), and the like, vectors derived from bacteriophage nucleic acid, and synthetic oligonucleotides like chemically synthesized DNA or RNA. The term “nucleic acid” further includes modified or derivatized nucleotides and nucleosides such as, but not limited to, halogenated nucleotides such as, but not only, 5-bromouracil, and derivatized nucleotides such as biotin-labeled nucleotides.

The term “isolated nucleic acid” as used herein refers to a nucleic acid with a structure (a) not identical to that of any naturally occurring nucleic acid or (b) not identical to that of any fragment of a naturally occurring genomic nucleic acid spanning more than three separate genes, and includes DNA, RNA, or derivatives or variants thereof. The term covers, for example, (a) a DNA which has the sequence of part of a naturally occurring genomic molecule but is not flanked by at least one of the coding sequences that flank that part of the molecule in the genome of the species in which it naturally occurs: (b) a nucleic acid incorporated into a vector or into the genomic nucleic acid of a prokaryote or eukaryote in a manner such that the resulting molecule is not identical to any vector or naturally occurring genomic DNA; (c) a separate molecule such as a cDNA, a genomic fragment, a fragment produced by polymerase chain reaction (PCR), ligase chain reaction (LCR) or chemical synthesis, or a restriction fragment; (d) a recombinant nucleotide sequence that is part of a hybrid gene. i.e., a gene encoding a fusion protein, and (e) a recombinant nucleotide sequence that is part of a hybrid sequence that is not naturally occurring. Isolated nucleic acid molecules of the present disclosure can include, for example, natural allelic variants as well as nucleic acid molecules modified by nucleotide deletions, insertions, inversions, or substitutions.

The term “enriched” as used herein in reference to nucleic acid is meant that the specific DNA or RNA sequence constitutes a significantly higher fraction of the total DNA or RNA present in the cells or solution of interest than in normal or diseased cells or in the cells from which the sequence was taken. Enriched does not imply that there are no other DNA or RNA sequences present, just that the relative amount of the sequence of interest has been significantly increased. The other DNA may, for example, be derived from a yeast or bacterial genome, or a cloning vector, such as a plasmid or a viral vector. The term “significant” as used herein is used to indicate that the level of increase is useful to the person making such an increase.

It is advantageous for some purposes that a nucleotide sequence is in purified form. The term “purified” in reference to nucleic acid represents that the sequence has increased purity relative to the natural environment.

The terms “polynucleotide,” “oligonucleotide,” and “nucleic acid sequence” are used interchangeably herein and include, but are not limited to, coding sequences (polynucleotide(s) or nucleic acid sequence(s) which are transcribed and translated into polypeptide in vitro or in vivo when placed under the control of appropriate regulatory or control sequences); control sequences (e.g., translational start and stop codons, promoter sequences, ribosome binding sites, polyadenylation signals, transcription factor binding sites, transcription termination sequences, upstream and downstream regulatory domains, enhancers, silencers, and the like); and regulatory sequences (DNA sequences to which a transcription factor(s) binds and alters the activity of a gene's promoter either positively (induction) or negatively (repression)). No limitation as to length or to synthetic origin is suggested by the terms described herein.

The terms “polypeptide” and “protein” as used herein refer to a polymer of amino acids of three or more amino acids in a serial array, linked through peptide bonds. The term “polypeptide” includes proteins, protein fragments, protein analogues, oligopeptides and the like. The term “polypeptides” contemplates polypeptides as defined above that are encoded by nucleic acids, produced through recombinant technology (isolated from an appropriate source such as a bird), or synthesized. The term “polypeptides” further contemplates polypeptides as defined above that include chemically modified amino acids or amino acids covalently or non-covalently linked to labeling ligands.

The term “fragment” as used herein to refer to a nucleic acid (e.g., cDNA) refers to an isolated portion of the subject nucleic acid constructed artificially (e.g., by chemical synthesis) or by cleaving a natural product into multiple pieces, using restriction endonucleases or mechanical shearing, or a portion of a nucleic acid synthesized by PCR, DNA polymerase or any other polymerizing technique well known in the art, or expressed in a host cell by recombinant nucleic acid technology well known to one of skill in the art. The term “fragment” as used herein may also refer to an isolated portion of a polypeptide, wherein the portion of the polypeptide is cleaved from a naturally occurring polypeptide by proteolytic cleavage by at least one protease, or is a portion of the naturally occurring polypeptide synthesized by chemical methods well known to one of skill in the art.

The term “gene” or “genes” as used herein refers to nucleic acid sequences (including both RNA and DNA) that encode genetic information for the synthesis of a whole RNA, a whole protein, or any portion of such whole RNA or whole protein. Genes that are not naturally part of a particular organism's genome are referred to as “foreign genes,” “heterologous genes” or “exogenous genes” and genes that are naturally a part of a particular organism's genome are referred to as “endogenous genes”. The term “gene product” refers to RNAs or proteins that are encoded by the gene. “Foreign gene products” are RNA or proteins encoded by “foreign genes” and “endogenous gene products” are RNA or proteins encoded by endogenous genes. “Heterologous gene products” are RNAs or proteins encoded by “foreign, heterologous or exogenous genes” and are, therefore, not naturally expressed in the cell.

The term “expressed” or “expression” as used herein refers to the transcription from a gene to give an RNA nucleic acid molecule at least complementary in part to a region of one of the two nucleic acid strands of the gene. The term “expressed” or “expression” as used herein also refers to the translation from said RNA nucleic acid molecule to give a protein, a polypeptide, or a portion or fragment thereof.

The term “operably linked” refers to an arrangement of elements wherein the components so described are configured so as to perform their usual function. Control sequences operably linked to a coding sequence are capable of effecting the expression of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

The term “cooperate to provide” as used herein refers to at least two enzymes, or functional fragments thereof that convert a substrate compound to a product compound by a series of reactions catalyzed by the enzymes or fragments thereof. In cooperating, it is contemplated that the enzymes or functional fragments thereof may be physically associated as a single poly peptide expressed from a single nucleotide sequence, as a complex of the at least two polypeptides, a system wherein the enzymatically active polypeptides are not in association with one another, or partially so.

The terms “transcription regulatory sequences” and “gene expression control regions” as used herein refer to nucleotide sequences that are associated with a gene nucleic acid sequence and which regulate the transcriptional expression of the gene. Exemplary transcription regulatory sequences include enhancer elements, hormone response elements, steroid response elements, negative regulatory elements, and the like. The “transcription regulatory sequences” may be isolated and incorporated into a vector nucleic acid to enable regulated transcription in appropriate cells of portions of the vector DNA. The “transcription regulatory sequence” may precede, but is not limited to, the region of a nucleic acid sequence that is in the region 5′ of the end of a protein coding sequence that may be transcribed into mRNA. Transcriptional regulatory sequences may also be located within a protein coding region, in regions of a gene that are identified as “intron” regions, or may be in regions of nucleic acid sequence that are in the region of nucleic acid.

The term “promoter” as used herein refers to the DNA sequence that determines the site of transcription initiation from an RNA polymerase. A “promoter-proximal element” may be a regulatory sequence within about 200 base pairs of the transcription start site.

The term “coding region” as used herein refers to a continuous linear arrangement of nucleotides that may be translated into a protein. A full length coding region is translated into a full length protein (a complete protein as would be translated in its natural state absent any post-translational modifications). A full length coding region may also include any leader protein sequence or any other region of the protein that may be excised naturally from the translated protein.

The term “complementary” as used herein refers to two nucleic acid molecules that can form specific interactions with one another. In the specific interactions, an adenine base within one strand of a nucleic acid can form two hydrogen bonds with thymine within a second nucleic acid strand when the two nucleic acid strands are in opposing polarities. Also in the specific interactions, a guanine base within one strand of a nucleic acid can form three hydrogen bonds with cytosine within a second nucleic acid strand when the two nucleic acid strands are in opposing polarities. Complementary nucleic acids as referred to herein, may further comprise modified bases wherein a modified adenine may form hydrogen bonds with a thymine or modified thymine, and a modified cytosine may form hydrogen bonds with a guanine or a modified guanine.

The term “probe” as used herein, when referring to a nucleic acid, refers to a nucleotide sequence that can be used to hybridize with and thereby identify the presence of a complementary sequence, or a complementary sequence differing from the probe sequence but not to a degree that prevents hybridization under the hybridization stringency conditions used. The probe may be modified with labels such as, but not only, radioactive groups, chemiluminescent moieties, biotin, and the like that are well known in the art.

The terms “unique nucleic acid region” and “unique protein (polypeptide) region” as used herein refer to sequences present in a nucleic acid or protein (polypeptide) respectively that is not present in any other nucleic acid or protein sequence. The terms “conserved nucleic acid region” as referred to herein is a nucleotide sequence present in two or more nucleic acid sequences, to which a particular nucleic acid sequence can hybridize under low, medium or high stringency conditions. The greater the degree of conservation between the conserved regions of two or more nucleic acid sequences, the higher the hybridization stringency that will allow hybridization between the conserved region and a particular nucleic acid sequence.

The term “sense strand” as used herein refers to a single stranded DNA molecule from a genomic DNA that may be transcribed into RNA and translated into the natural polypeptide product of the gene. The term “antisense strand” as used herein refers to the single strand DNA molecule of a genomic DNA that is complementary with the sense strand of the gene.

The term “nucleic acid vector” as used herein refers to a natural or synthetic single or double stranded plasmid or viral nucleic acid molecule that can be transfected or transformed into cells and replicate independently of, or within, the host cell genome. A circular double stranded plasmid can be linearized by treatment with an appropriate restriction enzyme based on the nucleotide sequence of the plasmid vector. A nucleic acid can be inserted into a vector by cutting the vector with restriction enzymes and ligating the pieces together. The nucleic acid molecule can be RNA or DNA.

The term “expression vector” as used herein refers to a nucleic acid vector that comprises a gene expression control region operably linked to a nucleotide sequence coding at least one polypeptide. As used herein, the term “regulatory sequences” includes promoters, enhancers, and other elements that may control gene expression. Standard molecular biology textbooks (for example, Sambrook et al., eds., 1989, “Molecular Cloning: A Laboratory Manual,” 2nd ed., Cold Spring Harbor Press) may be consulted to design suitable expression vectors that may further include an origin of replication and selectable gene markers. It should be recognized, however, that the choice of a suitable expression vector and the combination of functional elements therein depends upon multiple factors including the choice of the host cell to be transformed and/or the type of protein to be expressed.

The terms “transformation” and “transfection” as used herein refer to the process of inserting a nucleic acid into a host. Many techniques are well known to those skilled in the art to facilitate transformation or transfection of a nucleic acid into a prokaryotic or eukaryotic organism. These methods involve a variety of techniques, such as treating the cells with high concentrations of salt such as, but not only, a calcium or magnesium salt, an electric field, detergent, or liposome mediated transfection, to render the host cell competent for the uptake of the nucleic acid molecules, and by such methods as sperm-mediated and restriction-mediated integration.

The term “transfecting agent” as used herein refers to a composition of matter added to the genetic material for enhancing the uptake of heterologous DNA segment(s) into a eukaryotic cell including, but not limited to, an insect host cell. The enhancement is measured relative to the uptake in the absence of the transfecting agent. Examples of transfecting agents include adenovirus-transferrin-polylysine-DNA complexes. These complexes generally augment the uptake of DNA into the cell and reduce its breakdown during its passage through the cytoplasm to the nucleus of the cell. Other preferred transfecting agents include, but are not limited to, lipofectin, lipofectamine, DIMRIE C, Supeffect, and Effectin (Qiagen), unifectin, maxifectin, DOTMA, DOGS (Transfectam; dioctadecylamidoglycylspermine), DOPE (1,2-dioleoyl-sn-glycero-3-phosphoethanolamine), DOTAP (1,2-dioleoyl-3-trimethylammonium propane), DDAB (dimethyl dioctadecytammonium bromide), DHDEAB (N,N-di-n-hexadecyl-N,N-dihydroxyethyl ammonium bromide), HDEAB (N-n-hexadecylN,N-dihydroxyethylammonium bromide), polybrene, poly(ethylenimine) (PEI) and the like.

The term “recombinant cell” refers to a cell that has a new combination of nucleic acid segments that are not covalently linked to each other in nature. A new combination of nucleic acid segments can be introduced into an organism using a wide array of nucleic acid manipulation techniques available to those skilled in the art. A recombinant cell can be a single eukaryotic cell, or a single prokaryotic cell, or a mammalian cell. The recombinant cell may harbor a vector that is extragenomic. An extragenomic nucleic acid vector does not insert into the cell's genome. A recombinant cell may further harbor a vector or a portion thereof that is intragenomic. The term intragenomic defines a nucleic acid construct incorporated within the recombinant cell's genome.

The terms “recombinant nucleic acid” and “recombinant DNA” as used herein refer to combinations of at least two nucleic acid sequences that are not naturally found in a eukaryotic or prokaryotic cell. The nucleic acid sequences include, but are not limited to, nucleic acid vectors, gene expression regulatory elements, origins of replication, suitable gene sequences that when expressed confer antibiotic resistance, protein-encoding sequences, and the like. The term “recombinant polypeptide” is meant to include a polypeptide produced by recombinant DNA techniques such that it is distinct from a naturally occurring polypeptide either in its location, purity or structure. Generally, such a recombinant polypeptide will be present in a cell in an amount different from that normally observed in nature.

The techniques used to isolate and characterize the nucleic acids and proteins of the present disclosure are well known to those of skill in the art and standard molecular biology and biochemical manuals may be consulted to select suitable protocols without undue experimentation (see, for example. Sambrook et al., “Molecular Cloning: A Laboratory Manual,” 2nd ed., 1989. Cold Spring Harbor Press; the contents of which is incorporated herein by reference in its entirety).

A “cyclic polymerase-mediated reaction” refers to a biochemical reaction in which a template molecule or a population of template molecules is periodically and repeatedly copied to create a complementary template molecule or complementary template molecules, thereby increasing the number of the template molecules over time.

“Denaturation” of a template molecule refers to the unfolding or other alteration of the structure of a template so as to make the template accessible to duplication. In the case of DNA, “denaturation” refers to the separation of the two complementary strands of the double helix, thereby creating two complementary, single stranded template molecules. “Denaturation” can be accomplished in any of a variety of ways, including by heat or by treatment of the DNA with a base or other denaturant.

“DNA amplification” as used herein refers to any process that increases the number of copies of a specific DNA sequence by enzymatically amplifying the nucleic acid sequence. A variety of processes are known. One of the most commonly used is the polymerase chain reaction (PCR), which is defined and described in later sections below. The PCR process of Mullis is described in U.S. Pat. Nos. 4,683,195 and 4,683,202. PCR involves the use of a thermostable DNA polymerase, known sequences as primers, and heating cycles, which separate the replicating deoxyribonucleic acid (DNA), strands and exponentially amplify a gene of interest. Any type of PCR, such as quantitative PCR, RT-PCR, hot start PCR. LAPCR, multiplex PCR, touchdown PCR, etc., may be used. Advantageously, real-time PCR is used. In general, the PCR amplification process involves an enzymatic chain reaction for preparing exponential quantities of a specific nucleic acid sequence. It requires a small amount of a sequence to initiate the chain reaction and oligonucleotide primers that will hybridize to the sequence. In PCR the primers are annealed to denatured nucleic acid followed by extension with an inducing agent (enzyme) and nucleotides. This results in newly synthesized extension products. Since these newly synthesized sequences become templates for the primers, repeated cycles of denaturing, primer annealing, and extension results in exponential accumulation of the specific sequence being amplified. The extension product of the chain reaction will be a discrete nucleic acid duplex with a termini corresponding to the ends of the specific primers employed.

“DNA” refers to the polymeric form of deoxyribonucleotides (adenine, guanine, thymine, or cytosine) in either single stranded form, or as a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA).

By the terms “enzymatically amplify” or “amplify” is meant, for the purposes of the specification or embodiments. DNA amplification, i.e., a process by which nucleic acid sequences are amplified in number. There are several means for enzymatically amplifying nucleic acid sequences. Currently the most commonly used method is the polymerase chain reaction (PCR). Other amplification methods include LCR (ligase chain reaction) which utilizes DNA ligase, and a probe consisting of two halves of a DNA segment that is complementary to the sequence of the DNA to be amplified, enzyme Qβ replicase and a ribonucleic acid (RNA) sequence template attached to a probe complementary to the DNA to be copied which is used to make a DNA template for exponential production of complementary RNA; strand displacement amplification (SDA); Qβ replicase amplification (QβRA); self-sustained replication (3SR); and NASBA (nucleic acid sequence-based amplification), which can be performed on RNA or DNA as the nucleic acid sequence to be amplified.

As used herein, the term “genome” refers to all the genetic material in the chromosomes of a particular organism. Its size is generally given as its total number of base pairs. Within the genome, the term “gene” refers to an ordered sequence of nucleotides located in a particular position on a particular chromosome that encodes a specific functional product (e.g., a protein or RNA molecule). In general, a patient's genetic characteristics, as defined by the nucleotide sequence of its genome, are known as its “genotype.” while the patient's physical traits are described as its “phenotype.”

The term “polymerase chain reaction” or “PCR” refers to a thermocyclic, polymerase-mediated, DNA amplification reaction. A PCR typically includes template molecules, oligonucleotide primers complementary to each strand of the template molecules, a thermostable DNA polymerase, and deoxyribonucleotides, and involves three distinct processes that are multiply repeated to effect the amplification of the original nucleic acid. The three processes (denaturation, hybridization, and primer extension) are often performed at distinct temperatures, and in distinct temporal steps. In many embodiments, however, the hybridization and primer extension processes can be performed concurrently. The nucleotide sample to be analyzed may be PCR amplification products provided using the rapid cycling techniques described in U.S. Pat. Nos. 6,569,672; 6,569,627; 6,562,298; 6,556,940; 6,569,672; 6,569,627; 6,562,298; 6,556,940; 6,489,112; 6,482,615; 6,472,156; 6,413,766; 6,387,621; 6,300,124; 6,270,723; 6,245,514; 6,232,079; 6,228,634; 6,218,193; 6,210,882; 6,197,520; 6,174,670; 6,132,996; 6,126,899; 6,124,138; 6,074,868; 6,036,923; 5,985,651; 5,958,763; 5,942,432; 5,935,522; 5,897,842; 5,882,918; 5,840,573; 5,795,784; 5,795,547; 5,785,926; 5,783,439; 5,736,106; 5,720,923; 5,720,406; 5,675,700; 5,616,301; 5,576,218 and 5,455,175, the disclosures of which are incorporated by reference in their entireties. Other methods of amplification include, without limitation, NASBR, SDA, 3SR, TSA and rolling circle replication. It is understood that, in any method for producing a polynucleotide containing given modified nucleotides, one or several polymerases or amplification methods may be used. The selection of optimal polymerization conditions depends on the application.

A “polymerase” is an enzyme that catalyzes the sequential addition of monomeric units to a polymeric chain, or links two or more monomeric units to initiate a polymeric chain. In advantageous embodiments of this disclosure, the “polymerase” will work by adding monomeric units whose identity is determined by and which is complementary to a template molecule of a specific sequence. For example, DNA polymerases such as DNA pol 1 and Taq polymerase add deoxyaribonucleotides to the 3′ end of a polynucleotide chain in a template-dependent manner, thereby synthesizing a nucleic acid that is complementary to the template molecule. Polymerases may be used either to extend a primer once or repetitively or to amplify a polynucleotide by repetitive priming of two complementary strands using two primers.

A “primer” is an oligonucleotide, the sequence of at least a portion of which is complementary to a segment of a template DNA which to be amplified or replicated. Typically primers are used in performing the polymerase chain reaction (PCR). A primer hybridizes with (or “anneals” to) the template DNA and is used by the polymerase enzyme as the starting point for the replication/amplification process. By “complementary” is meant that the nucleotide sequence of a primer is such that the primer can form a stable hydrogen bond complex with the template; i.e., the primer can hybridize or anneal to the template by virtue of the formation of base-pairs over a length of at least ten consecutive base pairs.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the term “protein” refers to a large molecule composed of one or more chains of amino acids in a specific order. The order is determined by the base sequence of nucleotides in the gene coding for the protein. Proteins are required for the structure, function, and regulation of the body's cells, tissues, and organs. Each protein has a unique function.

As used herein, a “template” refers to a target polynucleotide strand, for example, without limitation, an unmodified naturally-occurring DNA strand, which a polymerase uses as a means of recognizing which nucleotide it should next incorporate into a growing strand to polymerize the complement of the naturally-occurring strand. Such DNA strand may be single-stranded or it may be part of a double-stranded DNA template. In applications of the present disclosure requiring repeated cycles of polymerization, e.g., the polymerase chain reaction (PCR), the template strand itself may become modified by incorporation of modified nucleotides, yet still serve as a template for a polymerase to synthesize additional polynucleotides.

A “thermocyclic reaction” is a multi-step reaction wherein at least two steps are accomplished by changing the temperature of the reaction.

A “thermostable polymerase” refers to a DNA or RNA polymerase enzyme that can withstand extremely high temperatures, such as those approaching 100° C. Often, thermostable polymerases are derived from organisms that live in extreme temperatures, such as Thermus aquaticus. Examples of thermostable polymerases include Taq, Tth, Pfu, Vent, deep vent, UlTma, and variations and derivatives thereof.

Typically, the annealing of the primers to the target DNA sequence is carried out for about 2 minutes at about 37-55° C., extension of the primer sequence by the polymerase enzyme (such as Taq polymerase) in the presence of nucleoside triphosphates is carried out for about 3 minutes at about 70-75° C., and the denaturing step to release the extended primer is carried out for about 1 minute at about 90-95° C. However, these parameters can be varied, and one of skill in the art would readily know how to adjust the temperature and time parameters of the reaction to achieve the desired results. For example, cycles may be as short as 10, 8, 6, 5, 4.5, 4, 2, 1, 0.5 minutes or less.

Also, “two temperature” techniques can be used where the annealing and extension steps may both be carried out at the same temperature, typically between about 60-65° C., thus reducing the length of each amplification cycle and resulting in a shorter assay time.

Typically, the reactions described herein are repeated until a detectable amount of product is generated. Often, such detectable amounts of product are between about 10 ng and about 100 ng, although larger quantities, e.g. 200 ng, 500 ng. I mg or more can also, of course, be detected. In terms of concentration, the amount of detectable product can be from about 0.01 pmol, 0.1 pmol, 1 pmol, 10 pmol, or more. Thus, the number of cycles of the reaction that are performed can be varied, the more cycles are performed, the more amplified product is produced. In certain embodiments, the reaction comprises 2, 5, 10, 15, 20, 30, 40, 50, or more cycles.

For example, the PCR reaction may be carried out using about 25-50 μl samples containing about 0.01 to 1.0 ng of template amplification sequence, about 10 to 100 pmol of each generic primer, about 1.5 units of Taq DNA polymerase (Promega Corp.), about 0.2 mM dDATP, about 0.2 mM dCTP, about 0.2 mM dGTP, about 0.2 mM dTTP, about 15 mM MgCl₂, about 10 mM Tris-HCl (pH 9.0), about 50 mM KCl, about 1 g/ml gelatin, and about 10 μl/ml Triton X-100 (Saiki, 1988).

Those of skill in the art are aware of the variety of nucleotides available for use in the cyclic polymerase mediated reactions. Typically, the nucleotides will consist at least in part of deoxynucleotide triphosphates (dNTPs), which are readily commercially available. Parameters for optimal use of dNTPs are also known to those of skill, and are described in the literature. In addition, a large number of nucleotide derivatives are known to those of skill and can be used in the present reaction. Such derivatives include fluorescently labeled nucleotides, allowing the detection of the product including such labeled nucleotides, as described below. Also included in this group are nucleotides that allow the sequencing of nucleic acids including such nucleotides, such as chain-terminating nucleotides, dideoxynucleotides and boronated nuclease-resistant nucleotides. Commercial kits containing the reagents most typically used for these methods of DNA sequencing are available and widely used. Other nucleotide analogs include nucleotides with bromo-, iodo-, or other modifying groups, which affect numerous properties of resulting nucleic acids including their antigenicity, their replicatability, their melting temperatures, their binding properties, etc. In addition, certain nucleotides include reactive side groups, such as sulfhydryl groups, amino groups, or N-hydroxysuccinimidyl groups, that allow the further modification of nucleic acids comprising them.

For the purposes of the present disclosure, sequence identity or homology is determined by comparing the sequences when aligned so as to maximize overlap and identity while minimizing sequence gaps. In particular, sequence identity may be determined using any of a number of mathematical algorithms. A non-limiting example of a mathematical algorithm used for comparison of two sequences is the algorithm of Karlin & Altschul, (1990) Proc. Natl. Acad Sci. USA 87: 2264-2268, modified as in Karlin & Altschul, (1993) Proc. Natl. Acad Sci. USA 90: 5873-5877.

Another example of a mathematical algorithm used for comparison of sequences is the algorithm of Myers & Miller. CABIOS 1988; 4: 11-17. Such an algorithm is incorporated into the ALIGN program (version 2.0) which is part of the GCG sequence alignment software package. When utilizing the ALIGN program for comparing amino acid sequences, a PAM 120 weight residue table, a gap length penalty of 12, and a gap penalty of 4 can be used. Yet another useful algorithm for identifying regions of local sequence similarity and alignment is the FASTA algorithm as described in Pearson & Lipman, (1988) Proc. Natl. Acad Sci. USA 85: 2444-2448.

Advantageous for use according to the present disclosure is the WU-BLAST (Washington University BLAST) version 2.0 software. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., (1990) J. Mol. Biol 215: 403-410; Gish & States (1993); Nature Genetics 3: 266-272; Karlin & Altschul, (1993) Proc. Natl. Acad. Sci. USA 90: 5873-5877; all of which are incorporated by reference herein).

In all search programs in the suite the gapped alignment routines are integral to the database search itself. Gapping can be turned off if desired. The default penalty (Q) for a gap of length one is Q-9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Alternatively or additionally, the term “homology” or “identity”, for instance, with respect to a nucleotide or amino acid sequence, can indicate a quantitative measure of homology between two sequences. The percent sequence homology can be calculated as (N_{ref}−N_{dif})*₁₀₀/N_{ref}, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N_{ref}=8; N_{dif}=2). “Homology” or “identity” can refer to the number of positions with identical nucleotides or amino acids divided by the number of nucleotides or amino acids in the shorter of the two sequences wherein alignment of the two sequences can be determined in accordance with the Wilbur and Lipman algorithm (Wilbur & Lipman, (1983) Proc. Natl. Acad. Sci. U.S.A. 80: 726, incorporated herein by reference), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite. Intelligenetics Inc. CA). When RNA sequences are said to be similar, or have a degree of sequence identity or homology with DNA sequences, thymidine (T) in the DNA sequence is considered equal to uracil (U) in the RNA sequence. Thus, RNA sequences are within the scope of the disclosure and can be derived from DNA sequences, by thymidine (T) in the DNA sequence being considered equal to uracil (U) in RNA sequences. Without undue experimentation, the skilled artisan can consult with many other programs or references for determining percent homology.

Further definitions are provided in context below. Unless otherwise defined, all technical and scientific terms used herein have the same meaning as commonly understood by one of ordinary skill in the art of molecular biology. Although methods and materials similar or equivalent to those described herein can be used in the practice or testing of the present disclosure, suitable methods and materials are described herein.

The primers and probes described herein may be readily prepared by, for example, directly synthesizing the fragment by chemical means or by introducing selected sequences into recombinant vectors for recombinant production. Methods for making a vector or recombinants or plasmid for amplification of the fragment either in vivo or in vitro can be any desired method, e.g., a method which is by or analogous to the methods disclosed in, or disclosed in documents cited in: U.S. Pat. Nos. 4,603,112; 4,769,330; 4,394,448; 4,722,848; 4,745,051; 4,769,331; 4,945,050; 5,494,807; 5,514,375; 5,744,140; 5,744,141; 5,756,103; 5,762,938; 5,766,599; 5,990,091; 5,174,993; 5,505,941; 5,338,683; 5,494,807; 5,591,639; 5,589,466; 5,677,178; 5,591,439; 5,552,143; 5,580,859; 6,130,066; 6,004,777, 6,130,066; 6,497,883; 6,464,984; 6,451,770; 6,391,314; 6,387,376; 6,376,473; 6,368,603; 6,348,196; 6,306,400; 6,228,846; 6,221,362; 6,217,883; 6,207,166; 6,207,165; 6,159,477; 6,153,199; 6,090,393; 6,074,649; 6,045,803; 6,033,670; 6,485,729; 6,103,526; 6,224,882; 6,312,682; 6,348,450 and 6; 312,683; U.S. patent application Ser. No. 920,197, filed Oct. 16, 1986; WO 90/01543; W091/11525; WO 94/16716; WO 96/39491; WO 98/33510; EP 265785; EP 0 370 573; Andreansky et al., Proc. Natl. Acad Sci. U.S.A. 1996; 93:11313-11318; Ballay et al., EMBO J. 1993; 4:3861-65; Felgner et al., J. Biol. Chem. 1994; 269:2550-2561; Frolov et al., Proc. Natl. Acad. Sci. USA 1996; 93:11371-11377; Graham, Tibtech 1990; 8:85-87; Grunhaus et al., Sem. Virol. 1992; 3:237-52; Ju et al., Diabetologia 1998; 41:736-739; Kitson et al., J. Virol. 1991; 65:3068-3075; McClements et al., Proc. Natl. Acad. Sci. USA 1996; 93:11414-11420; Moss. Proc. Natl. Acad. Sci. USA 1996; 93:11341-11348; Paoletti, Proc. Natl. Acad. Sci. USA 1996; 93:11349-11353; Pennock et al., Mol. Cell. Biol. 1984; 4:399-406; Richardson (Ed). Methods in Molecular Biology 1995; 39, “Baculovirus Expression Protocols,” Humana Press Inc.; Smith et al. (1983) Mol. Cell. Biol. 1983; 3:2156-2165; Robertson et al., Proc. Natl. Acad. Sci. USA 1996; 93:11334-11340; Robinson et al., Sem. Immunol. 1997; 9:271; and Roizman, Proc. Natl. Acad. Sci. USA 1996; 93:11307-11312.

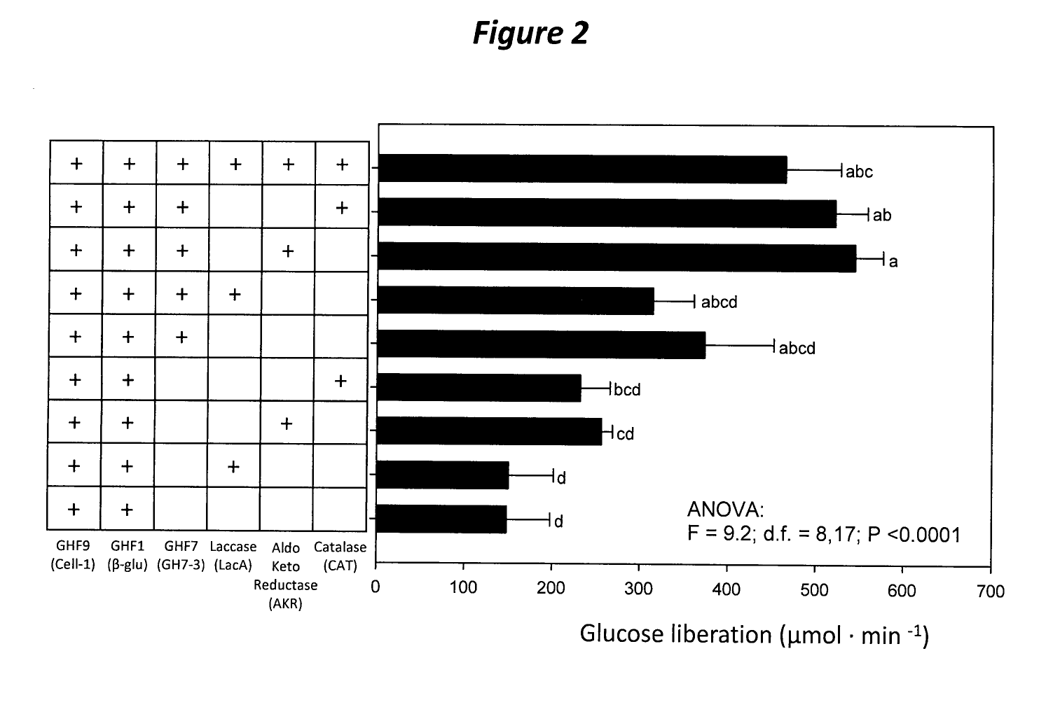

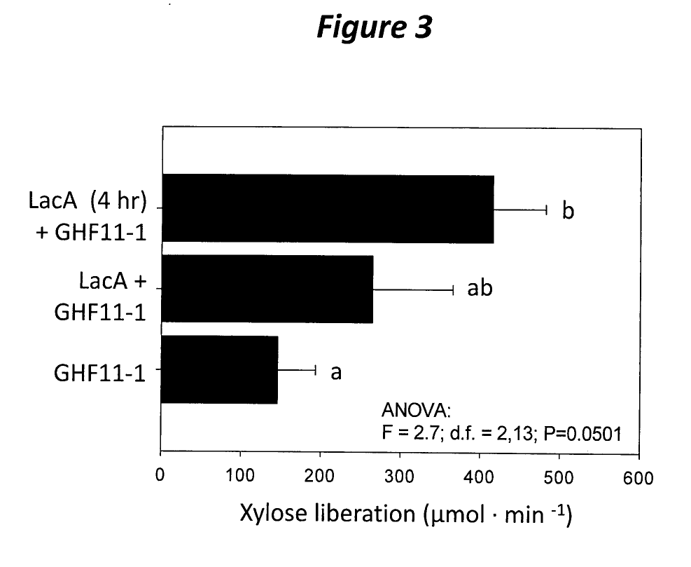

The present disclosure encompasses systems and methods of use of said systems for the generation of fermentable compounds from lignified plant material using enzymes, or catalytically active domains thereof, derived from the gut of termites, whether encoded by the termite genome or by that of a symbiont organism. The systems comprises at least two termite-derived enzymes or the catalytically active domains thereof, that can cooperate to degrade lignified plant material to a fermentable compound such as, but not limited to, glucose, xylose, and the like. In particular, but not limiting, a combination of Cell-1, β-glucosidase, and GHF7 and a catalase release significant levels of glucose from lignified plant material. It is however, contemplated to be within the scope of the disclosure for other combinations of enzyme activities to be formed based on the core pairing of the Cell-1, β-glucosidase, as shown in FIGS. 2 and 3.

While enzymes or active fragments thereof may be isolated from tissues of termites, it is contemplated that nucleotide sequences encoding such polypeptides may be inserted into suitable expression vectors for the expression of the proteins in an in vitro system such as cultured cells. The enzymes or derivatives of such may then be isolated by methods well known in the art and combined with plant material under conditions allowing the enzymes to catalyze the breakdown of the lignin, cellulose, or hemicellulose, into small sugar moieties.

The vast majority of termite digestive research has focused on cellulose digestion. However, the present disclosure provides an integrative approach to specifically resolve the question of how termites cope with their lignin-rich lignocellulose diets. Using a selective feeding approach and diets containing differing degrees of lignin complexity, over 9,000 differentially expressed host and symbiont transcripts that include over 300 responsive lignase/antioxidant, cellulase and hemicellulase transcripts were sequenced. Using protein-based approaches, congruence between our transcription and translation-level results was shown. The complex enzymatic machinery termites use to digest dietary lignocellulose was shown and support the idea that lignin and its degradation products present termites with significant xenobiotic challenges. Clearly, these challenges must be effectively overcome for termites and their gut symbiota to survive.

In addition to a previously identified LacA protein, embodiments of the present disclosure provide two candidate lignase/phenoloxidase enzyme families not previously considered in connection with lignocellulose saccharification: AKR and CAT. Recombinant AKR, CAT and LacA proteins, which apparently play no roles in cellulose and hemicellulose metabolism, have transcripts that are inducible by lignin feeding. Each significantly enhances lignocellulose saccharification by host and symbiont cellulases and/or xylanases. Thus, the present disclosure provides several important new enzyme families useful in the production of biofuels and other biomass-based goods.

The present disclosure encompasses systems of isolated enzymes derived from a termite or symbionts of a termite that are able to degrade the molecular structures of the components of lignified plant material to provide fermentable compounds, particularly sugars, that are useful for the production of biofuels. The methods of the disclosure allow the isolation of nucleic acid sequences encoding enzymes, or fragments thereof, that are used by termites or symbionts resident in the gut of termites and which are associated with the breakdown in vivo of ingested plant material to provide nutrients and energy sources for the insect. While it is possible to obtain polypeptides that encompass the entire amino acid sequences of the enzymes of the systems herein disclosed, it is further contemplated that truncated variants of the polypeptides may be produced by suitably locating PCR amplification primers such that the fore-shortened polypeptides may retain the catalytic activity of the native enzymes.

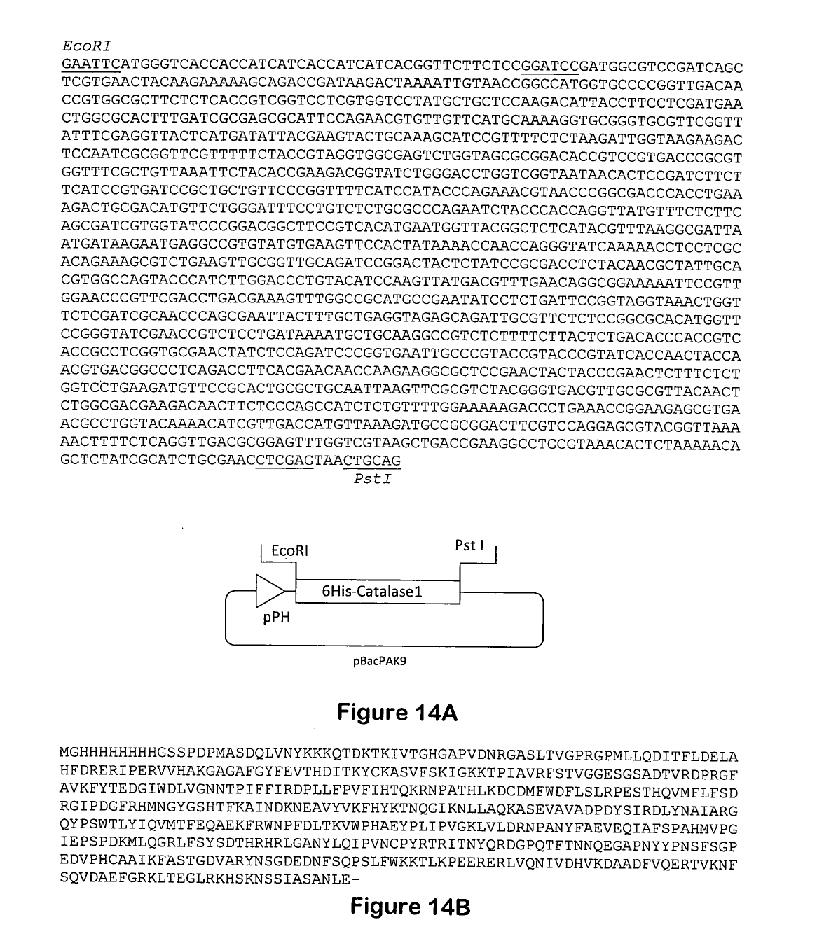

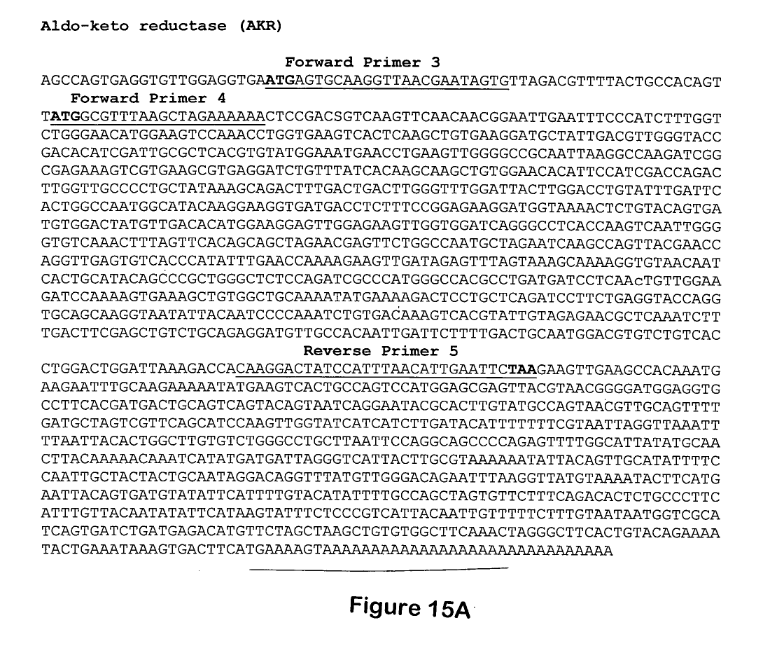

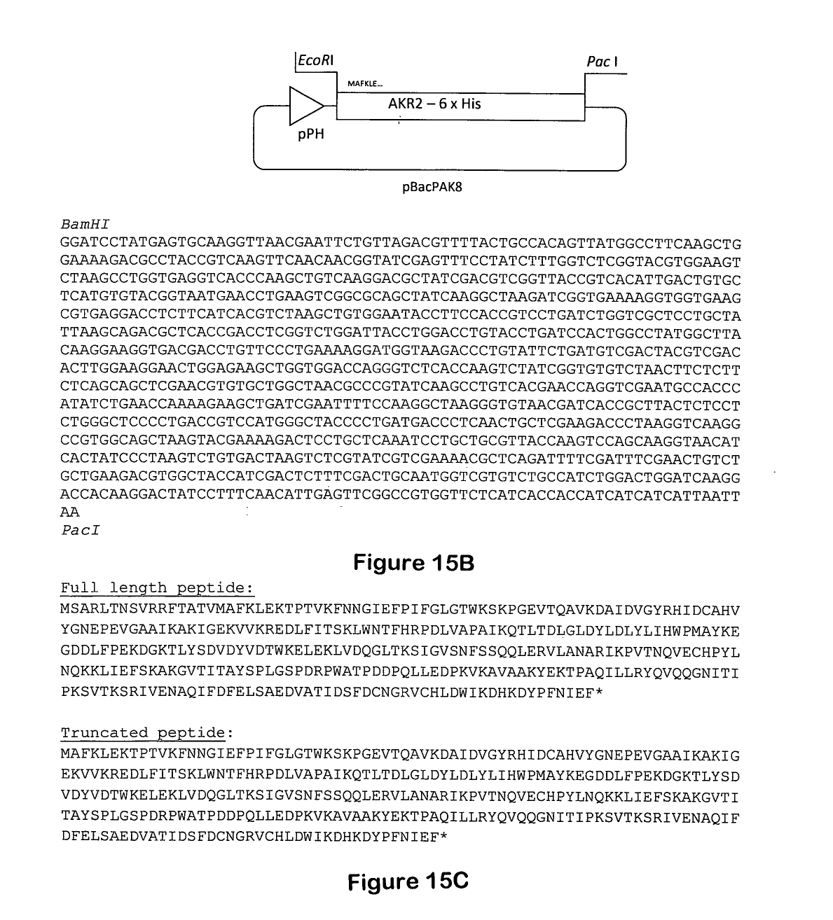

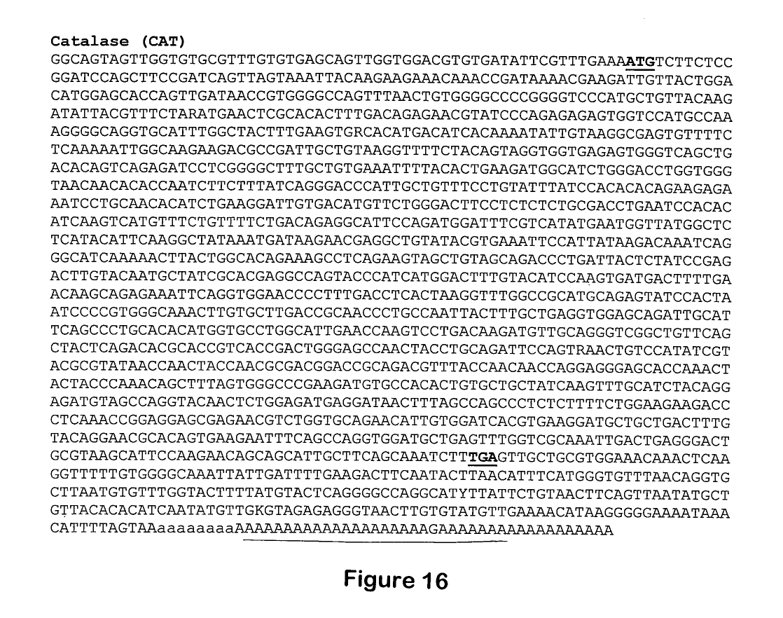

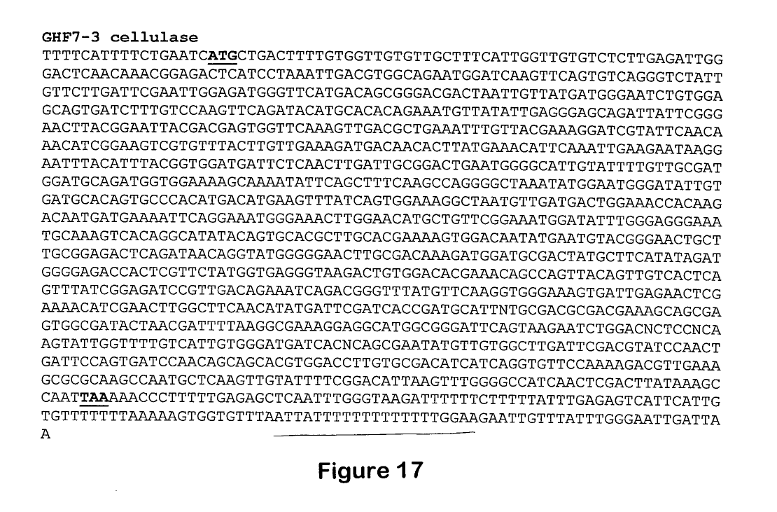

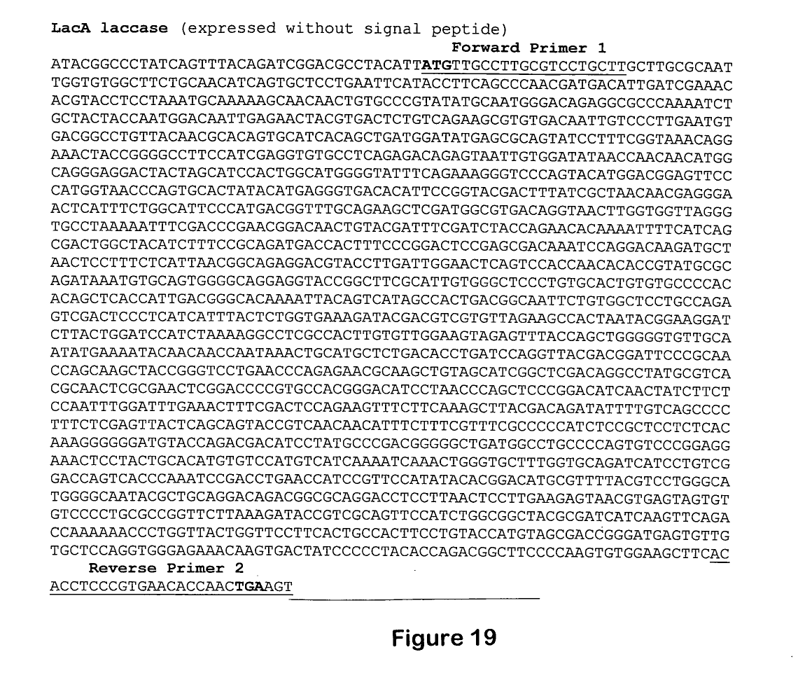

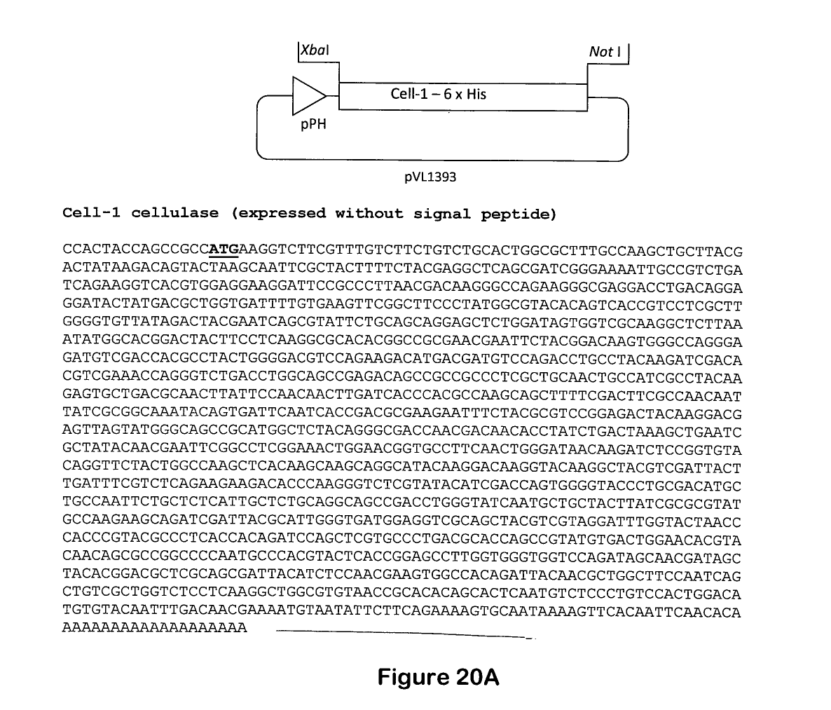

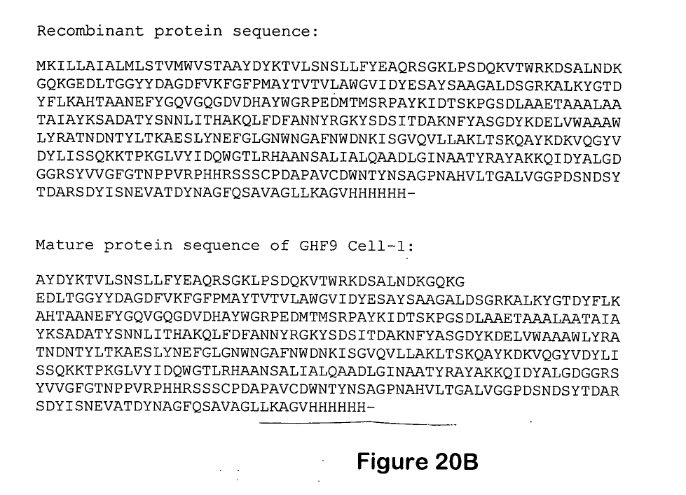

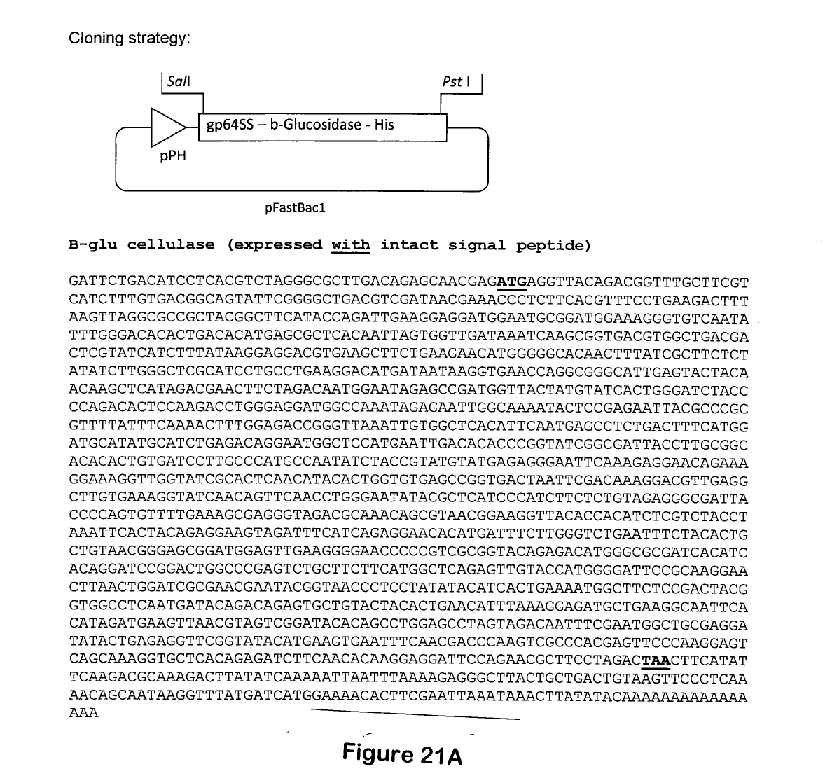

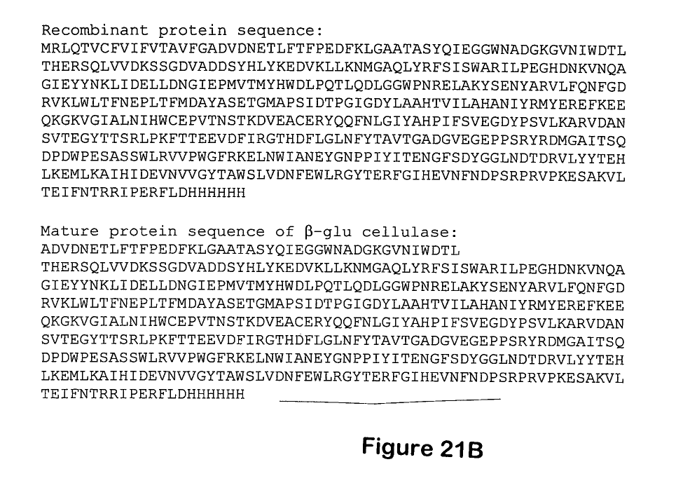

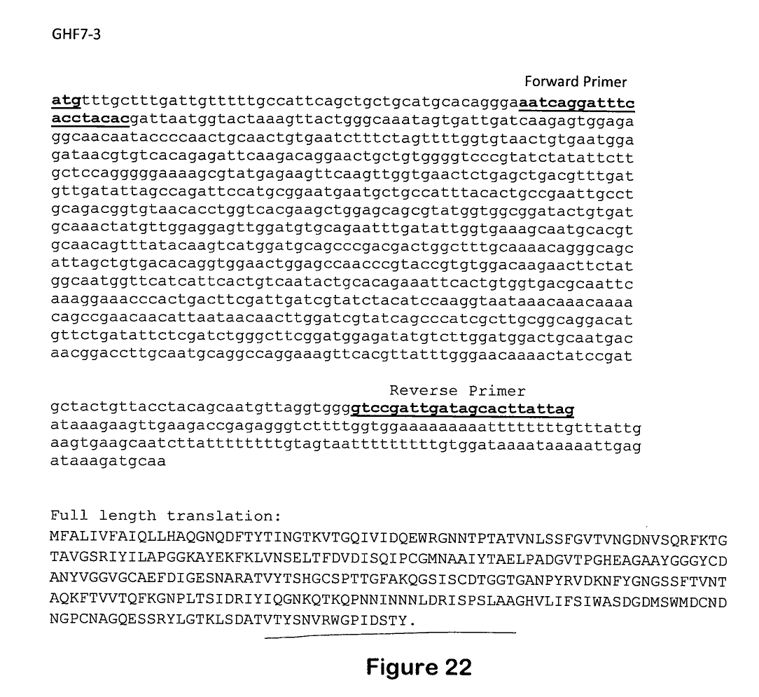

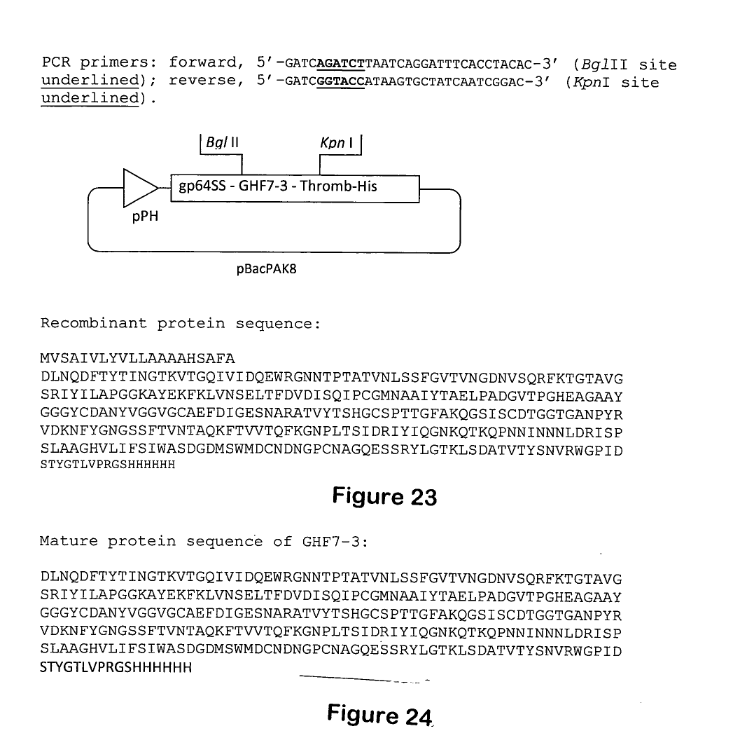

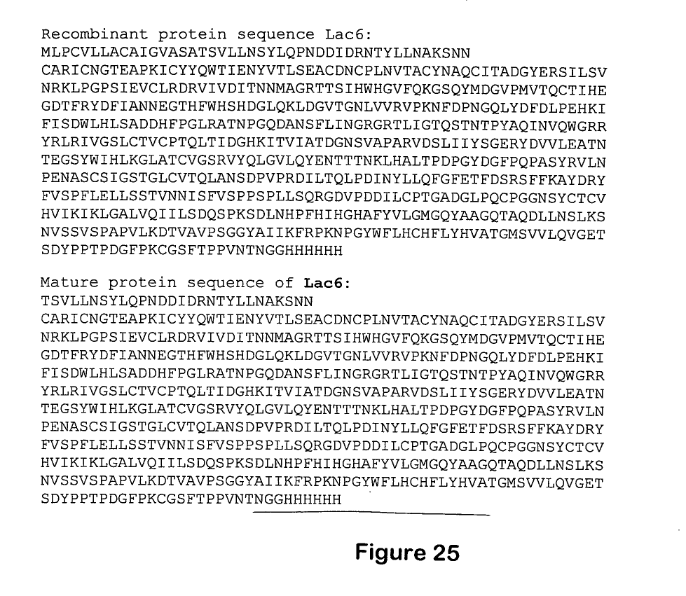

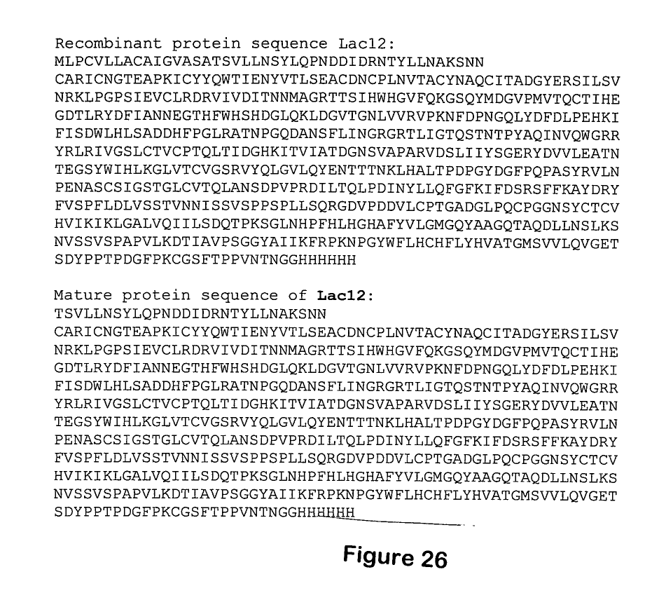

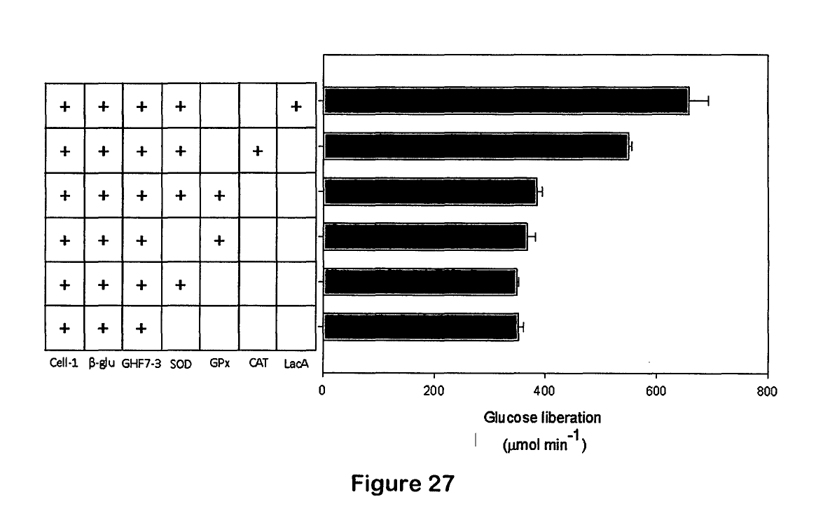

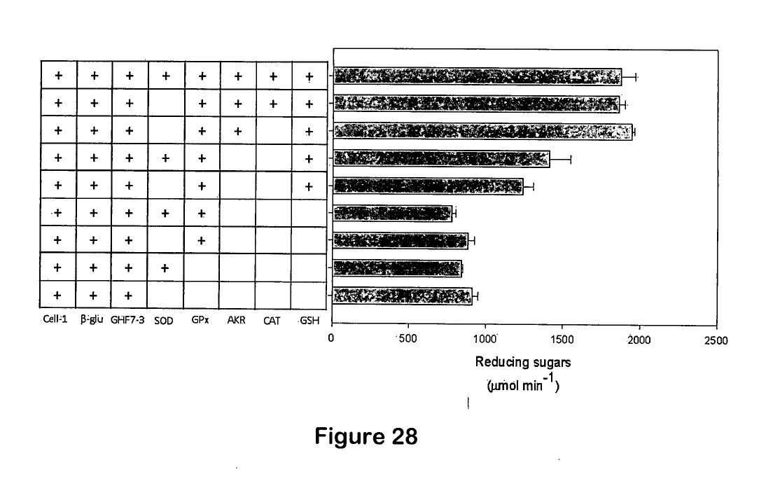

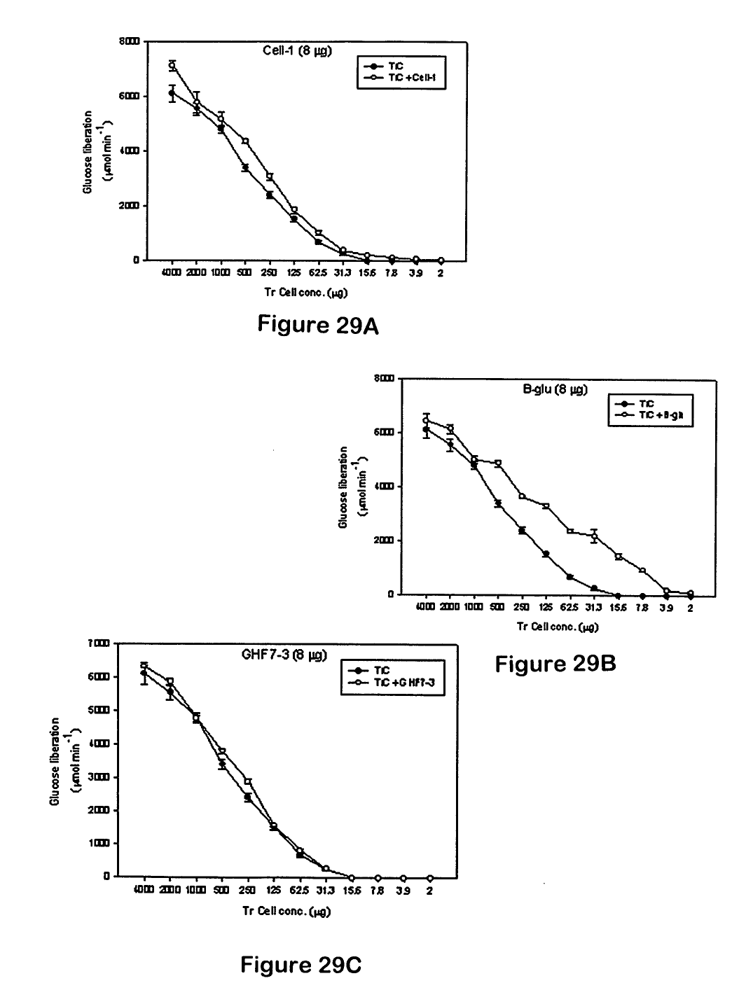

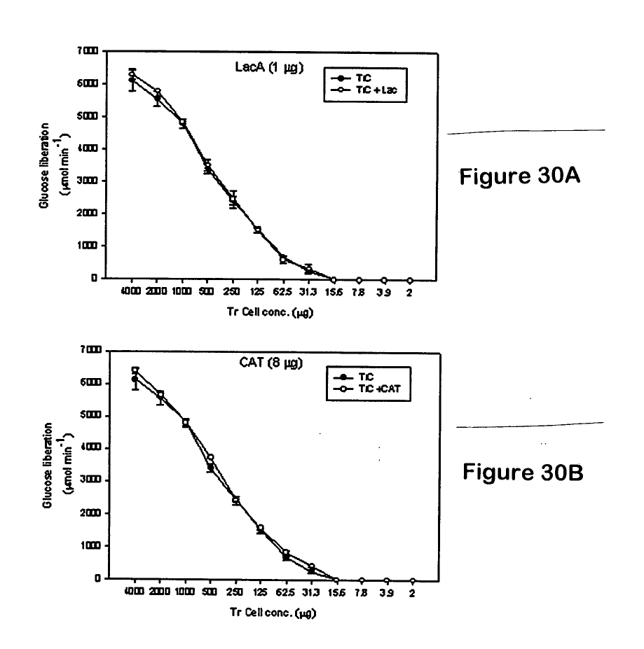

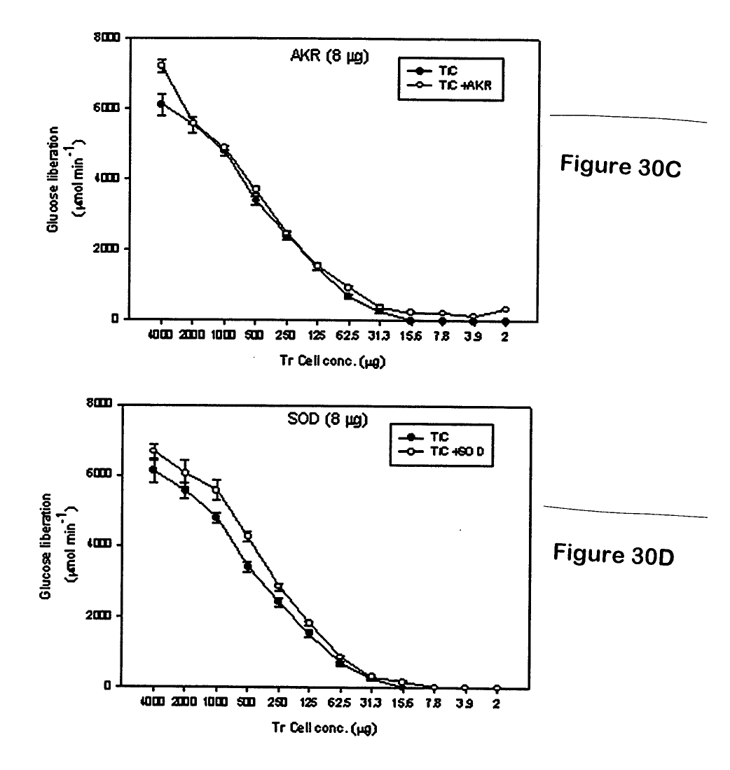

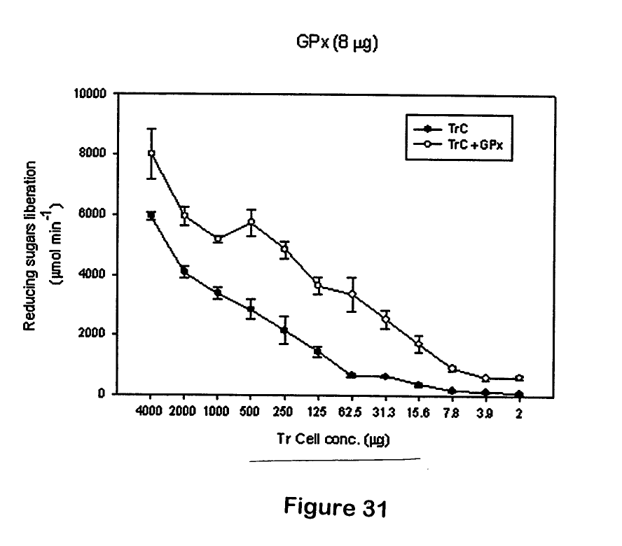

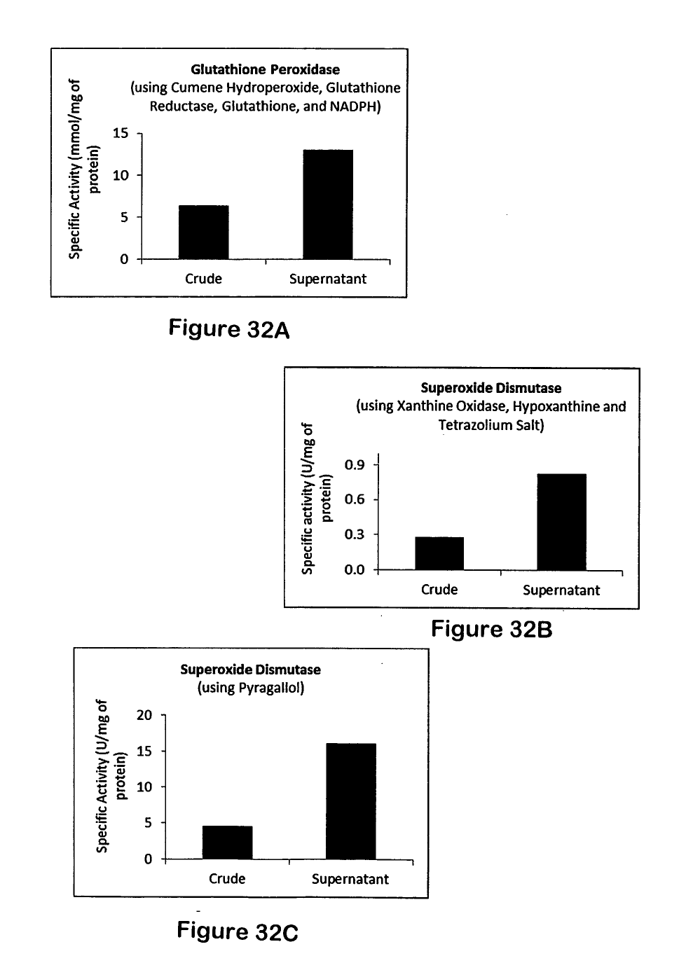

The enzymes identified by the methods of the disclosure may be provided as expressed products from in vitro or heterologous expression systems that allow for the isolation of the expressed products and their substantial purification. Thereafter, the isolated enzymatically active polypeptides of the disclosure may be combined in vitro to provide systems suitable for the digestion of plant material into fermentable products. The coding sequences of the recombinant enzymes are provided in FIGS. 14-26. The skilled artisan, being in possession of said sequences and the examples of this disclosure, would be able to recombinantly express said enzymes without additional experimentation.

In an alternative embodiment, the enzymes of the present claimed system may be partially isolated from termite guts, wherein the termites have been fed the appropriate lignin- or cellulose-rich diet to induce the overexpression in vivo of the enzymes of the present invention.

While not intended to be limiting, examples of termite encoded enzymatically active polypeptides include Cell-1, β-glu, cellulase GHF7-3, an aldo-keto-reductase, a catalase, and a laccase. The isolated polypeptides (recombinant intact or variant forms thereof) may be combined in a range of systems, including, but not limited to, (i) Cell-1, β-glu, cellulase GHF7-3, LacA, aldo-keto-reductase, and a catalase; (ii) Cell-1, β-glu, cellulase GHF7-3, and a catalase; (iii) Cell-1, β-glu, cellulase GHF7-3, and aldo-keto-reductase; (iv) Cell-1, β-glu, cellulase GHF7-3, and LacA; (v) Cell-1, β-glu, and cellulase GHF7-3; (vi) Cell-1, β-glu, and a catalase; (vii) Cell-1, β-glu, and aldo-keto-reductase; or (viii) LacA AND GHF11-1.

For example, in one embodiment of the systems of the disclosure, PCR primers 3 and 5 may be used to PCR amplify the entire aldo-keto reductase (AKR) polypeptide or, by using the primers 4 and 5, an N-terminus truncated variant thereof. It has been determined that each variant so generated and expressed in a suitable expression system, a baculovirus vector-lepidopteran larva host, exhibits similar enzymatic activity with a suitable substrate, and either variant may be included in the systems of the present disclosure for the generation of fermentable products from plant material.

Also identified are the enzymes “SOD” and “GPX.” The enzymes (1) target lignin, which presents a substantial barrier to ethanol production from 2nd generation (non-food) feedstocks, and/or (2) synergize release of fermentable monosaccharides from lignocellulosic biomass through an as-yet undetermined mechanism. The method and arrangement includes isolating the termite digestive enzymes and applying the isolated enzymes to biomass for conversion of the biomass. These enzymes can be used to make fermentable sugars more accessible when producing bioethanol from plant-based lignocellulose feedstocks.

One aspect of the disclosure, therefore, encompasses embodiments of a system for producing a fermentable product from a lignified plant material, the system comprising either: (i) the catalytically active domains, or polypeptides comprising said catalytically active domains, of cellulases Cell-1 and □-glu, and further comprising a catalytically active domain of at least one enzyme selected from the group consisting of: a cellulase, an aldo-keto-reductase, a catalase, and a laccase; or (ii) the catalytically active domains, or polypeptides comprising said catalytically active domains, of an endo-xylanase and, optionally, a laccase, where the catalytically active domains, or polypeptides comprising said catalytically active domains, can cooperate to provide a fermentable product from a lignified plant material.

In embodiments according to this aspect of the disclosure, at least one of Cell-1, β-glu, cellulase, an aldo-keto-reductase, a catalase, a laccase, and a endo-xylanase is derived from a termite or symbiont thereof.

In embodiments according to this aspect of the disclosure, at least one of Cell-1, β-glu, cellulase, an aldo-keto-reductase, a catalase, a laccase, and a endo-xylanase is expressed from a recombinant nucleotide sequence.

In embodiments according to this aspect of the disclosure, the system can comprise a series of isolated recombinant polypeptides, where each polypeptide comprises a catalytically active domain and is expressed from an expression vector of a recombinant expression system, and the recombinant expression system can be selected from a eukaryotic cell-based system and a prokaryotic cell-based system.

In embodiments of the system according to this aspect of the disclosure, the expression vector can be a baculovirus expression vector and the recombinant expression system is a eukaryotic cell-based system.

In embodiments of the system according to this aspect of the disclosure, the system can be at least one of the Cell-1, the 1-glu, the cellulase, the aldo-keto-reductase, the catalase, the laccase, and the endo-xylanase is derived from a termite.

In embodiments of the system according to this aspect of the disclosure, the catalytically active domains, or polypeptides comprising said catalytically active domains, can cooperate to provide a sugar from a lignified plant material.

In embodiments of the system according to this aspect of the disclosure, the catalytically active domains can cooperate to provide glucose from a lignified plant material.

In embodiments of the system according to this aspect of the disclosure, the catalytically active domains, or polypeptides comprising said catalytically active domains, can cooperate to provide a pentose from a lignified plant material.

In embodiments of the system according to this aspect of the disclosure, catalytically active domains, or polypeptides comprising said catalytically active domains can be Cell-1 and β-glu, and at least one of the group consisting of: cellulase GHF7-3, an aldo-keto-reductase, a catalase, and a laccase.

In embodiments of the system according to this aspect of the disclosure, The system can comprise the catalytically active domains, or polypeptides comprising said catalytically active domains, of Cell-1, β-glu, and cellulase GHF7-3, and either an aldo-keto-reductase or a catalase.

In embodiments of the system according to this aspect of the disclosure, the system can comprise the catalytically active domains, or polypeptides comprising said catalytically active domains, of an endo-xylanase and a laccase.

In embodiments of the system according to this aspect of the disclosure, the laccase can be LacA.

In embodiments of the system according to this aspect of the disclosure, the system can comprise the catalytically active domains, or polypeptides comprising said catalytically active domains, of: (i) Cell-1, β-glu, cellulase GHF7-3, LacA, aldo-keto-reductase, and a catalase; (ii) Cell-1, β-glu, cellulase GHF7-3, and a catalase; (iii) Cell-1, β-glu, cellulase GHF7-3, and aldo-keto-reductase; (iv) Cell-1, β-glu, cellulase GHF7-3, and LacA; (v) Cell-1, β-glu, and cellulase GHF7-3; (vi) Cell-1, β-glu, and a catalase; (vii) Cell-1, β-glu, and aldo-keto-reductase, or (viii) LacA AND GHF11-1.

Another aspect of the disclosure encompasses embodiments of an expression vector encoding a polypeptide, or a catalytically active variant thereof, selected from the group consisting of: Cell-1, β-glu, cellulase GHF7-3, LacA, aldo-keto-reductase, a catalase, and GHF11-1

In embodiments of this aspect of the disclosure the expression vector can be in a host cell or tissue.

In some embodiments of this aspect of the disclosure, the expression vector can be derived from a baculovirus.

Another aspect of the disclosure encompasses embodiments of a method of converting a lignified plant material to a fermentable product, the method comprising the steps of: (a) obtaining a system of catalytically active domains, or polypeptides comprising said catalytically active domains, according to any of the above paragraphs; and (b) incubating the system with a source of lignified plant material under conditions allowing the polypeptides to cooperatively produce a fermentable product from the lignified plant material.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and protected by the following embodiments.

The following examples are put forth so as to provide those of ordinary skill in the art with a complete disclosure and description of how to perform the methods and use the compositions and compounds disclosed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified.

### EXAMPLES

### Example 1

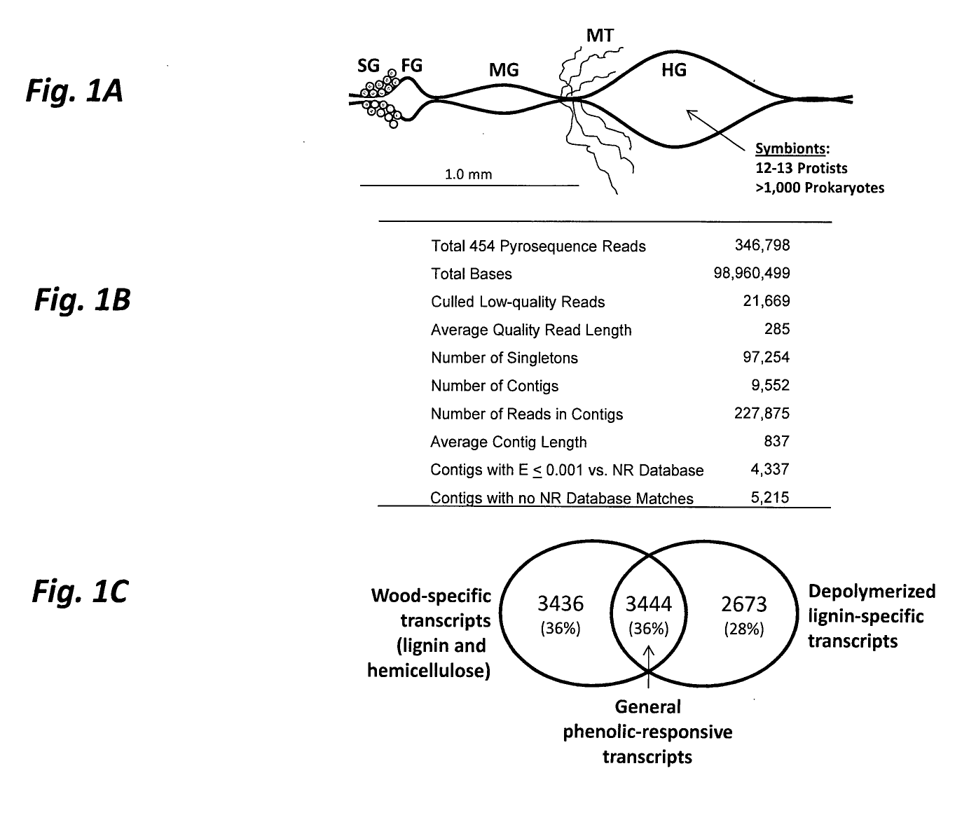

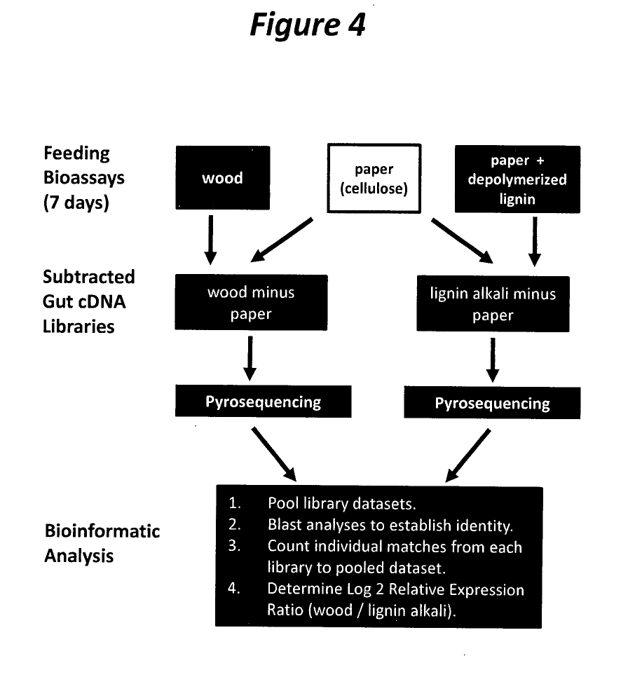

Groups of R. flavipes workers (200 per treatment; 50 from each of four colonies) received three diet treatments for 7 days before isolation of total gut RNA (FIG. 4). Diet treatments included highly pure cellulose (filter paper), complex lignocellulose (pine wood), and cellulose+depolymerized lignin (i.e., lignin alkali), which is an industrial de-lignification byproduct containing depolymerized lignin and related phenolic compounds. The isolation of whole-gut RNA after 7d enabled digestome-wide sampling for expressed transcripts from host termite gut tissue and eukaryotic protist gut symbionts (FIG. 1A). One mRNA pool was isolated for each feeding treatment. To enrich for lignin- and other phenolic-responsive transcripts, the cellulose-fed mRNA pool was subtracted from the wood and depolymerized lignin preparations to create two “subtracted” cDNA libraries. The two subtracted libraries were subjected to 454 titanium pyrosequencing using established parameters and the contig sequences were assembled de novo under genome settings.

Sequencing Overview:

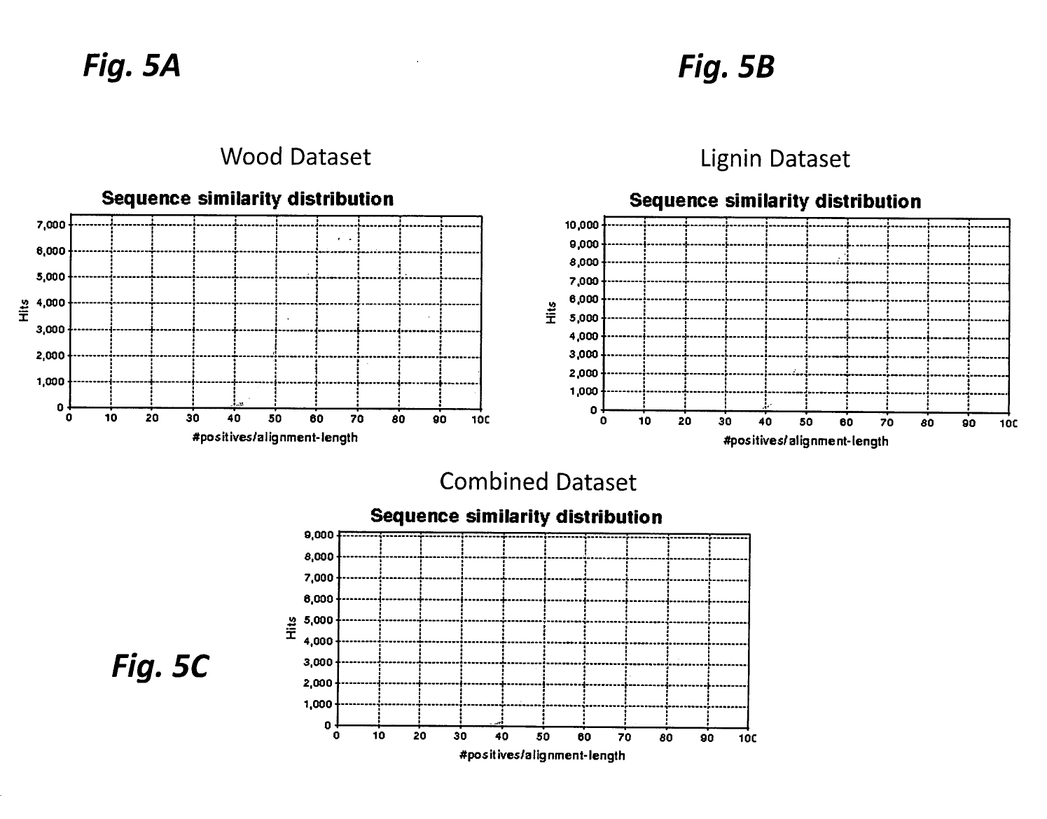

From the two subtracted libraries, 346,798 sequencing reads were obtained that provided 98,960,499) nucleotide bases with an average read length of 285 nucleotide bases (FIG. 1B). Resulting sequences were assembled into 9,552 multiple read contigs and 97,254 single read singletons. Of the 9,552 differentially expressed contigs, 3,444 were uniformly represented in the two cellulose-subtracted libraries, and thus were considered to be phenolic-responsive (FIG. 1C). A total of 3,436 contigs were unique to the wood library and 2,763 to the depolymerized lignin library. The wood-library transcripts are considered to be responsive to intact/polymerized lignin and hemicellulose; whereas, the depolymerized lignin-library transcripts are considered to be responsive to depolymerized lignin and related degradation products. Sequence similarity length assessments showed a normal distribution with median values of 60-75 bases (FIGS. 5A-5B).

### Example 2

### BLAST Summaries

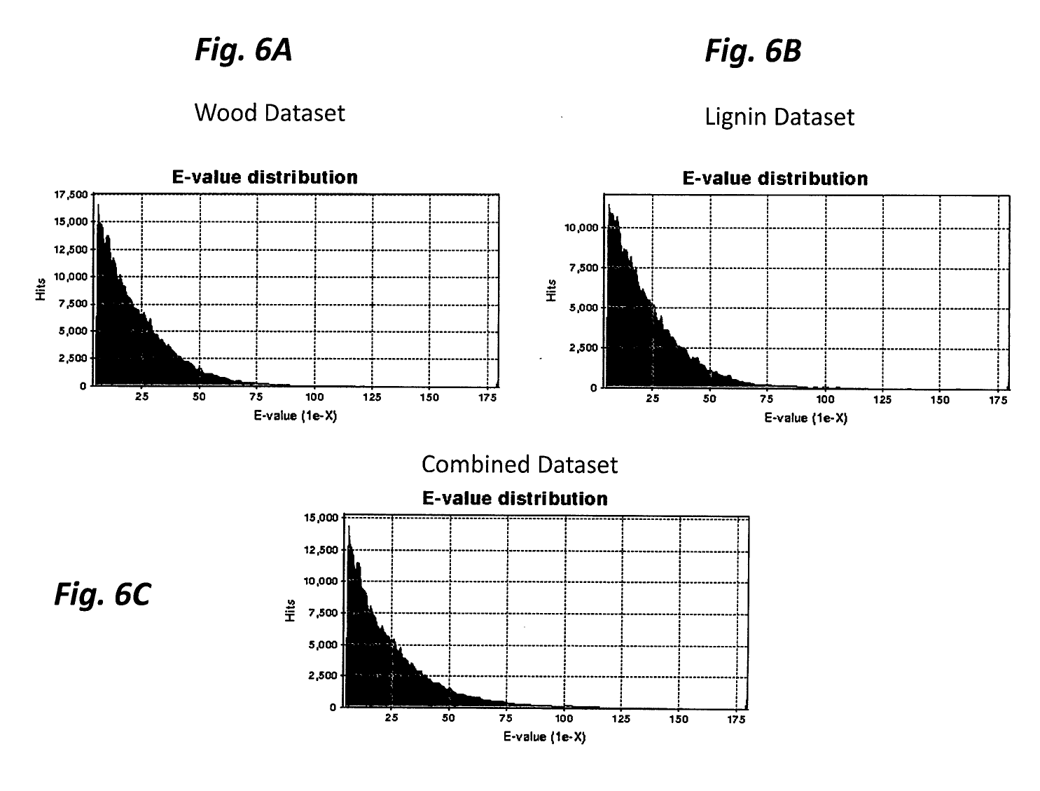

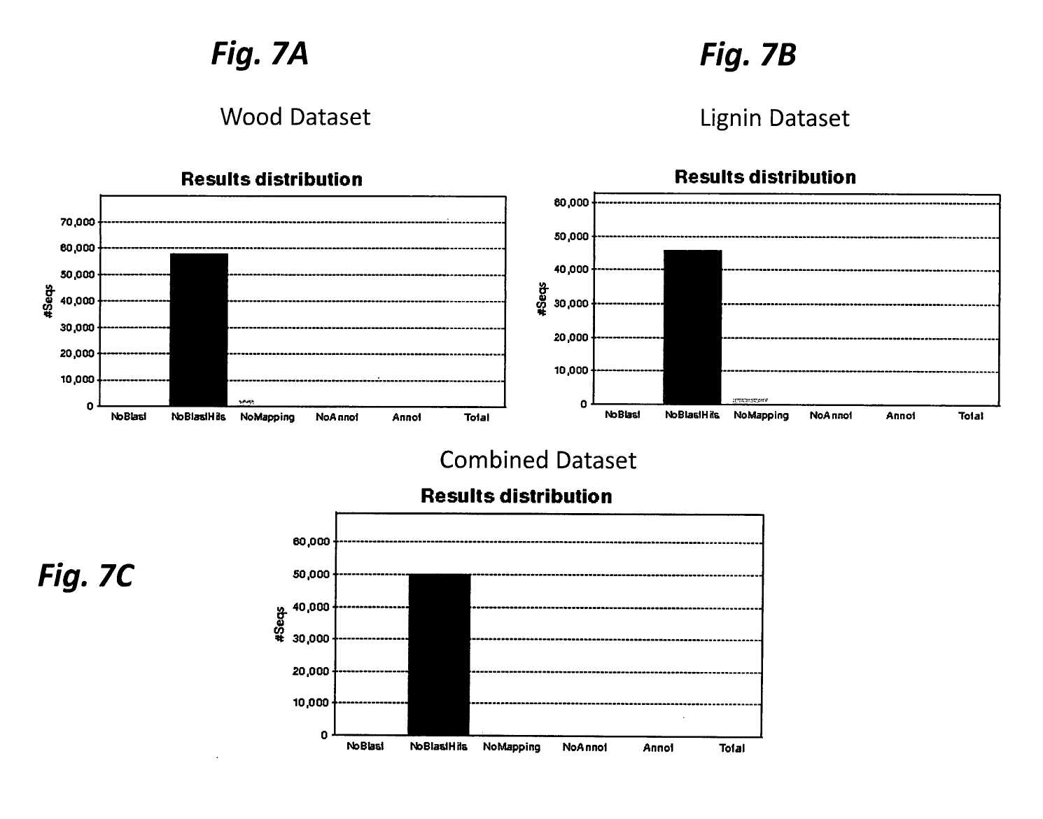

Consistent with a previous R. flavipes sequencing project (Tartar et al., Biotechnol. Biofuels 2, 25 (2009), e-value distributions for sequence database matches indicated most individual sequence reads had no translated BLASTx database matches (FIGS. 6A-6C and 7A-7C). However, the number of translated (BLASTx) database matches improved when using contigs generated from the combined wood and lignin libraries. i.e., 45% of the combined dataset contigs (n=4,337) had significant matches. Genome database matches from the pyrosequence dataset included multiple insect genomes, as well as the genome of the protist Trichomonas vaginalis and a number of insect symbiont genomes and metagenomes. Comparatively fewer prokaryote and euryarchaeota sequences were identified. These taxonomic characteristics reflect the eukaryotic poly-A RNA targeted approach, as well as the unique eukaryotic host-symbiont relationship that exists in R. flavipes.

### Example 3

### Gene Ontology (GO) Summaries

A total of 37,243 Gene Ontology (GO) terms were assigned to the combined wood and lignin feeding dataset based on BLAST matches to sequences with known function.

GO results for the top 100 most expressed transcripts overall were compared to the top 100 most expressed transcripts from the wood and depolymerized lignin libraries in the three GO categories: cellular location, molecular function and biological process. There were fewer GO classifications represented in the wood and lignin libraries resulting from all three database searches, suggesting the wood and depolymerized lignin-fed transcript pools are less diverse and more specialized than the general sequence pool. With respect to cellular location, both libraries were enriched in protein complex and membrane-associated GO categories, particularly the wood library, in which nearly half of annotations were membrane associated. This result is consistent with the membrane-bound nature of insect xenobiotic detoxification systems and the idea that lignin and other compounds present in wood are toxic to termites and their gut microbiota (Breznak & Brune 1994; Scharf, M. E. & Tartar. A. Biofuels Bioprod. Bioref. 2, 540 (2008)).

Molecular function GO searches revealed enriched binding and catalytic capabilities in association with wood and depolymerized lignin feeding. For example, the wood library was enriched for glycosyl hydrolases, which is consistent with the carbohydrate content of wood. Also, the depolymerized lignin library was enriched in oxidoreductase activity, which is consistent with lignin degradation and xenobiotic metabolism requiring oxygen and associated redox systems.

Biological process was the most diverse GO category overall, but again the wood and depolymerized lignin libraries were less diverse than the general “top 100” sequence pool, implying specialization. Most noteworthy of biological process GO searches were phagocytosis related transcript expression in the wood library and glycolysis related transcript expression in the lignin library. Phagocytosis is a protist symbiont-associated function that is well documented in association with wood feeding (Cleveland, L. R. (1923) Proc. Natl. Acad Sci. USA 9; 424). Lignin metabolism would likely require more initial energy input and this is suggested by increased synthesis of glycolysis related proteins such as glyceraldehyde-3-phosphate dehydrogenase and enolase. There was also increased production of arginine kinase which is important for energy metabolism in insects (Newsholme, E. A. et al. Chem. Biol. Interact. 130-132, 685 (2001)).

### Example 4

### Target Genes (Candidate Lignases and Glycosyl Hydrolases)

In agreement with GO annotations above and predictions resulting from a previous gut digestome EST project (Tartar et al. 2009), 262 relevant lignocellulase transcripts were differentially enriched in the two subtracted libraries, 96 lignase/detox enzymes and 166 carbohydrate active proteins, as shown in Table 1.

TABLE 1
Summary of differentially expressed transcripts from
the lignase/detoxification (n = 96; TOP)
and carbohydrate-active (n = 166; BOTTOM) functional
categories. Numbers indicate the number of
transcript contigs occurring in the wood-fed and
lignin-led libraries, or shared among both libraries
(i.e., “general phenolic responsive”).

| | | | | Depoly- | General | |
|---|---|---|---|---|---|---|
| | | | Wood | merized | phenolic | |
| Functional | | Gene | induc- | lignin | respon- | TO- |
| categories | | family | ible | inducible | sive | TALS |
| Candidate lignase, | | P450 | 7 | 8 | 13 | 28 |
| antioxidant | | ADH | 3 | 2 | 14 | 19 |
| and detoxification | | AKR | 5 | 4 | 8 | 17 |
| enzymes | | EST | 5 | 5 | 5 | 15 |
| | | GST | 2 | 2 | 6 | 10 |
| | | GSP | 1 | 4 | 2 | 7 |
| | | SOD | | 1 | 2 | 3 |
| | | CAT | 1 | 1 | | 2 |
| | | LAC | 1 | | 1 | 2 |
| | | | total: 24 | total: 25 | total: 47 | 96 |
| Carbo- | Cellulose | GHF 7 | 24 | 15 | 5 | 44 |
| hydrate | | GHF | 7 | | 4 | 11 |
| active | | 45 | | | | |
| | | GHF 1 | 2 | 1 | 3 | 6 |
| | | GHF 9 | | | 4 | 4 |
| | | GHF 2 | 3 | | | 3 |
| | Hemicellulase | GHF | 10 | 4 | 3 | 17 |
| | | 11 | | | | |
| | | GHF 3 | 10 | 3 | | 13 |
| | | GHF 5 | 9 | | | 9 |
| | | GHF | 5 | | 1 | 6 |
| | | 26 | | | | |
| | | GHF | 1 | 2 | 2 | 5 |
| | | 43 | | | | |
| | | GHF | 1 | 1 | 1 | 3 |
| | | 28 | | | | |
| | | GHF | 1 | 1 | | 2 |
| | | 16 | | | | |
| | | GHF | 1 | 1 | | 2 |
| | | 27 | | | | |
| | | GHF | 1 | | | 1 |
| | | 53 | | | | |
| | | GHF | | 1 | | 1 |
| | | 38 | | | | |
| | | GHF | 1 | | | 1 |
| | | 30 | | | | |
| | | GHF | 1 | | | 1 |
| | | 10 | | | | |
| | Chitinase | GHF 8 | 2 | 1 | | 3 |
| | | GHF | 1 | | | 1 |
| | | 18 | | | | |
| | Ceramidase | GHF | | 1 | | 1 |
| | | 30 | | | | |
| | Pectinase | PL1 | 5 | | 1 | 6 |
| | Amylase | GHF13 | | | 2 | 2 |
| | Laminarinase | GHF17 | | | 3 | 3 |
| | Carbohydrate | CBM2 | 4 | 1 | 1 | 6 |
| | Binding | CBM1 | 1 | | 1 | 2 |
| | | EF | 3 | | 2 | 5 |
| | | Hand | | | | |
| | | Lectin | 1 | 5 | 2 | 8 |
| | | | total: 94 | total: 37 | total: 35 | 166 |

Abbreviations: P450, cytochrome P450;
AKR, aldo-keto reductase;
EST, esterase;
ADH, alcohol dehydrogenase;
GST, glutathione-S-transferase;
SOD, superoxide dismutase;
GSP, glutathione peroxidase,
CAT, catalase;
LAC, laccase;
GHF, glycohydrolase family;
PL, pectin lyase;
CBM, carbohydrate binding module.

The lignase/detox transcripts were distributed across the two libraries (24 wood library, 25 lignin library, and 47 common to both libraries; Table 1). In contrast, glycosyl hydrolase expression profiles were skewed towards the wood library (94 wood library, 37 lignin library, and 35 common to both libraries; Table 1).

### Example 5

### Lignase & Detox Candidates

Differentially expressed lignase and detox transcripts, identified based on previous studies (Tartar et al. 2009; Coy. M. R. et al. Insect Biochem. Mol. Biol. 40, 723 (2010); Scharf, M. E. et al. PLoS One 6, e21709 (2011)) include cytochrome P450s (P450), esterases (EST), alcohol dehydrogenases (ADH), glutathione-S-transferases (GST), superoxide dismutases (SOD), glutathione peroxidases (GSP), catalases (CAT), and laccases (LAC) (Table 1).

The enrichment of transcripts encoding these enzyme families in the wood and depolymerized lignin-fed libraries strongly indicates that wood and lignin contain xenobiotic constituents that must be detoxified and/or metabolized in the termite gut (Tartar et al. 2009; Scharf & Boucias 2010). P450s are important oxidative xenobiotic-metabolizing enzymes; a total of 28 were enriched among the two subtracted libraries. P450 families represented in order of abundance are Cyp6 (n=13), Cyp4 (8), Cyp9 (2), Cyp12 (2), Cyp15 (2) and Cyp49 (1). These results expand the known number of P450s in R. flavipes to over 40 (Zhou, X. et al. Insect Mol. Biol. 15, 749 (2006); Tartar et al. 2009; Tarver, M. R. et al. BMC Mol. Biol. 11, 28 (2010)), which approaches the total number of known P450s in other eusocial insects such as honey bees (Honeybee Genome Sequencing Consortium 2006) and xylophagous leaf-cutter ants (Suen, G. et al. PLoS Genetics 7, e1002007 (2011)).

Fifteen differentially responsive esterase transcripts were also identified, which supports earlier work suggesting esterases, e.g. ferruloyl esterases, as a potentially relevant family (Wheeler et al. 2010). Similarly, 39 other antioxidant enzymes from the ADH (19), GST (10), SOD (3), GSP (7) and CAT (2) classes that play important roles in xenobiotic defense were also identified.

Two laccases were also identified, which is in agreement with previous recombinant protein studies showing significant roles by termite gut laccases in catalyzing lignin related phenol-oxidase activity and hemicellulose digestion (Coy et al. 2010; Scharf et al. 2011). One laccase transcript was enriched in each of the two subtracted libraries, the lignin-alkali-associated laccase (contig 05192). These laccase results provide supporting evidence to substantiate the selective feeding approach and use of differential transcript abundance data to identify novel lignase candidates (Scharf & Boucias 2010).

Seventeen aldo-keto reductases (AKRs) with differential expression in both libraries were also identified, as shown in Table 1. The AKRs are known to act on lignin and other phenolic by products in ligno-cellulolytic yeast (Kuhn et al. 1995; Ford et al. 2001). With 448 total reads, one AKR was the 18^{th} most highly expressed transcript identified (contig 00057), had 1.6-fold higher expression in the lignin library, and its protein product increased with lignin feeding, indicating that AKR plays important physiological roles in the termite gut, and more specifically, in lignin/phenolic metabolism. Additionally, 126 novel candidate lignase and associated cofactor transcripts were discovered based on fifty-six microbial lignase literature references and related sequence databases. These additional auxiliary enzymes include: 54 dehydrogenases, 18 oxidases, 4 peroxidases, 1 transhydrogenase, 13 reductases, 5 hydratases, 9 hydrolases, 2 dioxygenases, 4 hydroxylases, 2 thiolases, 5 synthetases and 13 redoxins.

### Example 6

### Transcripts Encoding Carbohydrate Active Enzymes

Three categories of carbohydrate active transcripts were differentially expressed among the wood and depolymerized lignin libraries, as shown in Table 1, specifically: glycosyl hydrolases (cellulases, hemicellulases, chitinases, ceramidase, amylases and laminarinases); carbohydrate binding proteins (carbohydrate binding. EF hand, and lectin); and pectin lyases. Many (57%) of differentially expressed carbohydrate active transcripts (94 of 166) were enriched in the wood library. Surprisingly, some (210%) of carbohydrate active transcripts (35 of 166) were enriched in the depolymerized-lignin library.

Hemicellulase-coding GH transcripts would likely be enriched when paper (cellulose)-associated transcripts were subtracted from the wood and lignin-fed libraries. Indeed, transcripts matching hemicellulases from glycosyl hydrolase families (GHF) 11, 3, 5, 26, 43 and 28 were most abundant in the wood library. However, protist symbiont cellulases from GHF 7 and 45 were also enriched in the wood library, in part supporting previous predictions that protist GH enzymes are required to degrade more recalcitrant cellulose forms (i.e., microcrystalline cellulose; Todaka et al. 2007, 2010) and/or play dual roles in cellulose and hemicellulose depolymerization (Zhou. X. et al. Gene 395, 29 (2007)).

Chitinases (GHF 8 and 18) were also enriched in the wood-fed library, possibly in association with termite defense against wood-associated fungi that contain chitin polymers as a component of their cell walls (Hamilton, C. et al. J. Insect Physiol. 57, 1259 (2011)).

In the depolymerized lignin library, enriched GHF transcripts included cellulases from GHF 7, 45, 1 and 9, and hemicellulases from GHF 11, 26, 43 and 28. Two R. flavipes cellulases from GHF 1 and 9 have been functionally characterized: the Cell-1 cellulase from GHF9 and ft-glu beta glucosidase from GHF1 (Zhou et al. 2007; Zhou, X. et al. Arch. Insect Biochem. Physiol. 74, 147 (2010); Scharf, M. E. et al. Insect Biochem. Mol. Biol. 40, 611 (2010)). These are both endogenous host enzymes with high expression in symbiont-free salivar) gland tissue; recombinant forms of both enzymes were found to act synergistically in saccharification of complex lignocellulose and hemicellulose substrates.

Interestingly, induction of GHF members by depolymerized lignin (a non-carbohydrate phenylpropanoid-derived material) suggests that termites and their gut symbiota compensate for phenol-associated lignocellulose recalcitrance by producing more cellulases. In this respect, the results support that a rich pool of symbiont cellulases is produced to maximize release of available sugars and provide metabolic energy to overcome the lignin barrier (Todaka, N. et al. FEMS Microbiol. Ecol. 59, 592 (2007); Todaka, N. et al. PLoS One 5, e8636 (2010)).

While not wishing to be bound by any one hypothesis, as initially suggested by functional studies with a recombinant laccase, cellulase and β-glucosidase (Scharf et al., 2011), and supported by the current transcriptomic findings, host cellulases appear to be part of a detoxification pathway in which free sugars released from cellulose/hemicellulose are conjugated to toxic mono-lignols released from lignin. The latter “conjugative detoxification” hypothesis would transform the view of host-symbiont collaboration in termite lignocellulose digestion (because it would indicate that host cellulases play broader roles in detoxification of lignin by-products, in addition to their stereotypical roles in cellulose digestion and nutrition.

### Example 7

### Proteomics

Proteomics was used to determine lignin feeding impacts at the post-translational level. For this purpose, depolymerized lignin and cellulose feeding assays occurred under identical conditions as described above for pyrosequencing. After bioassays, soluble gut protein fractions were subjected to 2D SDS-PAGE, followed by LC-MS/MS analysis. In this analysis, the focus was on soluble proteins and thus mitochondrial and microsomal protein fractions which have membranous detoxification enzymes such as P450s were excluded.

Several depolymerized lignin-inducible proteins were identifiable by comparison to (1) the pyrosequencing database resulting from the present study, and (2) an existing termite gut EST database (Tartar et al. 2009). Ten differentially expressed proteins were identified, including: aldo-keto reductase, profilin, ELF-1, G3P dehydrogenase, arginine kinase. Cell-1 endoglucanase (apparent multimers and degradation products), pyruvate phosphate dikinase, thaumatin, angiotensin converting enzyme, and cyclophilin.

Among the proteins identified by homology searches, the most differentially expressed protein was aldo-keto reductase (AKR) (contig 00057). This lignin-induced AKR was chosen for further study because AKRs can be oxidative detox enzymes involved in the metabolism of phenolic compounds like those found in lignin (Kuhn et al. 1995; Ford et al. 2001).

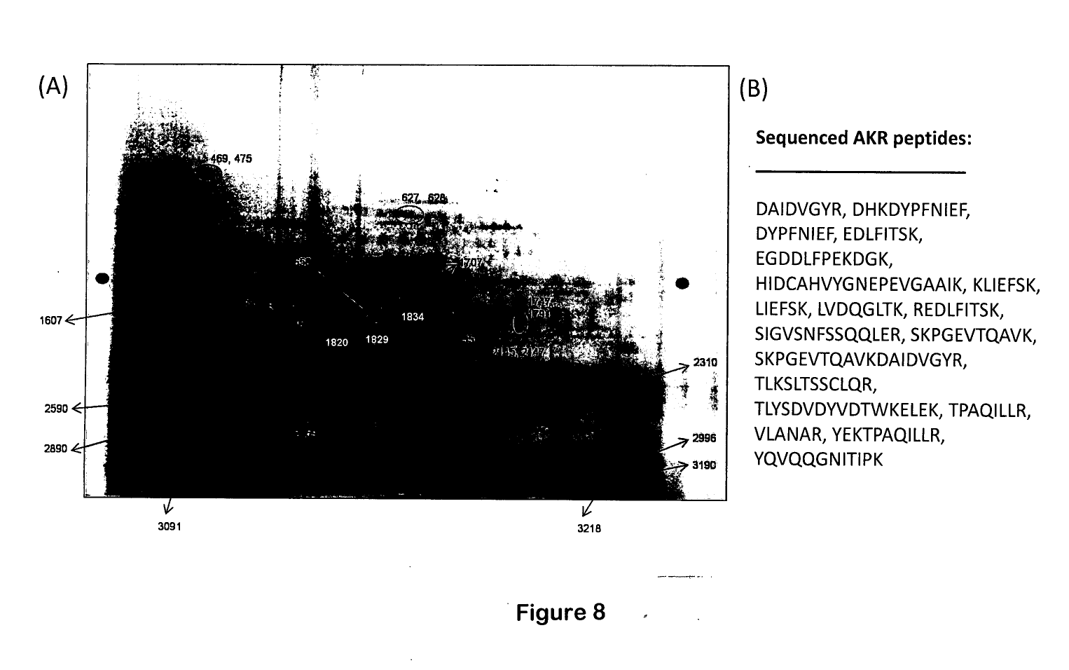

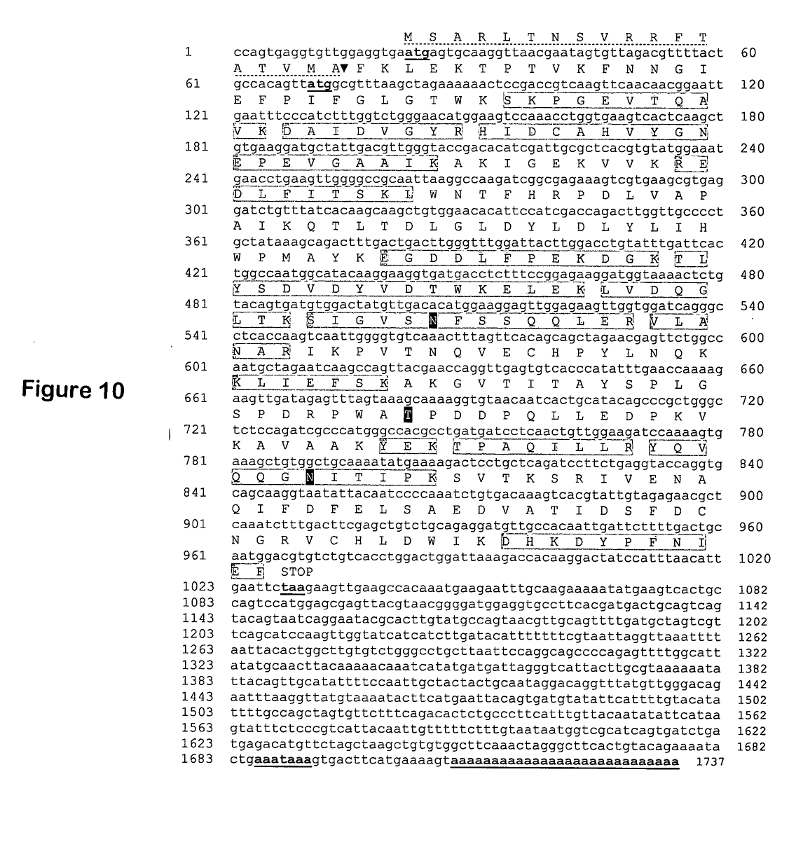

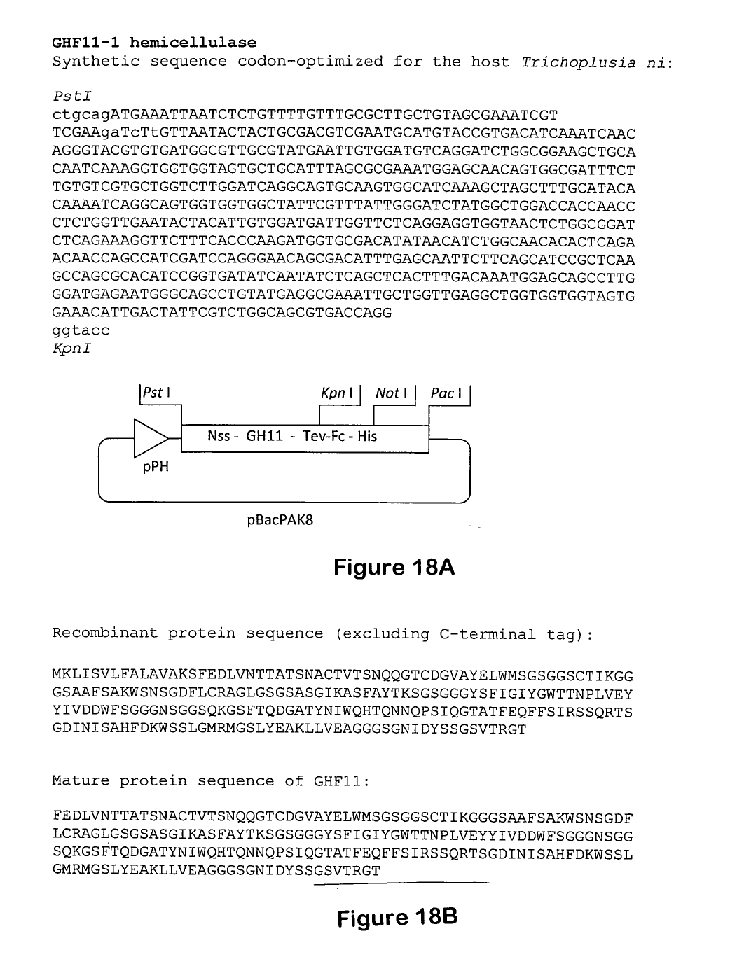

The nineteen sequenced AKR peptides (FIG. 8B) match to translated AKR cDNA sequences from the present pyrosequencing work (Table 1) and to a translated R. flavipes cDNA library we published previously (Tartar et al. 2009) (FIG. 18). The translated AKR cDNA is shown in FIG. 10 along with sequenced peptide alignments.

There are two predicted translational initiation sites in the AKR cDNA sequence. The first translational initiation site would produce a protein of 37.9 kDa with pI of 6.46, and the second site would produce a protein of 36.1 kDa with pI of 5.72. The estimated molecular weight and pI values for the AKR peptides in 2D SDS-PAGE gels ranged from 35.5-35.6 kDa and 5.45-5.79 respectively. Thus, it is likely that the second translational initiation site is used.

In silico signal peptide analysis predicted that this AKR does not have a secretion signal peptide, and protein targeting analysis indicates that this AKR is cytoplasmic. These results therefore do not suggest that AKR is secreted into the termite gut lumen to interact directly with lignin.

These proteomics findings are significant because they (1) emphasize AKRs as a potentially important family of expressed termite lignocellulases, and (2) show congruence at the transcription and translational levels. The latter result further validates the use of a selective feeding bioassay approach (Scharf & Boucias 2010) for identification of novel termite lignocellulases.

### Example 8

### Functional Studies with Recombinant Enzymes

Two host cellulases, Cell-1 and β-glu, whose transcripts were found to be inducible, were shown in previous recombinant enzyme studies to act synergistically in the saccharification of various lignocellulose substrates (Scharf et al. 2011). Also, another inducible transcript identified in the current study, the LacA laccase, was previously found to metabolize lignin phenolic compounds and enhance saccharification of hemicellulose when tested in combination with Cell-1 and β-glu (Coy et al. 2010; Scharf et al. 2011). These results, which initially established that the host transcripts Cell-1, β-glu and LacA play significant digestive roles in the termite gut, are strengthened by the current findings.

Using a modified experimental design that compared wood vs. cellulose feeding, a digestome microarray study has identified a number of the same wood-responsive enzyme-encoding transcripts as the current study; for example, AKR, P450, CAT, EST, GST, SOD, GPX, LAC, GHF7 and GHF11 (Table 1). Based on the combined microarray and current pyrosequencing results, four novel recombinant enzymes were generated and tested in combination with Cell-1[contig 00577], β-glu [contig 00343] and LacA [contig 05192].

These novel enzymes included a protozoan symbiont GHF7 cellulase (GHF7-3 [contig 00237]), a GHF11 endo-xylanase (GHF11-1 [contig 03644]), a catalase (CAT [contig 01463]), and the aldo-keto reductase noted above (AKR [contig 00057]). Histidine tagged recombinant enzymes were each engineered into recombinant baculoviruses and expressed in Trichoplusia ni larvae after oral infection (Liu, Y. et al. Chap. 13 in. Murhammer, D. (ed.) Baculovirus and insect cell expression protocols (Humana Press, Towata N.J., pp. 267-280, 2007); Kovaleva, E. S. et al. Biotechnol. Lett. 31, 38 (2009)).

Construct Generation, Recombinant Protein Production, and Purification:

Recombinant proteins were produced in whole Trichoplusia ni larvae using the PERLXpress procedure described previously (O'Connell et al., 2007; Kovaleva et al., 2009, each of which is herein incorporated by reference in its entirety). For the Lacasse A. C-terminal tags composed of two glycine and six histidine residues, as well as XbaI and EagI restriction sites, were incorporated into target gene amplicons utilizing the primers shown in Table 2.

TABLE 2

| Primer | | |
|---|---|---|
| No.: | Target | PCR Primer Sequence^{a} |
| 1 | Laccase A | 5′- |
| | (LacA)- | tctagaATGTTGCCTTGCGTCCTGCTTG-3′ |
| | forward | |
| 2 | Laccase A | 5′- |
| | (LacA)- | cggccgTTAGTGATGATGGTGATGATGacctcc |
| | reverse | GTTGGTGTTCACGGGAGGTGT-3′ |
| 3 | AKR- | 5′- |
| | forward | tctagaATGAGTGCAAGGTTAACGAATAGTG-3′ |
| 4 | AKR | 5′-tctagaATGGCGTTTAAGCTAGAAAAA-3′ |
| | truncated- | |
| | forward | |
| 5 | AKR- | 5′- |
| | reverse | cggccgTTAGTGATGATGGTGATGATGacctcc |
| | | GAATTCAATGTTAAATGGATAGTCCTTG-3′ |
| 6 | GHF7-3 | 5′- |
| | forward | GATCAGATCTTAATCAGGATTTCACCTACAC- |
| | | 3′ |
| 7 | GHF7-3 | 5′- |
| | reverse | GATCGGTACCATAAGTGCTATCAATCGGAC-3′ |

^{a}XbaI and Eagi sites are underlined in primers 1-5; BglII site underlined in primer 6; KpnI site underlined in primer 7; start and stop codons are indicated in bold; His-6 and Gly-2 encoding regions are italicized.

The nucleotide sequences encoding catalytically active GHF11. AKR, and CAT were generated by oligonucleotide synthesis in their entireties and inserted in the expression vector. The GHF7-3 gene was PCR-amplified from the a full-length clone TS51-B10 using primers (see Table 2) introducing Bgl II (forward) and Kpn I (reversed) sites for the cloning of the GHF7-3 gene lacking native signal sequence into a pre-made vector comprising viral signal sequence derived from gp64 envelope protein gene and further including a thrombin-cleavable C-terminal His-tag.

The PCR amplicons or synthetic polynucleotides, including encoded ORF cDNA sequences of target proteins, plus the C-terminal Gly-His tag (for Laccase A), were cloned into the XbaI-EagI sites of the pVL1393 transfer vector, and recombinant baculoviruses were generated using a homologous recombination system in insect Sf9 cells, pVL1393 was used only for cell-1 and laccase A insertion and expression: pBacPAK8 and 9 were used for the catalase, AKR, GHF7-3 and GHF11-1; pFastBac1 was used for βGlu expression

Plasmid TS51-B10 was used as a template for GHF7-3 amplification. The sequence was not complete, lacking the N-terminal signal sequence and no ATG. Using alignments to several sequences from GenBank it was deduced that 16 nucleotides were likely missing. The gene was expressed with the viral signal sequence, because the complete native signal sequence was unknown.

To confirm protein expression after viral infection. Sf9 cultures were screened by western blotting using an anti-His-specific monoclonal antibody (Novagen, Madison, Wis.). Active viral lines were identified as described previously (O'Connell et al., 2007; Kovaleva et al., 2009, each of which is herein incorporated by reference in its entirety) and subsequently injected into T. ni larvae for large-scale protein production.

Recombinant protein was recovered from clarified T. ni homogenates to near homogeneity, as described previously (Coy et al. 2010; Scharf et al. 2010; Zhou et al. 2010, each of which is herein incorporated by reference in its entirety) by tandem Ni-IMAC (nickel-immobilized metal affinity chromatography) followed by buffer exchange with Sephadex G-25 chromatography. Protein storage buffer consisted of 0.1 M sodium acetate, 0.15 M sodium chloride, 5 mM calcium chloride, and 5 μM copper sulfate (pH 5.8). Laccase purity was assessed by SDS-PAGE with Coomassie staining and western blotting with anti-His tag antibody. All protein concentrations were determined using a microplate Bradford assay (Bio-RAD; Hercules, Calif.).

Recombinant proteins were used directly in pine sawdust digestion assays with glucose detection following an established protocol (Scharf et al. 2011). Xylose detection was performed using a commercial D-Xylose assay (Megazyme; Wicklow, Ireland).

In agreement with previous results (Scharf et al. 2011); (1) the recombinant Cell-1 and β-glu combination liberated significant glucose release relative to negative controls that lacked enzyme, and (2) addition of the LacA laccase caused no significant increase in glucose release from pine lignocellulose, as shown in FIG. 2. For three-enzyme cocktails that included Cell-1 and β-glu plus AKR, CAT or GHF7-3, non-significant (about 1.5-3-fold) increases in glucose release occurred. However, four-enzyme cocktails that included either AKR or CAT plus all three cellulases (Cell-1, β-glu and GHF7-3) significantly increased glucose release by more than 3.5-fold relative to Cell-1 and fi-glu alone. Three and four-enzyme cocktails that included LacA had slightly reduced glucose output relative to identical reactions without LacA. As expected, GHF11-1 did not significantly enhance glucose release when tested alone and in combination against pine lignocellulose; however. GHF11-1 did catalyze significant xylose release from pine lignocellulose, as shown in FIG. 3. Additionally, after a 4-hr pre-incubation period with the LacA laccase. GHF11-1 liberated significantly greater xylose release presumably as a result of LacA-mediated lignin disassociation (FIG. 3). These results show that three non-cellulase enzymes identified through our selective feeding and quantitative pyrosequencing approach (AKR, CAT and LacA), significantly enhance lignocellulose saccharification by host and symbiont cellulases and hemicellulases, including in the termite gut.

### Example 9

### Bioassays and 1D-PAGE Analysis

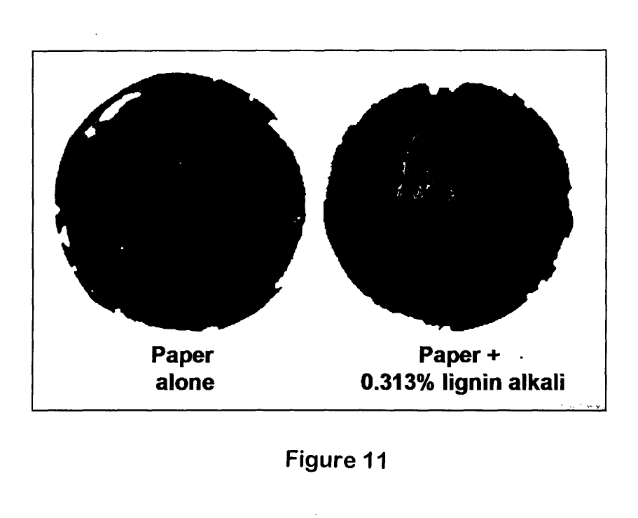

Bioassays were conducted with 50 worker termites (R. flavipes colony K9) on Whatman #1 filter papers in 50 mm diameter Petri plates. Two treatments were tested: 98% cellulose paper alone and paper+0.313% lignin alkali (Sigma-Aldrich #471003). This concentration was tested based on previous results showing significantly increased gut phenol-oxidase activity after feeding on filter paper+0.313% lignin alkali (Tartar et al., (2009)). The lignin alkali solution was prepared in water and adjusted to pH 7.4 with acetic acid. After 7 days considerable feeding occurred on both substrates, as shown in FIG. 11. A surface feeding pattern was seen in the lignin treatment, which is consistent with “gnawing pheromone” activity elicited by the phenolic compound hydroquinone seen in Reticulitermes termites (Reinhard et al., (2002), J. Chem. Ecol. 28: 1). Over 90% survival occurred in both treatments.

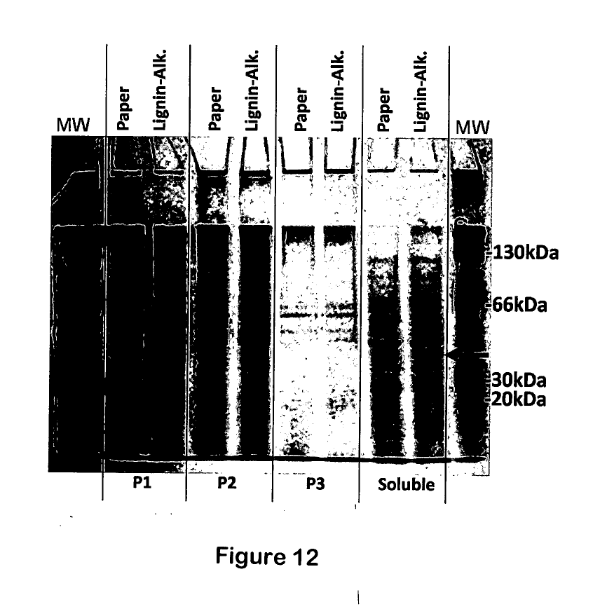

Next, whole guts were isolated from all surviving termites, placed in sodium acetate buffer (0.1 M, pH 7) and homogenized using a glass-glass Tenbroeck tissue grinder. Nuclear and mitochondrial fractions were pelleted by centrifugation at 1,000×g and 10.000×g, respectively, and the microsomal fraction pelleted at 10,000×g in homogenization buffer+8 mM calcium chloride (Kupfer & Levine (1972) Biophys. Biochem. Res. Comm. 47: 611). All protein fractions from both treatments were assessed for protein content by standard Bradford protein assays. Protein quality was assessed by one-dimensional SDS-PAGE, as shown in FIG. 12. Some differences in mitochondrial and soluble protein composition among treatments were observable, particularly in the range of about 35 kDa in the soluble supernatant fraction.

### Example 10

### 2D-PAGE Analysis

The soluble gut supernatant fractions (FIG. 12, right) were subjected to 2D PAGE analysis as follows:

(a) Protein Preparation for CyDye Labeling:

One ml of gut soluble protein mixture in 0.1M sodium acetate pH 7 with 8 mM calcium chloride, from either paper or lignin fed termites, was precipitated with 9 volumes of ice cold 10% TCA/acetone overnight at −20° C. The resulting protein pellet was recovered by centrifugation at 20,000 g for 20 min at 4° C. and was washed twice with 80% ethanol then was washed twice with 80% acetone. The protein pellet was air dried on ice for 5 mins and was dissolved in DIGE labeling buffer (8M urea 2M thiourea, 4% CHAPS, 20 mM Tris pH 8.5, 0.2% SDS). Benzonase (Novagen) was added to each dissolved extract to digest large molecules of nucleic acid. The resulting solution was then clarified at 40,000 g for 30 min at 15° C. before protein quantification assay. Protein concentration was determined using EZQ® protein quantification kit (Invitrogen) and ovalbumin as standard.

(b) CyDye Labeling:

Protein labeling with CyDye was modified and performed according to Friedman et al., (2004) Proteomics 4: 793-811, using commercially available CyDye technology (GE Healthcare). After adjusting sample solution to pH 8.5, each protein sample was covalently linked to a different CyDye fluorophore, such as Cy2 to reference sample mixture (a mixture of equal amount of protein extracts from both paper and lignin), Cy3 to paper, and Cy5 to lignin. In each case 100 μg of protein was labeled with 400 pmol CyDye for 30 min in darkness on ice. Excess dye was quenched with 1 μl of 10 mM lysine.

(c) 2-D Gel Electrophoresis:

All three different CyDye labeled samples were mixed together with 200 μg of unlabeled mixed sample and increased to 500 μl with IEF buffer (8 M urea, 2M thiourea, 4% CHAPS, 100 mM DDT, 0.2% SDS, 0.5% IPG, buffer pH 3 to 11) before passively rehydrating a 24 cm IPG no-linear gradient strip (pH 3 to 11; GE Healthcare). Labeled proteins in the strip were focused at 19° C. on an IPGphor3 Unit (GE Healthcare) with voltage ramping up to and held at 10,000 Volt for a total 100 kVh. After IEF, the strip was first equilibrated with reducing buffer (50 mM Tris-HCl pH 6.8, 6 M Urea, 30% glycerol, 2% SDS, 100 mM DTT), then equilibrated with alkylation buffer (50 mM Tris-HCl pH 6.8, 6 M Urea, 30% glycerol, 2% SDS, 2.5% iodoacetamide). Both equilibration steps were held at room temperature in darkness for 15 mins. After equilibration, the strip was transferred and mounted on top a 24×24 cm, 8 to 16% Tris Glycine polyacrylamide gel (Jule) under a layer of warm 0.5% agarose made in SDS electrophoresis running buffer. Electrophoresis was carried out in Ettan Daltsix Unit (GE Healthcare) at 12° C. at 10 mA/gel for one hour, and then overnight at a constant current of 12 mA/gel and a limit of 150 V until the dye front reached the bottom of the plate.

Results:

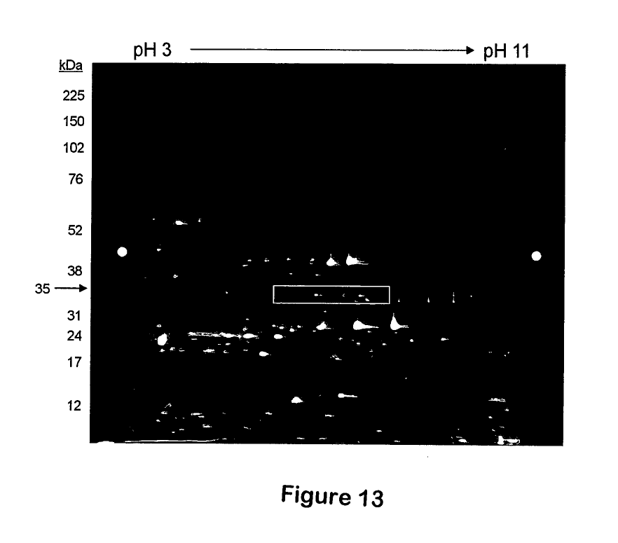

Because the paper and lignin alkali samples were labeled, respectively, with Cy3 (green) and Cy5 (red) dyes, this enabled two-color quantification on single gels. With this approach, green labeled protein spots are up-regulated with paper diet, red labeled spots are up-regulated with lignin diet, and yellow spots are identical between diets. 2D PAGE revealed several candidate lignin-inducible proteins; however, the most prominently up-regulated protein spots had molecular masses near 35 kDa and pI values in the 2-5 range (see box in FIG. 13).

### Example 11

### Peptide Expression Analysis and Spot Picking

Methods of peptide expression analysis and spot picking are as follows:

Image Acquisition and Data Analysis:

Immediately after gel electrophoresis, CyDye labeled proteins in gels were scanned using a Typhoon 9400 Variable Mode Imager (GE Healthcare). The excitation/emission wavelengths for Cy2, Cy3 and Cy5 were 488/520, 532/580 and 633/670 nm respectively. Three images (internal standard, paper fed, and lignin fed) were acquired. The digital image information acquired was then analyzed with DeCyder 2D software, version 7.0, (GE Healthcare). All spots present in all images in the gel were co-detected, matched, and normalized with the DIA (Differential In-Gel Analysis) Module within the software. There were over 2000 spots detected and matched. Interesting spots were selected by setting the fold difference threshold to 1.5 fold. Specifically, any protein spot from the lignin sample that was expressed above or below 1.5 fold when compared with the identical spot from the paper sample was selected. A pick list was made and the ordinance information obtained from DeCyder software for each interesting protein spot was transferred to an automated ProPic spot picker (Genomic Solutions) using the pick list. The spots then were excised by the picker and transferred to a collecting plate and were used for protein identification as described in the following section.

Results:

Over 2000 protein spots were identifiable on 2D gels. Twenty-three spots with greater or less than 2-fold induction or repression in the lignin treatment were selected for robotic spot picking, as shown in FIG. 8A, and subsequent MS/MS analysis. Three spots with similar expression were also selected as controls (#1834, 627 and 628).

### Example 12

### Protein Identification

Methods for protein identification were as follows:

(a) Protein Identification by LC-MS/MS:

Protein identification was performed by LC-MS/MS. Trypsin-digested samples were injected onto a capillary trap (LC Packings; PepMap Inc.) and desalted for 5 min with a flow rate of 3 μl/min of 0.1% v/v acetic acid. The samples were loaded onto an LC PACKING® C18 Pep Map nanoflow HPLC column. The elution gradient of the HPLC column started at 3% solvent A, 97% solvent B and finished at 60% solvent A, 40% solvent B for 30 mins. Solvent A consisted of 0.1% v/v acetic acid, 3% v/v ACN, and 96.9% v/v H₂O; Solvent B consisted of 0.1% v/v acetic acid, 96.9% v/v ACN, and 3% v/v H₂O.

LC-MS/MS analysis was carried out on a LTQ ORBITRAP XL® mass spectrometer (Thermo Scientific). The instrument, under control of Xcalibur 2.07 with LTQ Orbitrap Tune Plus 2.55 software, was operated in the data dependent mode to automatically switch between MS and MS/MS acquisition. Survey scan MS spectra (from m/z 300-2000) were acquired in the orbitrap with resolution R=60,000 at m/z 400. The five most intense ions were sequentially isolated and fragmented in the linear ion trap by collision-induced dissociation (CID) at a target value of 5.000 or maximum ion time of 150 ms. Dynamic exclusion was set to 60 sees. Typical mass spectrometric conditions include a spray voltage of 2.2 kV, no sheath and auxiliary gas flow, a heated capillary temperature of 200° C. a capillary voltage of 44V, a tube lens voltage of 165V, an ion isolation width of 1.0 m/z, a normalized CID collision energy of 35% for MS2 in LTQ. The ion selection threshold was 500 counts for MS2. An activation q=0.25 and activation time of 30 ms were set.

(b) Protein Search Algorithm:

All MS/MS spectra were analyzed using Mascot (Matrix Science. London, UK; version 2.2.2). Mascot was set up to search R. flavipes gut and symbiont EST databases (Genbank Accession Nos. FL634956-FL640828 and FL641015-FL645753) and termite gut 454 contig data sets (present study), assuming the digestion enzyme trypsin. Mascot was searched with a fragment ion mass tolerance of 0.50 Da and a parent ion tolerance of 15 ppm. The iodoacetamide derivative of Cys, deamidation of Asn and Gin, and oxidation of Met are specified in Mascot as variable modifications. Scaffold (version Scaffold-02-03-01, Proteome Software Inc.) was used to validate MS/MS based peptide and protein identifications. Peptide identifications were accepted if they could be established at greater than 95.0% probability as specified by the Peptide Prophet algorithm. Protein identifications were accepted if they could be established at greater than 99.0% probability and contained at least 2 identified unique peptides. Protein probabilities were assigned by the Protein Prophet algorithm.

Results:

In total, 26 protein spots were selected for analysis, as shown in Table 3. Four spots could not be identified because of limited quantities (#1650, 2890, 1641 and 1607). Several differentially expressed proteins were identifiable by comparison to existing termite gut EST database (Tartar et al. (2009)) and pyrosequencing database (present study). The differentially expressed proteins identifications included: (1) aldo-keto reductase, (2) profilin, (3) ELF-1, (4) G3P dehydrogenase, (5) arginine kinase, (6) Cell-1 endoglucanase (apparent multimers and degradation products), (7) pyruvate phosphate dikinase, (8) thaumatin, (9) angiotensin converting enzyme, and (10) cyclophilin.

TABLE 3
Protein identities as determined by tandem MS analysis of trypsin digested protein spots.
Identifications were by comparison of peptide fragments to translated termite gut and
symbiont EST and 454 pyrosequencing databases. Aldo-keto reductases highlighted in italics

| | | | | Lignin/paper | | | |
|---|---|---|---|---|---|---|---|
| spot | | Spot | | (Cy5/Cy3) spot | Max | | Mass |
| # | Identity | No. | Abundance | Volume Ratio | Volume | pI | (kDa) |
| 1 | ?? unknown | 1650 | Increased | 5.71 | 473559 | 5.54 | 37482 |
| 2 | aldo-keto reductase | 1820 | Increased | 5.12 | 2525836 | 5.45 | 35599 |
| | (contig 572) | | | | | | |
| 3 | Profiling | 3190 | Increased | 4.44 | 3827247 | 6.59 | 7157 |
| 4 | ELF-1 | 2310 | Increased | 4.41 | 763914 | 8.75 | 22078 |
| 5 | Profiling | 3218 | Increased | 3.98 | 3917813 | 8.11 | 6874 |
| 6 | aldo-keto reductase | 1829 | Increased | 3.67 | 11104035 | 5.62 | 35491 |
| | (contig 572) | | | | | | |
| 7 | G3P dehydrogenase | 1747 | Increased | 3.42 | 1357365 | 6.73 | 36508 |
| 8 | G3P dehydrogenase | 1837 | Increased | 3.41 | 3336722 | 5.76 | 35408 |
| 9 | G3P dehydrogenase | 1791 | Increased | 3.23 | 1206698 | 6.73 | 35946 |
| 10 | ?? unknown | 2890 | Increased | 3.07 | 1923337 | 4.02 | 11748 |
| 11 | ?? unknown | 1641 | Increased | 2.68 | 525509 | 5.45 | 37614 |
| 12 | Arginine kinase | 1707 | Increased | 2.62 | 415012 | 5.52 | 36814 |
| 13 | Cell-1 | 3091 | Increased | 2.56 | 3581093 | 4.64 | 8447 |
| 14 | Pyruvate phosphate | 1746 | Increased | 2.46 | 439945 | 5.41 | 36319 |
| | dikinase 2 | | | | | | |
| 15 | thaumatin | 2590 | Increased | 2.38 | 2682508 | 3.91 | 17512 |
| 16 | Cell-1 | 1835 | Increased | 2.28 | 2800085 | 6.21 | 35436 |
| 17 | aldo-keto reductase | 1834 | Similar | 2.24 | 3575686 | 5.79 | 35463 |
| | (contig 572) | | | | | | |
| 18 | Hex-2 | 627 | Similar | 1.73 | 851046 | 5.9 | 69062 |
| 19 | Hex-2 | 628 | Similar | 1.67 | 661848 | 5.9 | 69201 |
| 20 | ?? unknown | 1607 | Decreased | −2.32 | 2394470 | 3.88 | 36111 |
| 21 | Angiotensin | 475 | Decreased | −2.38 | 413431 | 4.9 | 93824 |
| | converting enzyme | | | | | | |
| 22 | Cell-1 | 2147 | Decreased | −2.43 | 20546481 | 6.43 | 27850 |
| 23 | Cell-1 | 2259 | Decreased | −2.46 | 13142836 | 5.39 | 23318 |
| 24 | Cyclophilin | 2996 | Decreased | −2.66 | 2582515 | 8.64 | 9925 |
| 25 | Cell-1 | 469 | Decreased | −2.68 | 307876 | 4.85 | 93824 |
| 26 | Cell-1 | 2145 | Decreased | −2.78 | 17425842 | 6.43 | 27850 |

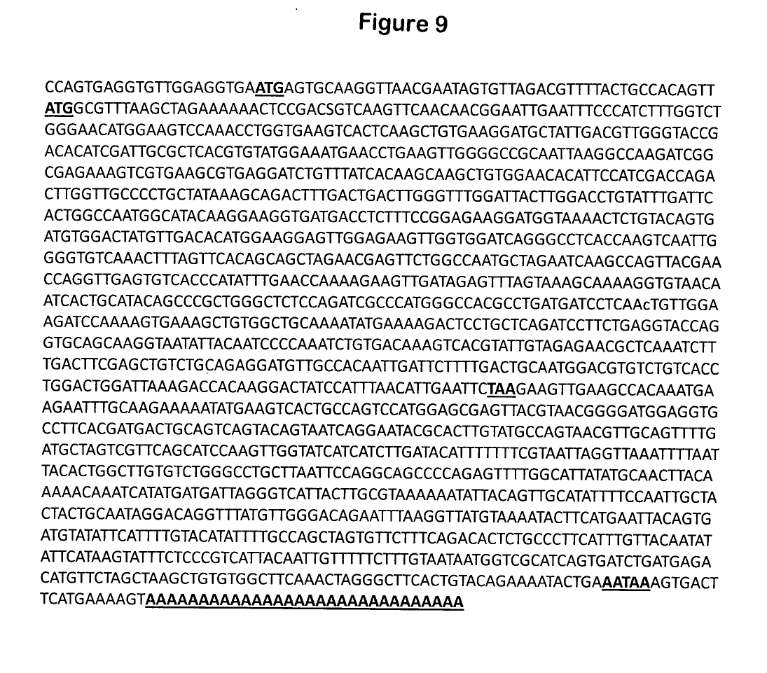

While other proteins as noted in Table 3 may ultimately prove to be relevant to lignocellulose digestion, the efforts here focused on the aldo-keto reductases (AKRs). Specifically, two AKR peptides (spots 1820 and 1829) had the highest lignin alkali induction of around about 5-fold, and a third AKR peptide was up-regulated 2.24 fold by lignin alkali. The nineteen sequenced AKR peptides, as shown in FIG. 8B, are a near-full-length match for a full-length cDNA sequenced previously from a R. flavipes host gut cDNA library (FIG. 9) (contig 572; Tartar et al., (2009)). The translated AKR cDNA is shown in FIG. 10, along with sequenced peptides. The predicted mass for the full-length amino acid sequence is 37.8 kDa with a pI=6.2; whereas, the predicted mass and pI of the sequence encoded from the second start codon are 36 kDa and 5.7, respectively. The mass and pI values for the sequenced protein spots ranged from 35.5-35.6 kDa with pI values of 5.45-5.79); thus, it is likely that the native protein sequence begins at the second methionine start codon.

The same AKR transcript was also obtained through quantitative pyrosequencing efforts (contig 00057) from both of the cellulose-subtracted, wood and lignin-alkali libraries. However, while present in both libraries, the AKR sequence was encountered 1.6-fold more frequently in the lignin alkali library (270 lignin alkali library, 178 wood library). Also, with 448 total reads, the AKR transcript was the eighteenth most highly sampled transcript in the pyrosequencing study. Such high gut expression levels indicate the AKR protein to be physiologically important. Indeed, AKRs are enzymes with established links in the literature to metabolism of phenolic compounds such as those that occur in lignin.

### Example 13

β-glu cellulase gene RfBGluc was PCR-amplified form the clone (GenBank FL635576; ADK12988.1) using the following primers: forward, 5′-GTCGACATGAGGTTACAGACGGGTTGC-3′ (Sal1 sites underlined, start codon shown in bold); reverse, 5′-CTGCAGTTAGTGATGATGGTGATGAGGTCTAGGAAGCGTTCTGGAA-3′ (Pst1 site underlined, stop codon shown in bold and 6× histidine-coding nucleotides italicized). The PCR amplicon encoded the full-length RfBGluc ORF sequence (amino acids 1 to 495) and 6× histidine tag at the C-terminus and was cloned into Sal1-Pst1 sites of Bac-to-Bac transfer vector pFastBac 1. Baculovirus was prepared using Bac-to-Bac system in Sf9 cells according to manufacturer protocol and injected into Trichoplusia ni larvae.

### Example 14

GHF 1 contig sequence was based on overlapping est-sequences FL642851.1, FL644625.1, FL644617.1, FL641536.1 of the Reticulitermes flavipes symbiont library termite gut metagenome cDNA. Baculovirus was prepared using homologous recombination system in Sf9 cells according to manufacturer protocols and injected into Trichoplusia ni larvae.

### Example 15

GHF9 Cell-1 was PCR-amplified from R. flavipes cDNA with forward primer 5′-CTAGTCTAGACTAGATGAAGATACTCCTGCTATfGCATTAAVTGTGTCAACAGTAATGTGGGTGT CAACAGCTGCTGAGACTATAAG-3′ (Xba1 site underlined, start codon in bold, heterologous signal sequence italicized), reverse

5′-
TTTCCTTTTGCGGCCGCTTAGTGATGATGGTGATGATGCACGCCAGCCTT
GAGGAG-3′

(NotI site underlined, stop codon in bold, 6×His italicized). The PCR amplicon encoded the ORF for the Cell-1 with the exchanged signal sequence for Bombyx mori (silk moth) hormone bombyxin A-6 (GENE ID: 100169714 Bbx-a6) signal sequence and the C-terminal 6×His tag, and was cloned into XbaI-NotI sites of the pVL1393 transfer vector.

Baculovirus was prepared using homologous recombination system in Sf9 cells according to manufacturer protocol and injected into Trichoplusia ni larvae.

### Example 16

Laccase 6 and 12 genes were PCR-amplified form clones (GenBank GQ421909 and GQ421911) using forward primer 5′-tctagaATGTTGCCTTGCGTCCTGCTTG-3′ (XbaI sites underlined, start codon in bold) and reverse 5′-cggccgTTAGTGATGATGGTGATGATGacctcc-GTTGGTGTTCACGGGAGGTGT-3′ (EagI sites underlined, His-6 and Gly-2 italicized and stop codon in bold). The PCR amplicons encoded full-length RfLac1 and RfLac2 plus the C-terminal His-tag, and were cloned into XbaI-EagI sites of the pVL1393 transfer vector.

Baculoviruses were prepared using homologous recombination system in Sf9 cells according to manufacturer protocol and injected into Trichoplusia ni larvae.

### Example 16

### Superoxide Dismutase and Glutathione Peroxidase Sequences

The genes for both superoxide dismutase and glutathione peroxidase sequences were significantly upregulated by wood and lignin feeding relative to paper (cellulose) feeding in two independent studies, both of which are herein incorporated by reference in their entireties (Sethi A. et. al., (2013) Insect Biochemistry and Molecular Biology, 43: 91-101; Raychoudhury R. et. al., (2013) Insect Molecular Biology, 22: 155-171) R. flavipes Superoxide Dismutase was originally termed “SOD-2”, and developed from TG_Contig 272 (Tartar et al. 2009) based on 3 ESTs (Genbank accession numbers): FL636626 FL636951 FL636064. BlastX identities of original TG_Contig 272 are shown in Table 4.

TABLE 4
Sequences producing significant alignments:

| | | Max | Total | Query | E | Max | |
|---|---|---|---|---|---|---|---|
| Accession | Description | score | score | coverage | value | ident | Links |
| XP 002116945.1 | expressed hypothetical protein | 121 | 191 | 58% | 3e−40 | 71% | [symbol] |
| | [Trichoplax adhaerens] | | | | | | |
| | >gb\|EDV20519.1\| expressed | | | | | | |
| | hypothetical protein [Trichoplax | | | | | | |
| | adhaerens] | | | | | | |
| AAV85459.1 | extracellular Cu/Zn superoxide | 126 | 187 | 62% | 3e−39 | 60% | |
| | dismutase [Lasius niger] | | | | | | |
| ZP 926169.1 | PREDICTED: hypothetical protein | 117 | 187 | 58% | 4e−39 | 71% | [symbol] |
| | isoform 4 [Mus musculus] | | | | | | |
| | >ref\|XP_994787.1\| PREDICTED: | | | | | | |
| | hypothetical protein [Mus mysculus] | | | | | | |
| | >gb\|EDK98801.1\| mCG1036425 [Mus | | | | | | |
| | musculus] | | | | | | |
| NP 001156153.1 | hypothetical protein LOC100162795 | 116 | 186 | 60% | 5e−39 | 68% | [symbol] |
| | [Acyrthosiphon pisum] | | | | | | |
| | >dbj\|BAH71286.1\| ACYPI003921 | | | | | | |
| | [Acyrthosiphon pisum] | | | | | | |
| AAW29025.1 | copper/zinc superoxide dismutase | 116 | 186 | 58% | 7e−39 | 68% | |
| | [Epinephelus coioides] | | | | | | |
| ACR56338.1 | Cu/Zn-superoxide dismutase | 119 | 186 | 58% | 9e−39 | 66% | |
| | [Hemibarbus mylodon] | | | | | | |
| | >gb\|ACR56339.1\| Cu/Zn-superoxide | | | | | | |
| | dismutase [Hemibarbus mylodon] | | | | | | |

Final SOD2-his ORF (636 bp).

Thrombin-His tag is double underlined. Start and Stop codons are bold and underlined.

ATGAAGGAAACACGGGTTCTGATGATATTTCTGCTCGTGGCTATGACTGC
TGCCCAGTATCCAGTACGTTATTATGTACAACAGCCAGGTGACCCTGAAC
ACCACGTCAGCTGCCCTAAATCAGCTGTGTGTAACTTGATTCCTTCTAAG
GACTCTACAGTCTCTGGACAAGTACAGCTGTACCAGGCATCTCAGGCTGA
ACCAGTTGAAATAATAGTTTCGGTACAAGGTCTGAAACCACCAGGGCTAC
ACGGTTTTCATCTTCATCGTGATGGCAATACTGATGATGACTGCAAAGCA
GCTGGGCCACATTTCAATCCATTCAATCACACACATGGTGGGCCAGAAGA
TGAATTCCGACATGCAGGAGACTTTGGAAACATATTGGCAGATGAATATG
GTAATGCTTCTTACCGTATAAAGGGAACACAGATCTCaCTATGTCCTGGC
AGTGTGGGTTACTCTGTGGGCCGTGCTTTTGTGGTGCATGAAGGCATAGA
TGATcTTGGTAAAGGTGGTAATGAAGAGTCTCTCAGAACCGGGAATGCTG
GAGGTCGCCTTGCTTGCTGTGTTGTTGAAGCTGTCAATTACGGTACCCTT
GTGCCCAGAGGCAGCCATCATCACCACCATCACTAA

SOD2-his Recombinant Protein (211 AA, 22.8 kDa, pI 6.5, Charge −3.6).

The signal peptide is underlined, the recombinant Thrombin-Histidine tag is indicated in italics, and the Copper/Zinc superoxide dismutase signature 2 motif is double underlined. The signal peptide was predicted by the SignalP-4.0 eukaryotic prediction server and the Cu/Zn SOD signature motif predicted using Prosite (http://prosite.exnasy.org/).

MKETRVLMIFLLVAMTAAQYPVRYYVQQPGDPEHHVSCPKSAVCNLIPSK
DSTVSGQVQLYQASQAEPVEIIVSVQGLKPPGLHGFHLHRDGNTDDDCKA
AGPHFNPFNHTHGGPEDEFRHAGDFGNILADEYGNASYRIKGTQISLCPG
SVGYSVGRAFVVHEGIDDLGKGGNEESLRTGNAGGRLACCVVEAVNYGTL
VPRGSHHHHHH.

R. flavipes glutathione peroxidase was originally termed “GPX-1” and developed from contig00784 [length=1185, numreads=51](Sethi et al. 2013. Insect Biochem Mol Biol 22: 155-171) based on 51 individual 454 sequence reads. The top BlastX identities of GPX-1/contig00784 are shown in Table 5.

TABLE 5

| | Max | Total | Query | E | | |
|---|---|---|---|---|---|---|
| Description | score | score | cover | value | Ident | Accession |
| hypothetical protein TcasGA2 TC010362 [Tribolium | 279 | 279 | 49% | 3e−89 | 68% | EFA01372.1 |
| castaneum] | | | | | | |
| glutathione peroxidase [Danaus plexippusi] | 276 | 276 | 49% | 4e−88 | 68% | ERJ74560.1 |
| phospholipid hydroperoxide glutathione | 263 | 263 | 42% | 2e−83 | 74% | XP 002429001.1 |
| peroxidase, putative [Pediculus humanus corporis] | | | | | | |
| >gb\|EEB16253.1\| phospholipid hydroperoxide | | | | | | |
| gluthathione peroxidase, putative \|Pediculus | | | | | | |
| humanus corporis\| | | | | | | |
| gluthathione peroxidase \|Bombyx mori\| | 263 | 263 | 47% | 4e−83 | 65% | NP 001036999.1 |
| >dbj\|BAE07196.1\| gluthathione peroxidase \|Bombyx | | | | | | |
| mori] | | | | | | |
| PREDICTED: probable phospholipid hydroperoxide | 260 | 260 | 49% | 8e−82 | 65% | XP 003427378.1 |
| gluthathione peroxidase-like [Nasonia vitripennis] | | | | | | |
| gluthathione peroxidase [Papilio xuthus] | 258 | 258 | 42% | 1e−81 | 72% | BAM18713.1 |
| gluthathione peroxidase [Helicoverpa armigera] | 258 | 258 | 42% | 2e−81 | 72% | AFC98365.1 |

Final GPx1-HIS ORF (630 base pairs). Thrombin-His tag is double underlined Start and Stop codons are bold and underlined. No GenBank accession number is yet available.

ATGCTGGTTGCAGGACTGCAGTTATTTGGAACTGCATTTTGCGCTCTGCG
ACTCTTGTCCACACGTACCGCCCcAGTCATGGCGGCAACTCCAGAAGATT
GGAAAAaTGCATCATCCATCTATGATTTCACTGTAAAAGATATAAAGGGG
CAAGATGTGTCTCTGGAGAAATACAGAGGCGACGTGGCCATTATTGTGAA
TGTAGCTTCAAAGTGTGGCCTGACTCCCACAAATTACAAGGAATTGGCAG
AGCTTCATGACAAGTATGCTGAATCGAAAGGTCTCCGCATATTAGCTTTC
CCTTGCAACCAGTTTAATAGCCAGGAACCTGGAAACGCGGAGGAAATAGT
GTGCTTTGCCAAGTCTAAGAATGCAAATTTTGACATGTTTGAGAAGATTG
ATGTCAATGGAAACCATGCACATCCACTGTGGAAGTACTTGAAGCACAAG
CAAGGAGGAACTCTAGGAGATTTCATCAAGTGGAATTTTACCAAGTTCAT
CATTGACAAGAATGGACAGCCTGTAGAGAGGCATGGTCCCAACGTTGATC
CCAGTAAACTGGTGTCCAGCTTGGAGAAGTACTGGGGTACCCTTGTGCCC
AGAGGCAGCCATCATCACCACCATCACTAA

GPx1-his Recombinant Protein (630 AA, 51.7 kDa, pI 4.8. Charge −4.3)

The signal peptide is underlined, the recombinant Thrombin-Histidine tag is italicized. The signal peptide was predicted by the SignalP-4.0 eukaryotic prediction server.

MLVAGLQLFGTAFCALRLLSTRTAPVMAATPEDWKNASSIYDFTVKDIKG
QDVSLEKYRGDVAIIVNVASKCGLTPTNYKELAELHDKYAESKGLRILAF
PCNQFNSQEPGNAEEIVCFAKSKNANFDMFEKIDVNGNHAHPLWKYLKHK
QGGTLGDFIKWNFTKFIIDKNGQPVERHGPNVDPSKLVSSLEKYWGTLVP
RGSHHHHHH.

1. A system for producing a fermentable product from a lignified plant material, the system comprising either: (i) the catalytically active domains, or polypeptides comprising said catalytically active domains, of cellulases Cell-1 and β-glu, and further comprising a catalytically active domain of at least one enzyme, wherein said enzyme is a cellulase, an aldo-keto-reductase, a catalase, or a laccase; or (ii) the catalytically active domains, or polypeptides comprising said catalytically active domains of a endo-xylanase, a superoxide dismutase, and a glutathione peroxidase and, optionally, a laccase, and wherein the catalytically active domains, or polypeptides comprising said catalytically active domains, cooperate to provide a fermentable product from a lignified plant material.

2. The system of claim 1, wherein at least one of Cell-1, β-glu, a cellulase, a aldo-keto-reductase, a catalase, a laccase, a endo-xylanase, a superoxide dismutase, and a glutathione peroxidase is derived from a termite.

3. The system of claim 1, wherein at least one of Cell-1, β-glu, a cellulase, a aldo-keto-reductase, a catalase, a laccase, a endo-xylanase, a superoxide dismutase, and a glutathione peroxidase is expressed from a recombinant nucleotide sequence.

4. The system of claim 3 comprising a series of isolated recombinant polypeptides, wherein each polypeptide comprises a catalytically active domain and is expressed from an expression vector of a recombinant expression system, and wherein the recombinant expression system is selected from a eukaryotic cell-based system.

5. The system of claim 4 wherein the eukaryotic cell-based system is a baculovirus system.

6. The system of claim 3 comprising a series of isolated recombinant polypeptides, wherein each polypeptide comprises a catalytically active domain and is expressed from an expression vector of a recombinant expression system, and wherein the recombinant expression system is selected from a prokaryotic cell-based system.

7. The system of claim 1, wherein the system comprises the catalytically active domains, or polypeptides comprising said catalytically active domains, of: (i) Cell-1, β-glu, cellulase GHF7-3, LacA, aldo-keto-reductase, and a catalase; (ii) Cell-1, β-glu, cellulase GHF7-3, and a catalase; (iii) Cell-1, β-glu, cellulase GHF7-3, and aldo-keto-reductase; (iv) Cell-1, β-glu, cellulase GHF7-3, and LacA; (v) Cell-1, β-glu, and cellulase GHF7-3; (vi) Cell-1, β-glu, and a catalase; (vii) Cell-1, β-glu, and aldo-keto-reductase; or (viii) LacA and GHF11-1, and wherein the system can optionally comprise a superoxide dismutase and/or a glutathione peroxidase.

8. (canceled)
